(19) United States
(12) Patent Application Publication
(10) Pub. No.: US 2023/0175032 A1
(43) Pub. Date: Jun. 8, 2023

(54) EFFICIENT SYNTHESIS OF OMEGA-GLYCOSIDES AND ALKYL GLYCOSIDES

(71) Applicants: AMPHISTAR, Lievegem (BE); UNIVERSITEIT GENT, Gent (BE)

(72) Inventors: Wim SOETAERT, Lovendegem (BE); Sophie ROELANTS, Melle (BE); Christian STEVENS, Schelderode (BE); Elisabeth DELBEKE, Mariakerke (BE); Goedele LUYTEN, Waasmunster (BE); Melike PALA, Gent (BE); Jelle REMMERY, Wichelen (BE)

(21) Appl. No.: 17/921,963

(22) PCT Filed: May 12, 2021
(86) PCT No.: PCT/EP2021/062756

§ 371 Date: Oct. 27, 2022

(30) Foreign Application Priority Data
May 13, 2020 (EP) 20174471.1

(51) Int. Cl. C12P 19/44; C12N 9/04; C12N 9/10; C12P 7/6409

(52) U.S. Cl. CPC C12P 19/44; C12N 9/0006; C12N 9/1048; C12P 7/6409; C12Y 101/0302; C12Y 114/14; C12P 7/18

(57) ABSTRACT

The present invention relates to the field of production of novel biosurfactants. More specifically, the present invention relates to the efficient generation of short chained on-glycosides with less than 10%, preferably less than 1%, ω-1 glycosides using a fungal strain such as the yeast Starmerella bombicola having a dysfunctional CYP52M1 cytochrome P450 monooxygenase and a dysfunctional FAO1 fatty alcohol oxidase to produce high amounts of so-called unsaturated (symmetrical) α,ω-bola glycosides free from contaminating α,ω-1 bola glycosides, and subjecting said unsaturated (symmetrical) α,ω-bola glycosides to conditions inducing the breaking of the present double bond(s) such as for example through ozonolysis performed in water. More specifically, the present invention discloses the generation of (acetylated) C9:0 ω-sophoroside aldehydes, C9:0 ω-glucoside aldehydes, C9:0 ω-glucolipids, C9:0 ω-sophorolipids, C9:0 ω-sophoroside alcohols and C9:0 ω-glucoside alcohols and their further derivatives. The present invention also discloses methods to produce alkyl sophorosides in increased ratios.

### TECHNICAL FIELD OF INVENTION

The present invention relates to methods to efficiently produce useful omega-glycosides such as—for example—sophorosides and glucosides, which are useful as (bio)surfactants and/or possess antimicrobial properties. More specifically, the present invention discloses methods involving a conversion step to produce unsaturated alpha,omega-bola glycosides with less than 10% contaminating (unsaturated) alpha,omega-1-bola glycosides followed by a step in which said unsaturated alpha,omega-bola glycosides are subjected to reaction conditions to break at least one cleavable bond. The latter method results in high yield production of omega-glycosides with less than 10% contaminating omega-1-glycosides.

### BACKGROUND OF THE INVENTION

Dwindling supply of fossil resources and the need to address the environmental concerns associated with their use require the development of sustainable, safe and eco-friendly technologies for the production of chemicals (Sheldon, 2014; Tuck et al., 2012). Consumer pressure for green and sustainable alternatives is becoming an increasingly important driver of the market. Utilizing renewable biomass as the feedstock while adhering to the other principles of green chemistry (Anastas & Eghbali, 2010) is considered to be the option to achieve these goals. However, the production of bio-based chemicals should also have an economically competitive edge compared to that of fossil-derived chemicals to facilitate the transition from the current fossil-based economy to a circular bio-based economy (Philp et al., 2013). The commercial development of bio-based chemicals would be more likely by producing new-to-industry chemicals with superior properties (as opposed to conventional analogues or drop-ins) and/or by improving the efficiency of their production (e.g. increasing productivity of fermentation processes and/or reducing the number of reaction steps leading to the desired end-product of chemical (derivatization) processes) (Farmer & Mascal, 2015). Either of these goals could be realized by harnessing the structure and the functionality of the bio-based feedstock in the end-product. Biosurfactants or bio-based surfactants are an example of such bio-based biochemicals. Biosurfactants can be produced through chemical or biological means and bacterial or microbial biosurfactants have been identified as one of the top emerging biobased products to be commercialized before 2030 (Fabbri et al., 2018). Biosurfactants constitute a diverse group of molecular structures and types, but have in common that they all have at least one hydrophilic and at least one hydrophobic part, which gives them amphiphilic properties. The field of commercialized microbial biosurfactants is quite limited in types and producers/suppliers. Summarized, the commercialized products are glycolipids (sophorolipids (SLs), rhamnolipids (RLs) and mannosyl erythritol lipids (MELs)), lipopeptides, phospholipids/fatty acids and particulate and polymeric biosurfactants.

Glycosides (GSs) are a class of compounds where one or more carbohydrate molecules is/are covalently bound to at least one other compound via a glycosidic bond. The ‘other compound’ can be—for example—a (functionalized) aliphatic chain of carbon molecules. Glycosides can be produced through chemical means e.g. through traditional Fischer synthesis from glucose and fatty alcohols yielding alkyl poly glucosides (McCurry et al., 1996) or through biological means e.g. through (whole cell) biocatalytical conversions from for instance glucose and fatty alcohols yielding bola—and/or alkyl/alcohol—sophorosides (SSs) and—glucosides (GLuSs) (Van Renterghem et al., 2018). A combination of chemical and biological methods e.g. the further biocatalytical glycosylation of chemically synthetized alkyl poly glucosides (Adlercreutz et al., 2012). Alkyl sophorosides (alkyl SSs), bola sophorosides (bola SSs), bola glucosides (bola GLuSs), alkyl glucosides (alkyl GLuSs) are non-limiting examples of glycoside microbial biosurfactants.

Glycolipids (GLys) are a class of compounds where one or more carbohydrate molecule(s), is/are covalently bound to a lipid molecule. Glycolipids can be produced through chemical means e.g. starting from sucrose and fatty acids (Yamagishi et al., 1974) to sucrose esters or through biological means e.g. (whole cell) biocatalytical conversion from for instance glucose and fatty acids/plant oils to SLs (Roelants et al., 2019; Siebenhaller et al., 2018).

Sophorolipids (SLs), bola sophorolipids (bola SLs), glucolipids (GLs), rhamnolipids (RLs), mannosylerythritol lipids (MELs) are non-limiting examples of glycolipid microbial biosurfactants.

The abovementioned glycoside- and glycolipid microbial biosurfactants can be produced using (modified) microbial strains using renewable carbon sources e.g. first-generation biomass such as glucose, plant oil, fatty acids/-alcohols (Lodens et al., 2020; Roelants et al., 2019; Saerens, Zhang, et al., 2011; Van Renterghem et al., 2018), and even from waste- and side streams such as waste cooking oil, molasses, food waste, etc. (Kaur et al., 2019; Roelants et al., 2019; M. Takahashi et al., 2011).

Sophorolipids (SLs) are one of the best known microbial biosurfactants and one of the first ones to be commercialized. S. bombicola is the preferred applied yeast strain towards SL production as it produces SLs with high titers (above 200 g L⁻¹) (Roelants et al., 2019; Van Renterghem et al., 2018), while productivities of over 2 g/L·h have been reported (Dolman et al., 2017; Gao et al., 2013). The specific surface-active properties of SLs have directed their use to relevant application areas (D. W. G. Develter & Lauryssen, 2010; Lourith & Kanlayavattanakul, 2009) and are currently commercially applied in several ecological cleaning solutions and personal care products. SLs were also reported to possess bioactive properties (e.g. antimicrobial, antiviral, antifungal, . . . ) (Kim et al., 2002; Roelants et al., 2019; Sen et al., 2017) and have for that specific reason also found application in particular cosmetic/personal care products such as anti-acne soap and deodorants. Other microbial glycolipid biosurfactants, such as e.g. rhamnolipids have also recently been commercialized. The overall market volumes and commercial adaptation of microbial biosurfactants remains limited though, which is mainly because of two reasons: their higher costs and the lack of molecular variety. The cost aspect is mainly caused by low production scales and non-optimized and inefficient production technologies. The commercial adoption of (microbial) biosurfactants could thus be accelerated by increasing efficiencies and by increasing the variety of available compounds/molecular structures. Molecular variety translates into a variety of physicochemical/biological properties, which will subsequently translate into a higher number of potential application areas (Roelants et al., 2014). Increasing the molecular variety can be realized through genetic engineering the microbes aiming to produce new-to-nature biosurfactants and/or by chemical and/or enzymatic modification of (existing) microbial biosurfactants. A combination of both strategies can also be applied.

With regards to the chemical modification of specific types of microbial biosurfactant glycolipids/glycosides and more specifically of SLs, the groups of Gross et al. (SyntheZyme), Delbeke et al. (Ghent University) and Develter et al. (Ecover) described methods for the use of wild type (lactonic) SLs produced by the yeast Starmerella bombicola as starting materials for subsequent chemical derivatization (Delbeke, Movsisyan, et al., 2015; D. Develter & Fleurackers, 2011; Gross et al., 2013; Van Bogaert et al., 2011). SLs are naturally a mix of omega-(ω) and omega-1 (ω-1) SLs, with the main compound produced by S. bombicola being ω-1 C18:1 SLs of which the lactonic version is shown in FIG. 1A (D. Develter & Fleurackers, 2008; Konishi et al., 2008; Tulloch et al., 1962). The abovementioned groups described the use of SLs as precursors aiming to increase variety and generate a portfolio of new biosurfactant compounds using chemical- and/or chemoenzymatic routes. This resulted in a large library of new compounds e.g short chain (omega-1) sophoroside aldehyde, short chain (omega-1) sophorolipids, omega-1 quaternary ammonium sophorolipids (QASLs), SS amine oxides, SS amines, bolamphiphile SSs, etc. with interesting physicochemical and biological (e.g. antimicrobial) properties (Delbeke, 2016; Delbeke et al., 2018; Delbeke, Lozach, et al., 2016; Delbeke, Movsisyan, et al., 2015; Delbeke, Roelants, et al., 2016; Delbeke, Roman, et al., 2015; D. Develter & Fleurackers, 2008; Gross et al., 2013; Van Bogaert et al., 2011). Besides exhibiting antimicrobial properties, a number of the synthesized QASLs showed high transfection efficacies as well (Delbeke, Lozach, et al., 2016; Delbeke, Roman, et al., 2015). Obviously, such applications are expected to create higher value/allow higher costs as compared to application in household detergents. The described chemical derivation routes starting from wild type SLs (mixture of ω-1 and ω, mainly ω-1), typically follow quite elaborate synthetic derivatization pathways. For example in the method described by (Delbeke, Roman, et al., 2015) the yeast derived lactonic SLs are first converted into SL methyl esters and are peracetylated (to protect the sugar head group) before being subjected to ozonolysis to break the double bond present in the SLs, to in the end obtain the ω-1 C9 sophoroside aldehyde building block (with minor amounts of ω C9 sophoroside aldehyde present) and a C9 alkyl chain split off as side product, which has to be removed (Delbeke, Roman, et al., 2015). This last aspect negatively impacts the yields for this derivatization method starting from wild type SLs, as 30% (9 out of 30) of the initial carbon atoms are lost in this process. Moreover, the C9 side product must—as mentioned—be removed from the C9 sophoroside aldehyde product, which adds both additional steps (again reducing yields) and costs to the process. Despite these hurdles, the abovementioned ω-1 C9 sophoroside aldehyde building blocks were further derivatized into a portfolio of biosurfactants as already mentioned above. These derivatives were thus also a mixture of ω-1 (mainly) and ω-glycosides. So far, the production of a diverse range of ω-glycosides based on a bioprocess step combined with chemical and/or enzymatic derivatizations resulted in a mixture wherein mainly ω-1 glycosides are present and wherein ω-glycosides are present in very low amounts. The reason for the low abundancy of ω-glycosides present in the mixture is due to the fact that the enzymatic hydroxylation of fatty substrates by most wild type SL producing microorganisms, such as S. bombicola, is quite specific, since the relative amounts of hydroxylation at the w or ω-1 position is correlated with the length of the built in aliphatic molecule. A double bond for example spatially shortens the fatty acid chain length, causing oleic acid to be both ω and ω-1 (mainly) hydroxylated, whereas stearic acid apparently is too long for ω-hydroxylation and is therefore exclusively hydroxylated at the ω-1 position (D. Develter & Fleurackers, 2008; Konishi et al., 2008; Tulloch et al., 1962). This enzymatic hydroxylation occurs through the activity of the CYP52M1 enzyme in the case of S. bombicola (Van Bogaert et al., 2013) and its functional homologue(s) in other SL producing microbial species.

The genetic engineering of SL-producing yeast strains can offer some solutions to some of these issues.

For instance, Van Renterghem et al. (2018) recently showed that the production of bolaform sophorosides (mix ω and ω-1) can be achieved by the S. bombicola knockout strain ΔatΔsbleΔfao1 (Van Renterghem et al., 2018). These compounds were also reported to be produced by another S. bombicola knock out strain i.e. Δfao1 by Takahashi et al. (2016). The bolaform SSs or bola SSs comprise two identical hydrophilic sophorose head groups both linked glycosidically to a hydrophobic (unsaturated) hydrocarbon linker. The double bond present on the unsaturated hydrocarbon linker is—likewise as is the case for wild type SLs—susceptible to cleavage, e.g. by ozone, which would theoretically result in the production of two shorter chained sophoroside units (mix ω and ω-1) with aldehyde (sophoroside aldehydes), alcohol (sophoroside alcohols) or carboxylic acid (sophorolipids) functionality at the alpha position depending on the process conditions for cleavage of the double bond. This would circumvent the abovementioned loss of the alkyl part split of in the case of using wild type SLs. Although these compounds produced by these new strains could thus offer some solutions, the resulting shorter chained sophoroside units and derivatives thereof also constitute of a mixture of w and ω-1 compounds of which the ratio (w/ω-1) is prone to variation in the upstream fermentation process and thus prone to batch to batch variation. This variation in ratios of compounds gives rise to varying properties of the specific mixture as even small molecular differences can have a dramatic impact on the corresponding properties of the derived products (Baccile et al., 2016; Roelants et al., 2019). Moreover, both abovementioned S. bombicola strains still produce several types of other (bola) SLs as side products (F. Takahashi et al., 2016; Van Renterghem et al., 2018), which thus again complicates derivatization and requires additional purification steps and again results in a loss of carbon during the process and thus low yields and high costs.

An example of a method to cleave double bonds is—as mentioned above—ozonolysis. Indeed, ozonolysis is an efficient method for the oxidative cleavage of double bonds in olefinic compounds (Frische et al., 2003; Goebel et al., 1957; Oehlschlaeger & Rodenberg, 1968). As such, ozonolysis is extensively applied on oleochemicals derived from vegetable oils (e.g. sunflower oil, rapeseed oil). Concerning production of bio-based chemicals via ozonolysis of oleochemicals, the intention has been to produce aldehydes, alcohols, mono- or dicarboxylic acids or their mixtures either as end-products or as functional intermediates (Hannen et al., 2013; Köckritz & Martin, 2011; Omonov et al., 2014; Tran et al., 2005). Ozonolysis also enables—as mentioned above—the formation of a functional short chain sophoroside aldehyde, short chain SL carboxylic acid (Delbeke, Roman, et al., 2015; D. Develter & Fleurackers, 2008) and many further derivatization options (Delbeke, Roman, et al., 2015). Despite the high atom efficiency of ozonolysis reactions and the decreased risks due to on-site generation of the oxidant, ozonolysis still suffers from safety concerns (Nobis & Roberge, 2011). The latter arises mostly from the generation of highly unstable by-products such as ozonides and peroxides which could be explosive (Nobis & Roberge, 2011). These issues can be tackled by careful consideration of the reactor system and the reaction conditions (Irfan et al., 2011). Moreover, the use of participating solvents, such as water or primary alcohols, can limit their formation. However, some oleochemicals suffer from low water solubility, thus limiting the use of the latter solution. This is also the case for wild type SLs. Although a derivatization method of wild type SLs based on ozonolysis in water has been disclosed, a number of steps and care are required to guaranty solubilization of lactonic SLs in a trade of with foaming issues (D. Develter & Fleurackers, 2011).

Another example of a method to cleave double bonds is through the action of enzymes. Indeed, enzymes are versatile proteins with a very broad range of activities. Enzymes cleaving double bonds have been described e.g. heme and non-heme oxygenases (Mutti, 2012). An example of a biocatalytical method applied industrially for the oxidative cleavage of aliphatic olefinic double bonds, is the use of two enzymes i.e. lipoxygenase and a fatty acid hydroxyperoxide lyase (Stolterfoht et al., 2019). Liopoxygenase catalyzes the peroxidation of unsaturated fatty acids and the resulting hydroxyperoxy fatty acids are further converted by hydroxylperoxide lyase into aldehydes and oxo-acids. The aldehydes can be further reduced to primary alcohols by reverse action of alcohol dehydrogenases or oxidized to the respective carboxylic acids using aldehyde oxidases. Both types of oxidoreductases are commercially available. Classic industrial methods apply plant extracts as sources of these enzymes. However, recently heterologous co-expression of such types of enzymes in microbial cell factories, such as S. cerevisiae, has emerged as a viable option to (co-) express these enzymes. Another biocatalytic method to cleave double bonds is epoxidation, which can be accomplished through oxidation using peroxidases and/or monoxygenases (Toda et al., 2014). The resulting epoxides can subsequently be converted into vicinal diols by epoxide hydrolases (Kotik et al., 2012). These diols can be further oxidized to ketone-alcohols or diketones by alcohol dehydrogenases or oxidatively cleaved by monoxygenases to aldehydes or carboxylic acids.

Sophoroside aldehydes are extremely versatile compounds towards further chemical—but also enzymatic derivatization. They can be further reduced to primary alcohols as mentioned above through the reversed action of alcohol dehydrogenases, oxidized to the respective carboxylic acids by applying aldehyde oxidases or aldehyde dehydrogenases. Further derivatization to a myriad of compounds—as mentioned above for chemical derivatization—can also be accomplished through biocatalytical means. All the described biocatalytic reaction(s) could also be integrated within the production process through (heterologous) expression of the respective enzymes in de sophorolipid/sophoroside production host.

To summarize in previous work employing ozonolysis to produce new bio-based molecules from SLs, a mixture of ω and ω-1 (mainly ω-1) sophoroside aldehydes, sophorolipid (di)carboxylic acids and/or sophoroside alcohols together with a split off alkyl part were typically obtained depending on the position of the olefinic double bond and the workup conditions (D. Develter & Fleurackers, 2008; Omonov et al., 2014). Such mixtures require elaborate downstream processing (e.g. separation, purification) thereby increasing the costs of the final products (ω-1 glycosides and ω-1 glycolipids and their derivatives). There is thus an urgent need to design a method which enables highly efficient, high yield (circumventing the loss of carbon), selective production of glycoside(s) and glycolipid(s) (building blocks) avoiding solubility issues and the produced mixtures of ω and ω-1 glycosides.

### SUMMARY OF THE INVENTION

The present invention solves one or more of the above described problems of the prior art. In an aspect, a method is provided to produce ω-glycosides which contain less than 10%, preferably less than 1%, ω-1 glycosides, ω-2 glycosides and/or ω-3 glycosides, wherein said ω-glycosides comprise a carbohydrate that is bound to a primary or terminal carbon atom of an aliphatic chain of carbons via a glycosidic bond, comprising the steps of:
a. conversion of (a) suitable substrate(s) with a suitable microbial strain to produce a broth comprising unsaturated α,ω-bola glycosides which contain less than 10%, preferably less than 1%, of α,ω-1 bola glycosides, α,ω-2-bola glycosides and/or α,ω-3-bola glycosides, wherein said microbial strain is a fungal strain that has been mutated to have a dysfunctional cytochrome P450 monooxygenase CYP52M1 or homologue thereof and a dysfunctional fatty alcohol oxidase FAO1 or a homologue thereof or wherein said microbial strain is a fungal strain that has been mutated to have a dysfunctional cytochrome P450 monooxygenase CYP52M1, a dysfunctional fatty alcohol oxidase FAO1 or a homologue thereof and a dysfunctional glucosyltransferase, that is responsible for the second glucosylation step in the sophorolipid biosynthetic pathway UGTB1 or a homologue thereof,
and wherein said unsaturated α,ω-bola glycosides comprise two carbohydrates that are each bound to a primary or terminal carbon atom of an aliphatic chain of carbons via a glycosidic bond, wherein said aliphatic chain of carbons comprises at least one double bond,
b. optionally purifying said unsaturated α,ω-bola glycosides from the broth of step a), and c. subjecting said unsaturated α,ω-bola glycosides within said broth according to step a) to an ozonolysis reaction or an enzymatic reaction that breaks at least one unsaturated cleavable aliphatic bond within said unsaturated α,ω-bola glycosides, or, subjecting said unsaturated α,ω-bola glycosides which are purified according to step b) to an ozonolysis reaction or an enzymatic reaction that breaks at least one unsaturated cleavable aliphatic bond within said unsaturated α,ω-bola glycosides.

The method offers some clear advantages. 1) the ‘bola’ nature of the α,ω unsaturated bola glycosides obtained in step a), gives rise to high yields (100% in case only one double bond is present in the unsaturated α,ω-bola glycosides) as the cleavage of the double bond gives rise to two shorter chained ω-glycosides instead of one (see FIG. 12A) avoiding the loss of carbon. 2) embodiments wherein the unsaturated α,ω-bola glycosides are ‘symmetrical’, meaning one double bond is present in the in the middle of the aliphatic linker (see FIG. 7), will give rise to high uniformities, as two identical ω-glycosides are obtained after cleavage of the double bond (see FIG. 7). 3) the hydrophilic nature of bola glycosides i.e. their high water solubility, significantly facilitates the development of green methods to cleave the double bond(s) as this can be performed in water without any adaptations or control to increase solubility etc. while also avoiding the use of organic solvents. Moreover, in case ozonolysis is used as a method to cleave the double bond, the execution of this method in water dramatically increases not only the green nature, but also the safety of the process.

A further aspect relates to a method for the production of alkyl glycosides, said method comprising the conversion of (a) suitable substrate(s) with a suitable microbial strain to produce a broth comprising alkyl glycosides, wherein said microbial strain is a fungal strain that has been mutated to have a dysfunctional cytochrome P450 monooxygenase, in particular CYP52M1 or a homologue thereof, and a dysfunctional fatty alcohol oxidase, in particular FAO1 or a homologue thereof, or wherein said microbial strain is a fungal strain that has been mutated to have a dysfunctional cytochrome P450 monooxygenase, in particular CYP52M1 or a homologue thereof, a dysfunctional fatty alcohol oxidase, in particular FAO1 or a homologue thereof, and a dysfunctional glucosyltransferase that is responsible for the second glucosylation step in the sophorolipid biosynthetic pathway, in particular UGTB1 or a homologue thereof, wherein said fungal strain has additionally been mutated to have one or more dysfunctional oxidizing enzymes responsible for ω-oxidation of long chain fatty alcohols and/or alkanes/alkenes, preferably one or more oxidizing enzymes selected from the group consisting of: A1 comprising the amino acid sequence set forth in SEQ ID NO:101 or a homologue thereof, A2 comprising the amino acid sequence set forth in SEQ ID NO:103 or a homologue thereof, A3 comprising the amino acid sequence set forth in SEQ ID NO:105 or a homologue thereof, A4 comprising the amino acid sequence set forth in SEQ ID NO:107 or a homologue thereof, A5 comprising the amino acid sequence set forth in SEQ ID NO:109 or a homologue thereof, A6 comprising the amino acid sequence set forth in SEQ ID NO:111 or a homologue thereof and A7 comprising the amino acid sequence set forth in SEQ ID NO:113 or a homologue thereof, wherein said fungal strain is preferably a natural sophorolipid producing fungal strain, more preferably a yeast selected from the group consisting of Starmerella (Candida) bombicola, Starmerella (Candida) apicola, Candida magnoliae, Candida gropengiesseri, Starmerella (Candida) batistae, Starmerella (Candida) floricola, Candida riodocensis, Candida tropicalis, Starmerella (Candida) stellata, Starmerella (Candida) kuoi, Candida sp. NRRL Y-27208, Pseudohyphozyma (Rhodotorula, Candida) bogoriensis sp., Wickerhamiella domericqiae and a sophorolipid-producing strain (of the Starmerella clade) (Santos et al., 2018).

The additional mutation in one or more of the oxidizing enzymes A1-A7, in particular in at least A3 and A4, more particularly in at least A3, A4 and A1, offers the advantage of a relative increase in the production of alkyl sophorosides (i.e. without or, with less or minimal, co-production of bola sophorosides).

Yet a further aspect relates to use of an enzyme A1 comprising the amino acid sequence set forth in SEQ ID NO:101 or a homologue thereof, an enzyme A3 comprising the amino acid sequence set forth in SEQ ID NO:105 or a homologue thereof or an enzyme or an enzyme A4 comprising the amino acid sequence set forth in SEQ ID NO:107 or a homologue thereof for the production of diols, preferably α,ω-diols.

The above and further aspects and preferred embodiments of the invention are described in the following sections and in the appended claims. The subject-matter of appended claims is hereby specifically incorporated in this specification.

### BRIEF DESCRIPTION OF FIGURES

FIG. 1: Examples of ω-1 glycolipids (A and C) (grey circles indicating the carboxylic functions present) and ω-1 glycosides (B and D). (A) wild-type lactonic sophorolipids (B) α,ω-1-bola sophorosides (C) bola sophorolipids (D) ω-1 sophoroside aldehyde.

FIG. 2: C9:0 ω-sophoroside aldehyde

FIG. 3: Important parameters for characterization of the Δcyp52M1Δfao1 strain (●), compared to the parental Δcyp52M1 strain (◯) in shake flask. The average values and respective standard deviations are presented of A) log(CFU/mL)s, B) pH and C) glucose concentration (g/L). 1.8 (w/v) % of oleyl alcohol was added after 48 h of cultivation as hydrophobic substrate.

FIG. 4: UPLC-ELSD chromatograms of end samples of shake flask experiments for the A) Δcyp52M1Δfao1 strain, the B) Δcyp52M1 strain and the C) Δcyp52M1Δfao1ΔugtB1 strain, all fed with 1.8 (w/v) % oleyl alcohol (all 3×diluted). Respective masses determined with UPLC-MS are depicted above the respective peaks. In bold, masses corresponding to alkyl SS are depicted.

FIG. 5: Proposed production pathway of alkyl sophorosides (SSs) in the Δcyp52M1Δfao1 strain when fed with oleyl alcohol. (1) Fatty alcohol oxidase fao1, (2) fatty aldehyde dehydrogenase FAD, (3) cytochrome P450 monooxygenase CYP52M1, (4) glucosyltransferase UGTA1, (5) glucosyltransferase UGTB1, (6) acetyltransferase AT and (7) SL transporter MDR. By knocking out the fool and cyp52M1 gene (indicated by crosses), the fatty alcohol is not converted towards the corresponding fatty aldehyde, nor to the corresponding diol (in this case 1,18-octadecenediol) nor are de novo fatty acids hydroxylated (indicated by cross) to then enter the SL biosynthesis. In this way, the accumulating alcohol is directly glucosylated by glucosyltransferases UGTA1 and UGTB1, which sequentially add glucose molecules on the fatty alcohol and alkyl glucoside backbone respectively. A non-acetylated alkyl SS is obtained, which should be acetylated by the acetyltransferase AT, giving rise to a mixture of non-, mono- and di-acetylated alkyl SSs. Finally, these glycosides are likely to be transported out of the cell, presumably by the MDR transporter responsible for secretion of wild type SLs (Van Bogaert et al., 2013).

FIG. 6: Overview of the production pathway using the Δcyp52M1Δfao1 strain and feeding it with oleyl alcohol. By knocking out the cyp52M1 gene, it was expected that only glycosylation could be performed as suggested in route A, giving rise to an acetylated mixture of alkyl SS (up to two acetylations), acetylations are represented as R groups. However, due to an unexpected enzymatic activity in S. bombicola, hydroxylation of the fatty alcohol was observed, which gave rise to acetylated bola SSs (up to four acetylations) after glycosylation. In the end, a mixture of mainly bola SSs and minor alkyl SSs was found. Hydroxylation occurred exclusively terminally (omega), in contrast to the ΔatΔsbleΔfao1 strain reported by Van Renterghem et al., (2018).

FIG. 7: Structural confirmed composition of the purified oleyl based (C18:1) bola SS produced by the Δcyp52M1Δfao1 strain. Only terminal (w) hydroxylated compounds are found to be produced.

FIG. 8: A) log(CFU/mL) and B) glucose concentration of the Δcyp52M1Δfao1 strain in function of cultivation time when different primary alcohols were fed (lauryl, myristyl, palmityl, stearyl or oleyl alcohol). The average values and respective standard deviations are presented. The respective 1.8 (w/v) % of alcohol was added after 48 h of cultivation.

FIG. 9: End UPLC-ELSD chromatograms for the Δcyp52M1Δfao1 strain, fed with 1.8 (w/v) % (A) lauryl alcohol, (B) myristyl alcohol, (C) palmityl alcohol, (D) stearyl alcohol or (E) oleyl alcohol after 48 h of cultivation. All the end broths are 3×diluted. Retention times are indicated for the alkyl (up to two possible acetylations) and bola SSs (up to four possible acetylations). In bold, the glycoside compounds are depicted when the respective fed alcohol was incorporated in bola SS (dark grey) and alkyl SS (black), alcohol GLuSs and alkyl GLuSs (light grey). The peaks with retention time <1 min correspond to a mixture of strong hydrophilic compounds present in the samples as sugars, proteins and salts.

FIG. 10: Bola SS (A) and alkyl SS production (B) of the Δcyp52M1Δfao1 strain in function of cultivation time, when fed with 1.8 (w/v) % of the respective primary alcohol after 48 h of cultivation. Production is expressed as the sum of peak areas (V·sec) determined by UPLC-ELSD. (C) Relative peak areas (%) of the end broths of the S. bombicola Δcyp52M1Δfao1 strain determined with UPLC-ELSD of the different glycoside types, fed with 1.8 (w/v) % of lauryl alcohol (black), myristyl alcohol (dark grey), palmityl alcohol (medium grey), stearyl alcohol (light grey) or oleyl alcohol (white) after 48 h of cultivation.

FIG. 11: The triple knock out strain Δcyp52M1Δfao1ΔugtB1 produces (acetylated) alpha,omega-bola glucosides (bola GLuSs or bola GS). The figure shows the pathway to synthesize (acetylated) symmetrical bola glucosides. The bottom figure shows the actual (acetylated) symmetrical bola glucosides C18:1. R═COCH3 or H.

FIG. 12: A) Schematic representation of the cleavage of the double bond of one molecule of symmetrical C18:1 α,ω-bola sophorosides using ozonolysis, resulting in the generation of two C9:0 ω-sophoroside aldehyde molecules. B) Schematic representation of examples of chemical derivatization routes of C9:0 ω-sophorosides. Depending on the ozonolysis process conditions C9:0 ω-sophoroside aldehydes, C9:0 ω-sophoroside alcohols or C9:0 w sophorolipids are generated, each of which can be converted in further derivatives by appropriate chemical-, enzymatic- and/or chemo-enzymatic processes of which a few non limiting examples are shown.

FIG. 13: NMR analysis after performing and ozonolysis reaction on, on top (1) C18:1 sophorolipid lactone giving rise to C9:0 ω-1-aldehydes and on below (2) C18:1 (non-acetylated) symmetrical α,ω-bola sophoroside giving rise to C9:0 ω-sophoroside aldehydes without the detection of C9:0 ω-1-sophoroside aldehydes. The peak at 21.3 ppm in the top NMR spectrum corresponds to the CH₃ group at the subterminal position (21.3 ppm, CH₃CH). There is not any peak at this chemical shift in the spectrum below. Thus only C9:0 ω-sophoroside aldehydes are formed after ozonolysis of the C18:1 α,ω-bola sophorosides described in this invention.

C9:0 ω-1-sophoroside aldehyde (top) ((S)-8-([2″,3′,3″,4′,4″,6′,6″-heptaacetoxy-2′-O-β-D-glucopyranosyl-β-D-glucopyranosyl]-oxy)-nonanal) ¹³C-NMR (100 MHz, CDCl₃): δ_{C} 20.3 (CH₃C═O), 20.4 (2xCH₃C═O), 20.5 (CH₃C═O), 20.6 (CH₃C═O), 20.6 (CH₃C═O), 20.7 (CH₃C═O), 21.3 (CH₃CH), 22.1 (CH₂CH₂(C═O)H), 24.6 (CH₂(CH₂)₂), 29.3 (CH₂(CH₂)₂), 29.4 (CH₂(CH₂)₂), 36.4 (CH₂CHCH₃), 43.7 (CH₂(C═O)H), 61.9 (CH₂OAc), 62.1 (CH₂OAc), 68.2 (CHOC), 68.7 (CHOC), 71.2 (CHOC), 71.5 (CHOC), 71.6 (CHOC), 72.9 (CHOC), 74.6 (CHOC), 77.3 (CHOC), 77.9 (CHOC), 100.3 (CH(O)₂), 101.2 (CH(O)₂), 169.3 (CH₃C═O), 169.4 (CH₃C═O), 169.6 (CH₃C═O), 169.9 (CH₃C═O), 170.1 (CH₃C═O), 170.4 (CH₃C═O), 170.5 (CH₃C═O), 202.9 (HC═O).

C9:0 ω-sophoroside aldehyde (bottom) (9-[(2′-O-β-D-glucopyranosyl-β-D-glucopyranosyl)oxy]nonanal): ¹³C NMR (100 MHz, DMSO-d₆): δ_{C} 22.0 (CH₂CH₂(C═O)H), 25.9 (CH₂(CH₂)₂), 29.0 (CH₂(CH₂)₂), 29.2 (CH₂(CH₂)₂), 29.3 (CH₂(CH₂)₂), 29.7 (CHOCH₂CH₂), 43.5 (CH₂(C═O)H), 61.35 (CH₂OH), 61.39 (CH₂OH), 69.1 (CHOCH₂CH₂), 70.2 (CHOC), 70.3 (CHOC), 75.3 (C²″H), 76.5 (C³′H), 76.6 (C³″H), 77.1 (CHOC), 77.5 (CHOC), 82.8 (C²′H), 101.8 (C¹′H), 104.6 (C¹″H), 203.9 (HC═O).

FIG. 14: The concentration of acetylated α,ω-bola sophorosides (g L⁻¹) (dots) plotted together with the evolution of the relative contents of C9:0 ω-sophoroside aldehydes (triangles) and C9:0 ω-sophorolipid carboxylic acid (diamonds) as a function of ozonolysis reaction time.

FIG. 15: General map of a vector comprising a knock-out cassette for deleting one of the seven genes of interest (GOI):a1, a2, a3, a4, a5, a6 or a7. Selection is done using the ura3 selection marker.

FIG. 16: Nucleotide and amino acid sequences of genes (a1-a7) encoding enzymes (A1-A7) identified as oxidizing enzymes responsible for ω-oxidation of long chain fatty alcohols and/or alkanes/alkenes. a1 (SEQ ID NO:100); A1(SEQ ID NO:101); a2 (SEQ ID NO:102); A2 (SEQ ID NO:103); a31 (SEQ ID NO:104); A3 (SEQ ID NO:105); a4 (SEQ ID NO:106); A4 (SEQ ID NO:107); a5 (SEQ ID NO:108); A5 (SEQ ID NO:109); a6 (SEQ ID NO:110); A6 (SEQ ID NO:111); a7 (SEQ ID NO:112); A7 (SEQ ID NO:113).

FIG. 17: UPLC-ELSD chromatograms of end samples of shake flask experiments for the A) Δcyp52M1 Δfao1 strain B) Δcyp52M1 Δfao1 Δa3 Δa4 strain and C) Δcyp52M1 Δfao1 Δa1 Δa3 Δa4 strain. All strains were fed with 1.8 (w/v) % oleyl alcohol. Respective masses determined with UPLC-MS are depicted above the respective peaks.

FIG. 18. Schematic representation of the cleavage of the double bond of one molecule of (acetylated) symmetrical C18:1 α,ω-bola glucoside using ozonolysis, resulting in the generation of (acetylated) C9:0 ω-glucoside aldehyde and (acetylated) C9:0 ω-glucolipid acid molecules and the comparison of 1H NMR spectra of (acetylated) glucosides as produced in example 1 (bottom spectrum) and the end sample after ozonolysis (top spectrum) in DMSO. The multiplet peak of the alkene protons at 5.33 ppm (CH═CH) from the C18 molecules present on the bottom spectrum disappeared upon ozonolysis on the top spectrum. The peaks of C9:0 ω-glucoside aldehydes at 9.65 ppm (HC═O) and 2.39 ppm (CH2(C═O)H) and C9:0 ω-glucolipid acid at 2.04 ppm (CH2(C═O)H) appeared on the spectrum of the end sample (top panel).

FIG. 19. Schematic representation of the cleavage of the double bond of (acetylated) symmetrical C18:1 α,ω-bola glycosides (sophorosides (A) and glycosides (B)) using a combination of a lipoxygenase and a hydroxyperoxide lyase, resulting in the generation of (acetylated) C9:0 ω-glycosides.

FIG. 20. Schematic representation of the cleavage of the double bond of (acetylated) symmetrical C18:1 α,ω-bola glycosides (sophorosides (A) and glycosides (B)) using a combination of a monooxygenase, an epoxide hydrolase and an alcohol dehydrogenase/monooxygenase, resulting in the generation of (acetylated) C9:0 ω-glycosides.

### DESCRIPTION OF INVENTION

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. Where reference is made to embodiments as comprising certain elements or steps, this encompasses also embodiments which consist essentially of the recited elements or steps.

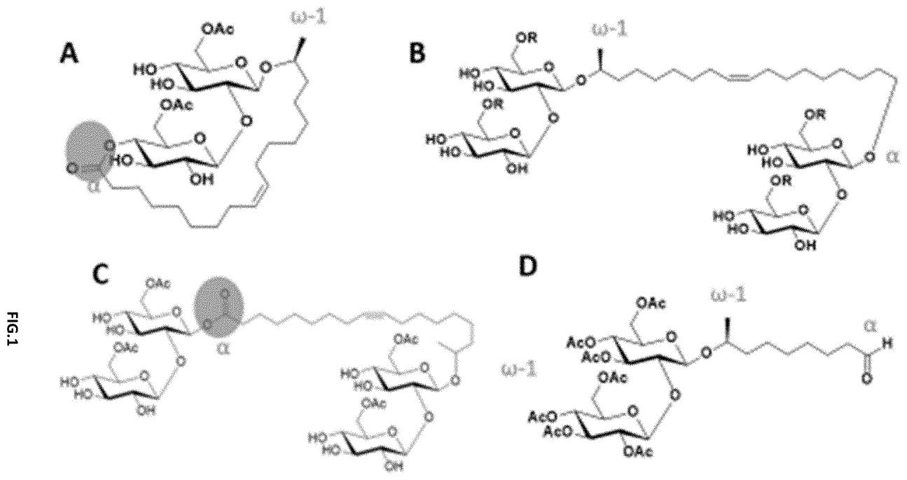

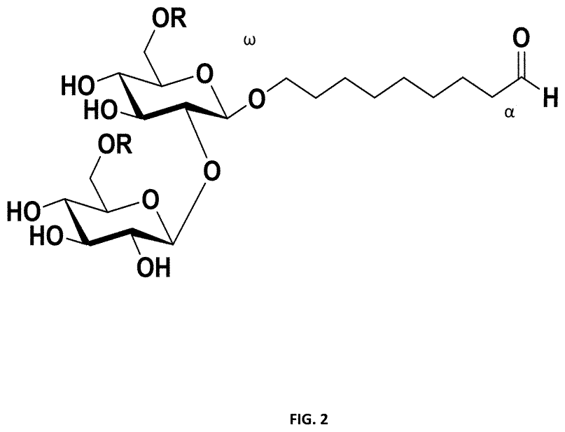

The present invention discloses the production of substantially uniform ω-glycosides (i.e. without or with minimal contaminating glycosides wherein a carbohydrate is connected to a functionalized aliphatic chain of carbons at another position than the w position such as, for example, ω-1 glycosides, ω-2 glycosides and/or ω-3 glycosides). ‘ω-glycosides’ or ‘omega-glycosides’ as disclosed herein (e.g. FIG. 2) are glycosides wherein the glycosidic bond connects the carbohydrate to a primary or terminal carbon atom of a functionalized aliphatic chain of carbons, whereas in ‘ω-1 glycosides’ or ‘omega-1 glycosides’, this glycosidic bond connects the carbohydrate to a secondary or subterminal carbon atom (see e.g. ω-sophoroside aldehydes in FIG. 2 versus ω-1 sophoroside aldehydes in FIG. 1D). The ω-glycosides disclosed herein are derived from so-called unsaturated α,ω-bola glycosides, which are produced with less than 10%, such as less than 9, 8, 7, 6, 5, 4, 3, 2, 1 or 0%, contaminating (unsaturated) α,ω-1-bola glycosides, α,ω-2-bola glycosides and/or α,ω-3-bola glycosides, via reaction conditions favoring the cleavage of at least one double bond of said unsaturated α,ω-bola glycosides. More specifically the latter reaction conditions may relate to ozonolysis in water or to the use of enzymes. More specifically and as a non-limiting example, the conditions that favor the selective production of C9:0 ω-sophoroside aldehyde, C9:0 ω-sophoroside alcohol and C9:0 ω-sophorolipid carboxylic acid, and derivatives thereof, without contaminating ω-1, ω-2 and/or ω-3 congeners are disclosed.

Hence, the present invention relates in a first aspect to a method to selectively produce ω-glycosides, in particular ω-glycosides which contain less than 10%, preferably less than 9, 8, 7, 6, 5, 4, 3, 2 or 1% ω-1 glycosides, ω-2 glycosides and/or ω-3 glycosides, comprising the steps of:
a. conversion of (a) suitable substrate(s) with a suitable microbial strain to produce a broth comprising unsaturated α,ω-bola glycosides which contains less than 10%, such as less than 9, 8, 7, 6, 5, 4, 3, 2 or 1%, of (unsaturated) α,ω-1-bola glycosides, α,ω-2-bola glycosides and/or α,ω-3-bola glycosides;
b. optionally purifying said unsaturated α,ω-bola glycosides from the broth of step a), and
c. subjecting said unsaturated α,ω-bola glycosides within said broth according to step a) to a reaction that breaks at least one unsaturated cleavable aliphatic bond within said unsaturated α,ω-bola glycosides, or, subjecting said unsaturated α,ω-bola glycosides which are purified according to step b) to a reaction that breaks at least one unsaturated cleavable aliphatic bond within said unsaturated α,ω-bola glycosides.

Cleavage of at least one unsaturated aliphatic bound present in the unsaturated α,ω-bola glycosides advantageously results in the production of two shorter chained ω-glycoside units with aldehyde (ω-glycoside aldehydes), alcohol (ω-glycoside alcohols) or carboxylic acid (ω-glycolipids) functionality depending on the process conditions, without the loss of an alkyl part, thus giving rise to improved yields.

A broth comprising unsaturated α,ω-bola glycosides which contains less than 10% of (unsaturated) α,ω-1-bola glycosides can also be referred to as a broth comprising more than 90%, such as more than 91, 92, 93, 94, 95, 96, 97, 98 or 99%, unsaturated α,ω-bola glycosides.

The term ‘glycoside’ generally refers to a molecule in which at least one carbohydrate molecule is covalently bound to another molecule via a glycosidic bond. The glycosides disclosed herein are more specifically covalently bound to a primary or terminal carbon of an aliphatic chain of carbons via a glycosidic bond, referred to as ‘omega-glycosides’ or ‘ω’-glycosides’, which terms are used interchangeably herein. Said aliphatic chain of carbons can be functionalized, preferably at the α position of said aliphatic chain, with for example, but without limitation, an aldehyde, a carboxyl or an alcohol functionality or derivatives thereof. Glycosides wherein a carbohydrate molecule is covalently bound via a glycosidic bond to an aliphatic chain of carbons that is functionalized at the α position with a carboxyl group are also referred to herein as ‘glycolipids’. Thus, in the framework of this invention, ‘glycolipids’ are compounds wherein one or more carbohydrate molecule(s) is/are covalently bound to a lipid molecule, wherein at least one of these lipids is an aliphatic carbon chain(s) of at least 6 carbon atoms that contains a carboxylic functionality, e.g. such as fatty acids.

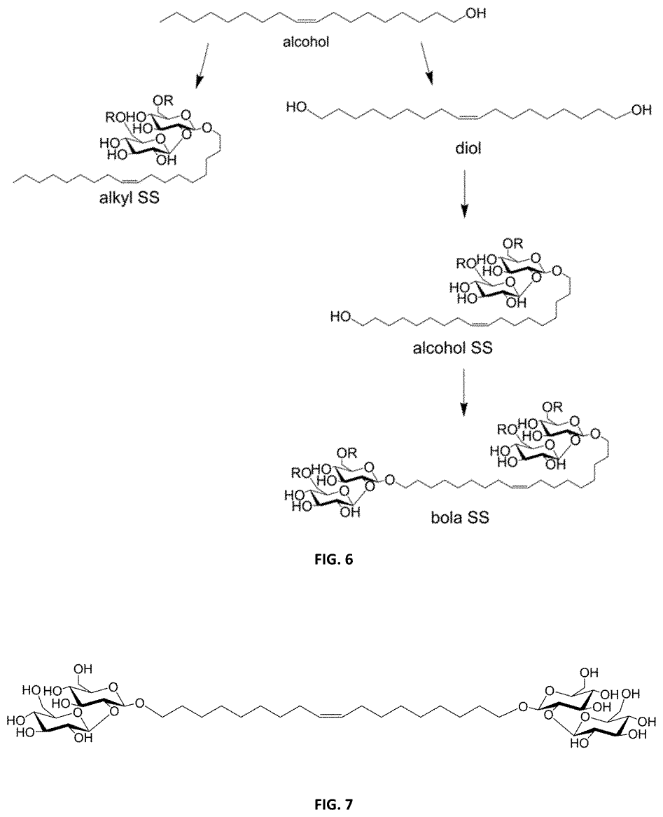

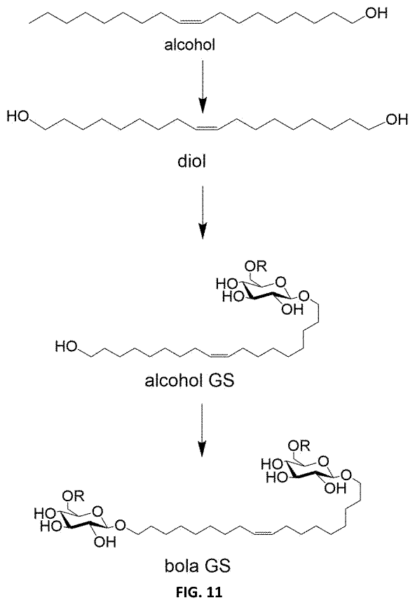

The term ‘bola glycoside’ as used herein refers to a glycoside molecule containing at least two carbohydrates both bound to a hydrophobic aliphatic linker, in particular an aliphatic chain of carbon atoms, connecting the two carbohydrates. The term ‘α,ω-bola glycoside’ refers to the fact that the two carbohydrate molecules are each connected to a ‘primary’ or ‘terminal’ carbon atom of the (functionalized) aliphatic chain of carbons (see e.g. FIGS. 6 and 11). In the case that one of the carbohydrates is connected to the aliphatic chain at another carbon atom than the terminal carbon atom, reference is made herein to an ‘α,ω-1-bola glycoside’ or an ‘β,ω-bola glycoside’, in case of linkage to the ‘subterminal’ carbon atom (see e.g. FIG. 1B). Likewise, the terms ‘α,ω-2-bola glycoside’ and ‘γ,ω-bola glycoside’ refer to the linkage of one of the carbohydrates to the third carbon atom of the aliphatic chain starting counting from each side, and the terms ‘α,ω-3-bola glycoside’ and ‘δ,ω-bola glycoside’ refer to the linkage of one of the carbohydrates to the fourth carbon atom of the aliphatic chain. The term ‘unsaturated’ as used in connection with α,ω-bola glycosides and α,ω-1-bola glycosides refers to the fact that at least one double bond is present in the aliphatic chain of carbons connecting both carbohydrate molecules. ‘Symmetrical unsaturated α,ω bola glycoside’ as used herein refers to the fact that one double bond is present in an aliphatic chain of carbon atoms connecting the two carbohydrates and that said double bond is situated in the middle of said hydrophobic linker.

Thus, an ω-glycoside as disclosed herein comprises an aliphatic chain of carbons, which can be functionalized at the α carbon and which contains a carbohydrate at the w carbon. Said ‘carbohydrate’ can be any carbohydrate known in the art, but is preferably glucose, sophorose, mannose, rhamnose, xylose, arabinose, trehalose, cellobiose or lactose. Hence, the present invention preferably relates to a method as described above wherein said ω-glycosides are, ω-glucosides, ω-sophorosides, ω-mannosides, ω-rhamnosides, ω-xylosides, ω-arabinosides, ω-trehalosides, ω-cellobiosides or ω-lactosides. The type of ω-glycoside that is produced can be tailored as known to the skilled person, e.g. through a proper selection of microbial strain (e.g. a natural sophorolipid producing yeast strain, e.g. Starmerella bombicola) and/or through further genetic modifications of the microbial strain. For example, upon replacing the glucosyltransferase UGTB1 by the glucosyltransferase UGT1 gene of U. maydis in Starmerella bombicola cellobiosides were produced instead of sophorosides (Roelants et al. 2013. Biotechnol Bioeng. 110:2494-2503).

In preferred embodiments of the method as described herein, the ω-glycosides are functionalized, in particular at the α position of the aliphatic chain, with an aldehyde group (ω-glycoside aldehyde), an alcohol group (ω-glycoside alcohol) or a carboxylic group (ω-glycolipid). Said ω-glycoside aldehydes, ω-glycoside alcohols and ω-glycolipids are encompassed within the term ‘ω-glycosides’ as used herein. In certain embodiments wherein the production of ω-glycoside aldehydes is desired, a catalase may be added to the broth (containing unsaturated α,ω-bola glycosides) that is subjected to the ozonolysis reaction or the enzymatic reaction.

In certain embodiments wherein the production of ω-glycolipids is desired, the ozonolysis reaction may be prolonged, preferably by at least 3 hours or by at least 1000%, preferably by at least 1200%, more preferably by at least 1400% of the calculated reaction time.

In other embodiments wherein the production of ω-glycolipids is desired, the method may further comprise a step of adding an oxidant (e.g. Oxone®) to the reaction medium following the ozonolysis reaction or the enzymatic reaction or to ω-glycoside aldehydes recovered from said reaction medium.

In certain embodiments wherein the production of ω-glycoside alcohols is desired, the method may further comprise a step of adding a reducing agent (e.g. picoline-borane) to the reaction medium following the ozonolysis reaction or the enzymatic reaction or to ω-glycoside aldehydes recovered from said reaction medium.

These ω-glycosides, in particular these ω-glycoside aldehydes, ω-glycoside alcohols and ω-glycolipids, can then be further derivatized through chemical derivatisation routes described in the art such as, but not limited to: acylation, alkylation, amidation, amination, arylation, biotinylation, carbamoylation, carbonylation, cycloaddition, coupling reaction, etherification, esterification, glycosylation, halogenation, metalation, metathesis, nitrile formation, olefination, oxidation, phosphinylation, phosphonylation, phosphorylation, quaternisation, rearrangement reaction, reduction, silylation, thiolation, thionation, or any combination thereof towards for example, but without limitation, ω-quaternary ammonium SLs (QASLs), ω-SS amine oxides, ω-SS amines, ω-bolamphiphile SSs, etc. such as for example described in the art for wild type SLs described by (Delbeke, 2016; Delbeke et al., 2018; Delbeke, Lozach, et al., 2016; Delbeke, Movsisyan, et al., 2015; Delbeke, Roelants, et al., 2016; Delbeke, Roman, et al., 2015; D. Develter & Fleurackers, 2008; Gross et al., 2013; Van Bogaert et al., 2011). The term ‘ω-glycosides’ as used herein also encompasses the derivatives of said ω-glycoside aldehydes, ω-glycoside alcohols and ω-glycolipids.

In embodiments of the method described herein, the method further comprises a step of subjecting the ω-glycosides obtained in step c), in particular the ω-glycoside aldehydes, ω-glycoside alcohols and/or ω-glycolipids obtained in step c), to a chemical derivatization route as described elsewhere herein, such as, for example, acylation, alkylation, amidation, amination, arylation, biotinylation, carbamoylation, carbonylation, cycloaddition, coupling reaction, etherification, esterification, glycosylation, halogenation, metalation, metathesis, nitrile formation, olefination, oxidation, phosphinylation, phosphonylation, phosphorylation, quaternisation, rearrangement reaction, reduction, silylation, thiolation and/or thionation.

Thus, also disclosed herein are methods for the production of derivates of ω-glycoside aldehydes, ω-glycoside alcohols and ω-glycolipids, said method comprising:
a. a conversion of (a) suitable substrate(s) with a suitable microbial strain to produce a broth comprising unsaturated α,ω-bola glycosides which contains less than 10%, such as less than 9, 8, 7, 6, 5, 4, 3, 2 or 1%, of (unsaturated) α,ω-1-bola glycosides, α,ω-2-bola glycosides and/or α,ω-3-bola glycosides;
b. optionally purifying said unsaturated α,ω-bola glycosides from the broth of step a);
c. subjecting said unsaturated α,ω-bola glycosides within said broth according to step a) to a reaction that breaks at least one unsaturated cleavable aliphatic bond within said unsaturated α,ω-bola glycosides, or, subjecting said unsaturated α,ω-bola glycosides which are purified according to step b) to a reaction that breaks at least one unsaturated cleavable aliphatic bond within said unsaturated α,ω-bola glycosides; and
d. subjecting the ω-glycosides obtained in step c) to a chemical derivatization route, preferably a chemical derivatization route selected from the group comprising acylation, alkylation, amidation, amination, arylation, biotinylation, carbamoylation, carbonylation, cycloaddition, coupling reaction, etherification, esterification, glycosylation, halogenation, metalation, metathesis, nitrile formation, olefination, oxidation, phosphinylation, phosphonylation, phosphorylation, quaternisation, rearrangement reaction, reduction, silylation, thiolation and/or thionation.

The methods described herein comprise a ‘conversion’ step of (a) suitable substrate(s) with a ‘suitable microbial strain’ to produce unsaturated α,ω-bola glycosides, with less than 10% contaminating (unsaturated) α,ω-1-bola glycosides, α,ω-2-bola glycosides and/or α,ω-3-bola glycosides.

Preferably, this ‘conversion’ is a ‘whole cell biocatalytic conversion’, i.e. a metabolic biological process executed by one or more microorganisms, wherein (bio)chemical changes are introduced in (a) suitable organic substrate(s) through the action of a set of enzymes present in a suitable microorganism, such as a bacterium or fungus. The ‘suitable substrate(s)’ thus get(s) converted into the final product (e.g. unsaturated α,ω-bola glycosides) through the expression and subsequent action of a set of genes encoding a set of enzymes by a ‘suitable microbial strain’.

Preferably ‘the suitable microbial strain’ is a natural SL producing yeast, in which at least (1) the gene(s) responsible for the (in S. bombicola mainly subterminal (ω-1)) hydroxylation of a fatty acid as a first step of natural SL biosynthesis is deleted (i.e. no wild type SL biosynthesis occurs anymore), in combination with the deletion of (2) the gene(s) responsible for conversion of fatty alcohols into fatty aldehydes. More specifically and respectively, the CYP52M1 (Van Bogaert et al., 2013) and FAO1 (F. Takahashi et al., 2016; Van Renterghem et al., 2018) genes in the case of S. bombicola, or their homologues in other SL producing yeast strains, have been deleted in the suitable microbial strain. In embodiments, the suitable microbial strain has no dysfunctional acetyltransferase and/or no dysfunctional lactonase.

The specific biosynthetic enzymes responsible for biosynthesis of unsaturated α,ω-bola glycosides utilize UDP-glucose precursors, (functionalized) aliphatic carbon chain precursors, preferably (saturated) fatty alcohols and also acetyl-CoA in case of acetylations.

For SL production, the SL producing yeast strains are typically cultivated on production media such as the one described by Lang et al., (2000) a.o. containing high levels of a suitable hydrophilic substrate, such as glucose, combined with a suitable hydrophobic substrate, such as rapeseed oil or oleic acid. The combined feeding of both a hydrophilic and a hydrophobic carbon source results in the highest SL productivity and is thus mostly preferred. The hydrophilic carbon source—even glucose—is catabolized and through gluconeogenesis, glucose is synthetized and activated towards UDP-glucose. The hydrophobic carbon source can be partly catabolized through β- and/or ω-oxidation, but can also be directly incorporated into SLs.

SL producing microorganisms such as S. bombicola, are also able to produce SLs when fed either on a hydrophilic or either on a hydrophobic carbon source (Cavalero & Cooper, 2003). This is due to the fact that the endogenous metabolic pathways allow the conversion of the hydrophilic carbon source e.g. glucose to fatty acids through the subsequent action of the glycolysis and fatty acid biosynthetic pathways. The first giving rise to acetyl-CoA, which is subsequently converted to fatty acids through the latter. Together with UPD-glucose (derived from the fed hydrophilic carbon source e.g. glucose), these (de novo) fatty acids are the building blocks of the SL biosynthetic pathway (Van Bogaert et al., 2013). The opposite is also true, e.g. the conversion of the hydrophobic carbon source e.g. fatty acids to glucose through the subsequent action of the β-oxidation and the gluconeogenic biosynthetic pathways. The first converting fatty acids into acetyl-coA, which can be further converted to glucose through the latter (Lin et al., 2001).

Accordingly, in embodiments, the suitable microbial strain is fed with a hydrophilic carbon source (preferably glucose) and/or a hydrophobic carbon source (preferably a(n) (unsaturated) primary fatty alcohol, preferably oleyl alcohol), preferably a hydrophilic carbon source and a hydrophobic carbon source, to convert said carbon source(s) in step a) into a broth comprising unsaturated α,ω-bola glycosides which contains less than 10%, such as less than 9, 8, 7, 6, 5, 4, 3, 2 or 1%, of (unsaturated) α,ω-1-bola glycosides, α,ω-2-bola glycosides and/or α,ω-3-bola glycosides.

In embodiments, the suitable microbial strain as described herein has additional genetic modifications allowing the use of either a hydrophilic carbon source such as glucose (without a hydrophobic carbon source fed) or either a hydrophobic carbon source such as fatty acids or vegetable oils (without a hydrophilic carbon source fed) for the production of unsaturated α,ω-bola glycosides, with less than 10% α,ω-1-bola glycosides, α,ω-1-bola glycosides and/or α,ω-3 bola glycosides.

In particular, the suitable microbial strain described above can be further engineered with methods described in the art allowing the adapted strain to use fatty acids, e.g. oleic acid, or plant oils, e.g. high oleic sunflower oil (HOSO), as the hydrophobic substrate without a hydrophilic carbon source fed, according to the method described above. This can be achieved by the (over)expression of two enzymes efficiently converting the fed plant oils/fatty acids into fatty alcohols in such suitable strain. Examples of such enzymes are e.g. carboxylic acid reductase enzymes (Kalim Akhtara et al., 2013) converting fatty acids into fatty aldehydes, which can subsequently be converted into fatty alcohols e.g. by the simultaneous expression of an aldehyde reductase or an aldehyde decarboxylase (Fatma et al., 2016; Kang & Nielsen, 2017). Fully saturated fatty acids can also be fed and converted into unsaturated fatty acids through the additional expression of a fatty acid desaturase (Cifre et al., 2013). Such further engineering in the abovementioned suitable strain would allow the resulting strain to be fed exclusively with fatty acids and/or fatty acid containing oils/fats, without simultaneously feeding a hydrophilic carbon source, still allowing the production of unsaturated α,ω-bola glycosides with less than 10% contaminating α,ω-1-bola glycosides, α,ω-2-bola glycosides and/or α,ω-3-bola glycosides. The fatty acids will namely be converted into fatty alcohols as described above, but also into acetyl-CoA, through the—oxidation, which will then again be converted into UDP-glucose through gluconeogenesis. Together these are—as mentioned above—the required precursors to enter the biosynthetic pathway towards α,ω-bola glycosides. However, such new engineered strain would—as defined above—preferably be fed with a combination of a hydrophilic carbon source (preferably glucose) with a hydrophobic carbon source (preferably a(n) (unsaturated) primary fatty alcohol, preferably oleyl alcohol) giving rise to higher productivities.

The suitable microbial strain described above can also be used to produce unsaturated α,ω-bola glycosides with less than 10% contaminating (unsaturated) α,ω-1 bola glycosides, α,ω-2-bola glycosides and/or α,ω-3-bola glycosides using a hydrophilic carbon source, such as glucose, glycerol, sucrose, fructose, maltodextrins, starch hydrolysates, lactose, mannose, xylose, arabinose, molasses, etc. or mixtures thereof without feeding a hydrophobic carbon source. The described strain can use its endogenous pathways to convert the hydrophilic carbon source, such as glucose, into fatty acids through glycolysis (giving rise to acetyl-CoA) followed by de novo fatty acid biosynthesis. These fatty acids can subsequently be further converted into fatty alcohols due to the expression of the genes/enzymes described above. Together with the UDP-glucose, derived from the fed hydrophilic carbon source, these fatty alcohols will enter the biosynthetic pathway towards unsaturated α,ω-bola glycosides.

In embodiments, the suitable microbial strain as described herein can be further engineered according to methods described in the art to allow the feeding of hydrophobic substrates selected from alkanes and/or alkenes to allow the synthesis of unsaturated α,ω-bola glycosides with less than 10% (unsaturated) α,ω-1-bola glycosides, α,ω-2-bola glycosides and/or α,ω-3-bola glycosides. In particular, the suitable microbial strain can be further engineered with the additional (over)expression of optionally an (endogenous) desaturase converting an alkane into an alkene, and an (endogenous) oxidizing enzyme e.g. a CYP52M1 enzyme allowing the oxidation of the alkene to the corresponding unsaturated fatty alcohol, which can then be further converted to the unsaturated α,ω-bola glycosides as described above.

In embodiments, (a) ‘suitable substrate(s)’ refers to a hydrophilic substrate such as glucose and/or a hydrophobic substrate, preferably a combination of a hydrophilic substrate such as glucose and a hydrophobic substrate, more preferably a combination of a hydrophilic substrate such as glucose and an unsaturated, hydrophobic substrate such as a fatty alcohol, having an aliphatic tail chain length of at least 6 carbons.

Non-limiting examples of (a) hydrophilic substrate(s) are substrates containing carbohydrates such as glucose, glycerol, sucrose, fructose, maltodextrins, starch hydrolysates, lactose, mannose, xylose, arabinose, molasses, etc. or mixtures thereof. Non-limiting examples of (a) hydrophobic substrate(s) are fatty alcohols, fatty acids, plant- or animal oils/fats, saturated and/or unsaturated hydrocarbons such as linear alkanes, alkenes, etc. and/or mixtures thereof. Preferably fatty alcohols are used, of which non-limiting examples are hexanol, heptanol, octanol, nonanol, decanol, undecanol, dodecanol, tridecanol, tetradecanol, pentadecanol, hexadecanol, palmitoleyl alcohol, heptadecanol, octadecanol, octadecenol, isostearyl alcohol, nonadecanol, icosanol, heneicosanol, docosanol, docosenol and/or mixtures thereof. In further embodiments, the (fatty) alcohol has maximal one hydroxyl group (i.e. is a monoalcohol), preferably the (fatty) alcohol is a primary fatty alcohol. In further embodiments, the (fatty) alcohol is not a diol. More preferably primary, unsaturated, linear fatty alcohols are used in which the double bond within said fatty alcohol can be situated between any pair of following carbon atoms within said alcohol, but is for the even molecules preferably situated in the ‘middle’ of said alcohol. In the last case, the thereof derived unsaturated α,ω-bola glycosides will be ‘symmetrical’ i.e. the double bond is present in the middle of the hydrophobic aliphatic linker and flanked at both sides with a carbohydrate. In case a double bond is not present in the fed alcohol, such unsaturation can be introduced trough the action of (an) endogenous ‘desaturase’ enzyme(s) present in the microorganism. Alternatively, an heterologous ‘desaturase’ gene/enzyme can be expressed in the suitable strain. Hence, in further embodiments, a suitable hydrophobic substrate is an unsaturated fatty alcohol having an aliphatic chain length of at least 6 carbons preferably an unsaturated fatty alcohol having a chain length of 18 carbon atoms with the double bond present at position C9 (i.e. in the middle of the C18 linker). In embodiments of the methods of the present invention said unsaturated α,ω-bola glycosides are fully symmetrical. In embodiments of the methods of the present invention, a fatty alcohol is metabolized by a suitable microorganism in order to convert said fatty alcohol into unsaturated α,ω-bola glycosides without the formation of undesired and contaminating α,ω-1-bola glycosides, α,ω-2-bola glycosides and/or α,ω-3-bola glycosides.

In embodiments, the suitable microbial strain is not fed with a diol.

In an optional step of the method described herein, the unsaturated α,ω-bola glycosides can be purified—by any method known in the art—for example by a microfiltration to remove the microbial cells followed by a two-step ultrafiltration process to remove both large and small size contaminants from the bioprocess broth in which these molecules are made, resulting in a purified α,ω-bola glycoside liquid stream, which can optionally be freeze dried (Roelants et al., 2016; Van Renterghem et al., 2018) In a further step of the method described herein, the unsaturated α,ω-bola glycosides, optionally purified, present in the bioprocess broth are subjected to reaction conditions favoring cleavage of at least one double bond in the unsaturated α,ω-bola glycosides. A non-limiting example of such reaction condition is an ozonolysis reaction, which results in the oxidative cleavage of the double bond(s) of said unsaturated α,ω-bola glycoside, giving rise to the formation of two shorter chained ω-glycoside molecules per bola glycoside molecule. In embodiments, two identical omega-glycosides per bola glycoside can be generated in case said unsaturated α,ω-bola glycosides are symmetrical. As described elsewhere herein, said shorter chained omega-glycosides can be further functionalized—at the α position—with an aldehyde group, an alcohol group or a carboxylic acid group. These shorter chained ω-glycosides with an aldehyde (sophoroside aldehyde), alcohol (sophoroside alcohol) or acidic (sophorolipid) group at the α position can be made selectively by varying the specific process conditions for cleaving the double bond of the unsaturated α,ω-bola glycosides (Delbeke, 2016; Delbeke, Roman, et al., 2015; Lorer, 2017).

Suitable microorganisms to produce unsaturated α,ω-bola glycosides (which can be symmetrical) are for example mutated fungal strains having a dysfunctional cytochrome P450 monooxygenase that is responsible for the first hydroxylation step in the SL biosynthetic pathway, in particular CYP52M1 or a homologue thereof, and a dysfunctional fatty alcohol oxidase that is responsible for the conversion of fatty alcohols to the corresponding fatty aldehydes (and so further to fatty acids by the action of an aldehyde dehydrogenase), in particular FAO1 or a homologue thereof, or mutated fungal strains having a dysfunctional cytochrome P450 monooxygenase that is responsible for the first hydroxylation step in the SL biosynthetic pathway, in particular CYP52M1 or a homologue thereof, a dysfunctional fatty alcohol oxidase, that is responsible for the conversion of fatty alcohols to the corresponding fatty aldehydes (and so further to fatty acids by the action of an aldehyde dehydrogenase), in particular FAO1 or a homologue thereof, and a dysfunctional glucosyltransferase that is responsible for the second glucosylation step in the SL biosynthetic pathway, in particular UGTB1 or a homologue thereof, wherein said fungal strain is preferably a yeast selected from the group consisting of Starmerella bombicola (previously Candida), Starmerella apicola (Gorin et al., 1961) (previously Candida), which was initially identified as T. magnolia, C. bombicola (Spencer et al., 1970), Wickerhamiella domericqiae (Chen et al., 2006), Pseudohyphozyma bogoriensis (previously Rhodotorula or Candida) (Tulloch et al., 1968), Starmerella batistae (Konishi et al., 2008) (previously Candida), Starmerella floricola (Imura et al., 2010) (previously Candida), Candida riodocensis, Candida tropicalis, Starmerella stellata (previously Candida) and Candida sp. NRRL Y-27208 (Kurtzman et al., 2010), Starmerella kuoi (Kurtzman, 2012) (previously Candida), Candida gropengiesseri, Candida magnoliae, Candida Antarctica, Pseudozyma antarctica, Candida tropicalis, Candida lipolytica and any other SL producing strain (of the Starmerella clade).

The term “a mutated fungal strain” relates to a fungal strain as defined above wherein said strain is mutated so that the enzymes CYP52M1 or a homologue thereof and FAO1 or a homologue thereof are non- or dysfunctional. With regard to CYP52M1 or its homologue(s), this means that no ω-1 (or ω-2) hydroxylation of (de novo produced) fatty acids, but neither of fatty alcohols can occur anymore by this enzyme, which results in an absence of SLs produced by such strain (Van Bogaert et al., 2013). With regards to FAO1 or its homologue(s) this means that no oxidation of the OH group present on the fatty alcohol precursors (of the biosynthetic pathway towards α,ω-bola glycosides) towards the corresponding aldehyde can occur anymore, which aldehyde would then be further oxidized into a fatty acid, which would on its turn be hydroxylated and be incorporated into SLs. More specifically, the route towards SL production from (de novo) fatty acids and from fatty alcohols (converted into fatty acids) is blocked. In theory and based on the information in the art, this should give rise to selective production of alkyl sophorosides (free from contaminating SLs) upon feeding this strain with (a) fatty alcohol(s) and a hydrophilic carbon source such as glucose. Surprisingly this strain instead produces unsaturated α,ω-bola sophorosides when fed with (a) fatty alcohol(s) and glucose together with low amounts of alkyl sophorosides. Advantageously, these strains selectively produce unsaturated α,ω-bola sophorosides, i.e. without or with minimal fatty acid derived sophorolipids such as acidic, lactonic or bola sophorolipids that are produced by the corresponding strains that are not mutated in CYP52M1 or its homologue. Accordingly, the ω-glycosides that are obtained in the subsequent steps of the methods described herein, will also not or minimally be contaminated with these sophorolipids or derivatives of these sophorolipids.

The term ‘dysfunctional’ means in general a gene or protein which is not functioning ‘normally’, and/or, has an absent or impaired function. The term thus refers to a gene or protein which is: a) not functional because it is not present, b) still present but is rendered non-functional or c) which is present but has a weakened or reduced function such as a gene or protein that has retained a function or activity that is less than 90%, 80, 70%, 60% or 50%, 40% or 30%, preferably less than 20%, more preferably less than 10%, even more preferably less than 5% such as less than 4%, 3%, 2% or 1% of the function or activity of the corresponding wild-type gene or protein. The term ‘dysfunctional’ specifically refers to a gene having lost its capability to encode for the fully functional enzymes CYP52M1 or its homologue(s) and FAO1 or its homologue(s), or polypeptides/proteins having lost its CYP52M1 and FAO1 activity, either completely or partially. ‘Partially’ means that the activity of the latter enzymes—measured by any method known in the art—is significantly lower (p<0.05) when compared to the activity of the wild-type counterparts of said enzymes, such as at least 10%, at least 20%, at least 30%, at least 40%, at least 50%, at least 60% or at least 70%, preferably at least 80%, more preferably at least 90%, even more preferably at least 95% such as at least 96%, 97%, 98% or 99% lower compared to the activity of the wild-type counterparts of said enzymes.

A ‘dysfunctional’ nucleic acid molecule as defined above can be obtained by mutation or by any known means to silence the transcription or translation of said nucleic acid. The latter comprises the insertion of a nucleic acid fragment, a marker gene or any other molecule in the target gene, a mutation or removal of the target gene, the usage of specific siRNAs, miRNAs or combinations thereof, or any other means known to a skilled person.

The term ‘mutation’ refers to a spontaneous mutation and/or to an induced and/or directed mutation in the genome of said fungal strain. Said mutation can be a point mutation, deletion, frameshift, insertion or any other type of mutation.

Similarly, a ‘dysfunctional’ polypeptide as defined above can be obtained by any (small) compound or other means to weaken or disrupt the function of the target genes described herein. Means to silence the transcription or translation or means to disrupt the function of the target genes of the present invention or means to disrupt the function of a necessary regulator/activator protein of the target genes comprise the usage of any molecule such as—but not limited to—an antibody, an amino acid, a peptide, a small molecule, an aptamer, a ribozyme, an oligoribonucleotide sequence such a dsRNA used to initiate RNA interference (RNAi) or an anti-sense nucleic acid. Such a molecule is thus capable to bind on a target protein or an activator/regulator protein thereof, or, is capable to interfere with the cellular synthesis of the target enzyme or of an activator/regulator thereof by—for example—binding and degrading mRNA's encoding for a target protein or an activator/regulator thereof.

A ‘dysfunctional’ CYP52M1 or its homologue(s) and FAO1 or its homologue(s) refers to an enzyme with reduced activity, obtained by any method known by the person skilled in the art. Non-limiting examples of said methods are the introduction of point mutations, the usage of truncated or mutated enzymes, the usage of inhibitors or antibodies, and any of the methods described above.

The term ‘dysfunctional’ thus also refers to the absence of the specific genes mentioned above (cyp52M1 and fool) in the genome of the applied fungal strain.

The genes and their encoded enzymes CYP52M1 and FAO1 and UGTB1 are well known in the art and are—for example—described in patent WO2011154523 (Soetaert et al., 2010) (CYP52M1, UGTB1) and in Takahashi et al., (2016) (FAO1).

This latter strain can be made by any method known in the art and as is described above.

In further embodiments, the mutated fungal strain as described above further comprises a dysfunctional glucosyltransferase that is responsible for the second glucosylation step in the sophorolipid/sophoroside biosynthetic pathway, in particular UGTB1 or a homologue thereof.

The term ‘A glucosyltransferase that is responsible for the second glucosylation step in the SL biosynthetic pathway’ is described in detail in WO2011154523 (Soetaert et al., 2010). Indeed WO2011154523 discloses that there is a first glycosylation (see example 2 of WO2011154523) and a second glycosylation step (see example 3 of WO2011154523) in the SL pathway wherein a ‘first’ (i.e. UGTA1 or a homologue thereof) and a ‘second’ glycosyltransferase (i.e. UGTB1 having Genbank Accession number HM440974 and also described in detail in Saerens, et al., (2011) or a homologue thereof), are involved.

Any other microbial host strain that is modified according to methods described in the art to express the required enzymatic steps towards the production of unsaturated α,ω-bola glycosides (Van Bogaert et al., 2013, 2016; Van Renterghem et al., 2018) and free of enzymatic activities resulting in the biosynthesis of any one or more of α,ω-1 or α,ω-2 or α,ω-3 (unsaturated) bola glycosides can be used in the methods of the present invention.

In embodiments, said unsaturated α,ω-bola glycosides can be (tetra-, tri-, di-, mono- and/or non-) acetylated resulting in di- or mono-acetylated derived ω-glycosides after breaking the double bond.

With the term ‘acetylated’ is specifically meant glycosides which contain an ‘acetyl’ functionality on position 6′ or 6″ of the sugar moieties present in said bola glycosides. The term ‘acetylation’ (or ethanoylation) more generally describes a reaction that introduces an acetyl functional group into a chemical compound resulting in an acetoxy group i.e. the substitution of an acetyl group for an active hydrogen atom. A reaction involving the replacement of the hydrogen atom of a hydroxyl group with an acetyl group (CH₃ CO) yields a specific ester, the acetate.

In particular embodiments, the ω-glycosides that are produced are C9:0 ω-sophorosides or C9:0 ω-glucosides.

In embodiments, the present invention relates to a method as described above wherein during ozonolysis a protic nucleophilic, preferably water, is used as a solvent in order to overcome safety concerns with ozonolysis and to increase the green nature of the process.

In particular embodiments, a method is provided for the production of C9:0 ω-sophorosides, said method comprising:
a. conversion of glucose and oleyl alcohol to produce a broth comprising symmetrical C18:1 α,ω-bola sophorosides which contain less than 10% of C18:1 α/ω-1 bola sophorosides and/or C18:1 α/ω-2 bola sophorosides with a S. bombicola strain wherein the gene cyp52M1 and the fool gene have been knocked out;
b. optionally purifying said symmetrical C18:1 α,ω-bola sophorosides;
c. subjecting said symmetrical C18:1 α,ω-bola sophorosides within said broth according to step a) to an ozonolysis reaction, preferably an ozonolysis reaction in water; and
d. optionally subjecting the C9:0 ω-sophorosides obtained in step c) to a suitable chemical derivatization route, preferably a chemical derivatization route selected from the group comprising: acylation, alkylation, amidation, amination, arylation, biotinylation, carbamoylation, carbonylation, cycloaddition, coupling reaction, etherification, esterification, glycosylation, halogenation, metalation, metathesis, nitrile formation, olefination, oxidation, phosphinylation, phosphonylation, phosphorylation, quaternisation, rearrangement reaction, reduction, silylation, thiolation and/or thionation.

In particular embodiments, a method is provided for the production of C9:0 ω-glucosides said method comprising:
a. conversion of glucose and oleyl alcohol to produce a broth comprising symmetrical C18:1 α,ω-bola glucosides which contain less than 10% of symmetrical C18:1 α,ω-1 bola glucosides and/or C18:1 α,ω-2 bola glucosides with a S. bombicola strain wherein the gene cyp52M1, fool gene and ugtB1 gene have been knocked out,
b. optionally purifying said symmetrical C18:1 α,ω-bola glucosides,
c. subjecting said symmetrical α,ω-bola glucosides within said broth according to step a) to an ozonolysis reaction, preferably an ozonolysis reaction in water,
d. optionally subjecting the C9:0 ω-glucosides obtained in step c) to a suitable chemical derivatization route, preferably a chemical derivatization route selected from the group comprising: acylation, alkylation, amidation, amination, arylation, biotinylation, carbamoylation, carbonylation, cycloaddition, coupling reaction, etherification, esterification, glycosylation, halogenation, metalation, metathesis, nitrile formation, olefination, oxidation, phosphinylation, phosphonylation, phosphorylation, quaternisation, rearrangement reaction, reduction, silylation, thiolation and/or thionation.

The invention also provides a method to produce glycosides, which are not functionalized at the α position and thus characterized by a non-functionalized methyl function at the α position. Said glycosides which are not functionalized at the α position are referred to herein as ‘alkyl glycosides’. An ‘alkyl glycoside’ as described herein thus refers to a molecule in which at least one carbohydrate molecule is covalently bound via a glycosidic bond to an aliphatic chain of carbons that is not functionalized at the α position. At least one carbohydrate molecule is bound to a primary or terminal carbon of the aliphatic chain of carbons (i.e. ω-alkyl glycosides) or to a secondary or subterminal carbon atom of the aliphatic chain of carbons (e.g. ω-1-alkyl glycosides, ω-2-alkyl glycosides). In preferred embodiments, the alkyl glycosides are ω-alkyl glycosides. The alkyl glycosides can be di-, mono- and/or non-acetylated (i.e. the alkyl glycoside can contain an acetyl functionality on 6′ or 6″ of the sugar moiety present in said alkyl glycoside. Non-limiting examples of alkyl glycosides are alkyl sophorosides and alkyl glucosides.

Provided herein is an improved method to produce alkyl glycosides in increased ratio's. In particular, a method is provided to produce alkyl glycosides, comprising the step of a conversion of (a) suitable substrate(s) with a suitable microbial strain to produce a broth comprising alkyl glycosides. ‘Suitable substrate(s)’ are the substrates that are suitable for use in the methods to selectively produce ω-glycosides as described elsewhere herein, a preferred substrate is a primary fatty alcohol. For the production of ω-1 alkyl glycosides and ω-2 alkyl glycosides, the strains can be fed with respectively, secondary or tertiary fatty alcohols. ‘Suitable strains’ are the strains that are suitable for use in the methods to selectively produce ω-glycosides as described elsewhere herein, more specifically SL producing strains as described above, which are mutated in the CYP52M1 gene or its homologue(s) and mutated in the FAO1 gene or its homologue (for the production of alkyl sophorosides); or which are mutated in the CYP52M1 gene or its homologue(s) and mutated in the FAO1 gene and mutated in the UGTB1 gene or its homologue(s) (for the production of alkyl glucosides). Preferably, the ‘suitable strains’ are additionally mutated in at least one, preferably at least two such, more preferably at least three, endogenous gene encoding an ‘oxidizing gene/enzyme’ responsible for ω-oxidation of long chain fatty alcohols and/or alkanes/alkenes, more particularly at least one, preferably at least two, more preferably as at least three, endogenous gene encoding an oxidizing enzyme responsible for ω-oxidation of long chain fatty alcohols and/or alkanes/alkenes has additionally been mutated so that said oxidizing enzyme is dysfunctional or nonfunctional, preferably nonfunctional, resulting in increased ratios of alkyl glycosides. Said oxidizing enzyme(s) are responsible for oxidation of primary fatty alcohols giving rise to the generation of long chain α,ω-fatty diols, which can be further incorporated into α,ω-bola glycosides. In embodiments, at least one, preferably at least two, more preferably as at least three, oxidizing enzyme is selected from the group comprising: A1 comprising the amino acid sequence set forth in SEQ ID NO:101 or a homologue thereof, A2 comprising the amino acid sequence set forth in SEQ ID NO:103 or a homologue thereof, A3 comprising the amino acid sequence set forth in SEQ ID NO:105 or a homologue thereof, A4 comprising the amino acid sequence set forth in SEQ ID NO:107 or a homologue thereof, A5 comprising the amino acid sequence set forth in SEQ ID NO:109 or a homologue thereof, A6 comprising the amino acid sequence set forth in SEQ ID NO:111 or a homologue thereof and A7 comprising the amino acid sequence set forth in SEQ ID NO: 113 or a homologue thereof.

In preferred embodiments, the suitable strain is additionally mutated in at least the gene a3 comprising the nucleotide sequence set forth in SEQ ID NO:104 (encoding A3 comprising the amino acid sequence set forth in SEQ ID NO:105) or a homologue thereof and the gene a4 comprising the nucleotide sequence set forth in SEQ ID NO:106 (encoding A4 comprising the amino acid sequence set forth in SEQ ID NO:107) or a homologue thereof. In preferred embodiments, the suitable strain is additionally mutated in at least the gene a3 comprising the nucleotide sequence set forth in SEQ ID NO:104 (encoding A3 comprising the amino acid sequence set forth in SEQ ID NO:105) or a homologue thereof, the gene a4 comprising the nucleotide sequence set forth in SEQ ID NO:106 (encoding A4 comprising the amino acid sequence set forth in SEQ ID NO:107) or a homologue thereof, and the gene a1 comprising the nucleotide sequence set forth in SEQ ID NO:100 (encoding A1 comprising the amino acid sequence set forth in SEQ ID NO:101) or a homologue thereof. The additional mutations in at least the genes a3 and a4 (to have at least dysfunctional or nonfunctional A3 and A4) (selectively) increased alkyl glycoside production; the additional mutations in at least the genes a3, a4 and a1 (to have at least dysfunctional or nonfunctional A3, A4 and A1) further increased selective alkyl glycoside production (i.e. with less bolaform sophoraside co-production).

In embodiments, the suitable strain is additionally mutated in the gene a1 comprising the nucleotide sequence set forth in SEQ ID NO:100 (encoding A1 comprising the amino acid sequence set forth in SEQ ID NO:101) or a homologue thereof, the gene a2 comprising the nucleotide sequence set forth in SEQ ID NO:102 (encoding A2 comprising the amino acid sequence set forth in SEQ ID NO:103) or a homologue thereof, the gene a3 comprising the nucleotide sequence set forth in SEQ ID NO:104 (encoding A3 comprising the amino acid sequence set forth in SEQ ID NO:105) or a homologue thereof, the gene a4 comprising the nucleotide sequence set forth in SEQ ID NO:106 (encoding A4 comprising the amino acid sequence set forth in SEQ ID NO:107) or a homologue thereof, the gene a5 comprising the nucleotide sequence set forth in SEQ ID NO:108 (encoding A5 comprising the amino acid sequence set forth in SEQ ID NO:109) or a homologue thereof, the gene a6 comprising the nucleotide sequence set forth in SEQ ID NO:110 (encoding A6 comprising the amino acid sequence set forth in SEQ ID NO:111) or a homologue thereof, and the gene a7 comprising the nucleotide sequence set forth in SEQ ID NO:112 (encoding A7 comprising the amino acid sequence set forth in SEQ ID NO: 113 or a homologue thereof.

A further aspect relates to use of an enzyme A1 comprising the amino acid sequence set forth in SEQ ID NO:101 or a homologue thereof, an enzyme A3 comprising the amino acid sequence set forth in SEQ ID NO:105 or a homologue thereof or an enzyme or an enzyme A4 comprising the amino acid sequence set forth in SEQ ID NO:107 or a homologue thereof for the production of diols, preferably α,ω-diols.

A related aspect is directed to a method for the production of diols, preferably α,ω-diols, said method comprising contacting a fatty alcohol, preferably a primary fatty alcohol, with an enzyme A1 comprising the amino acid sequence set forth in SEQ ID NO:101 or a homologue thereof, an enzyme A3 comprising the amino acid sequence set forth in SEQ ID NO:105 or a homologue thereof or an enzyme or an enzyme A4 comprising the amino acid sequence set forth in SEQ ID NO:107 or a homologue thereof, so as to produce diols, preferably α,ω-diols.

Some methods described herein relate to the production of diols, preferably α,ω-diols, using a (purified) oxidizing enzyme responsible for oxidation of primary fatty alcohols disclosed herein, in particular an enzyme A1, A3 or A4, and a fatty alcohol substrate, preferably a primary fatty alcohol substrate. For example, a host cell can be genetically engineered to (over)express an oxidizing enzyme responsible for oxidation of primary fatty alcohols as disclosed herein, in particular an enzyme A1, A3 or A4. The recombinant host cell can be cultured under conditions sufficient to allow (over)expression of the oxidizing enzyme. Cell-free extracts can then be generated using known methods. For example, the host cells can be lysed using detergents or by sonication. The overexpressed oxidizing enzymes can be purified using known methods, or the cell-free extracts can be used as such for the production of diols. The host cells can also be genetically engineered to (over)express an oxidizing enzyme responsible for oxidation of primary fatty alcohols as disclosed herein, in particular an enzyme A1, A3 or A4, and to secrete said oxidizing enzyme into the culture medium. A secretion signal sequence can be operably linked to the nucleic acid encoding the oxidizing enzyme to this end. In this connection, “operably linked” denotes that the sequence encoding the secretion signal peptide and the sequence encoding the polypeptide to be secreted are connected in frame or in phase, such that upon expression the signal peptide facilitates the secretion of the polypeptide so-linked thereto. The secreted oxidizing enzymes can then be separated from the culture medium and optionally purified using known methods without the need for obtaining cell-free extracts.

Next, fatty alcohols, preferably primary fatty alcohols, can be added to the cell-free extracts or (purified) oxidizing enzymes and maintained under conditions to allow terminal hydroxylation of the fatty alcohol substrate or the primary fatty alcohol substrate, to produce respectively, diols or α,ω-diols. The diols or α,ω-diols can then be separated and purified using known techniques.

Other methods described herein relate to the production of diols, preferably α,ω-diols, which method comprises culturing a genetically engineered host cell in a culture medium so as to allow the production of diols, preferably α,ω-diols, wherein said host cell is genetically engineered to (over)express a gene encoding an oxidizing enzyme responsible for oxidation of primary fatty alcohols disclosed herein (i.e. wherein said genetically engineered host cell comprises a (recombinant) nucleic acid encoding an oxidizing enzyme responsible for oxidation of primary fatty alcohols disclosed herein), in particular the gene a1 comprising the nucleotide sequence set forth in SEQ ID NO:100 (encoding A1 comprising the amino acid sequence set forth in SEQ ID NO:101) or a homologue thereof, the gene a3 comprising the nucleotide sequence set forth in SEQ ID NO:104 (encoding A3 comprising the amino acid sequence set forth in SEQ ID NO:105) or a homologue thereof, or the gene a4 comprising the nucleotide sequence set forth in SEQ ID NO:106 (encoding A4 comprising the amino acid sequence set forth in SEQ ID NO:107) or a homologue thereof.

Non-limiting examples of host cells suitable for use in the methods for the production of diols described herein include oleaginous fungi such as yeasts from the genera Yarrowia (e.g. Yarrowia lipolytica), Candida (e.g. Candida tropicalis), Rhodotorula, Rhodosporidium, Cryptococcus, Trichosporon, and Lipomyces, and natural sophorolipid producing fungal strains as described elsewhere herein, e.g. Starmerella (Candida) bombicola, Starmerella (Candida) apicola, Candida magnoliae, Candida gropengiesseri, Starmerella (Candida) batistae, Starmerella (Candida) floricola, Candida riodocensis, Candida tropicalis, Starmerella (Candida) stellata, Starmerella (Candida) kuoi, Candida sp. NRRL Y-27208, Pseudohyphozyma (Rhodotorula, Candida) bogoriensis sp., Wickerhamiella domericqiae and sophorolipid-producing strains of the Starmerella clade, wherein said natural sophorolipid producing fungal strains are preferably mutated to have (a) dysfunctional glucosyltransferase(s) that is/are responsible for the glycosylation step(s) in the sophorolipid biosynthetic pathway, e.g. a dysfunctional UGTA1 or a homologue thereof and a dysfunctional UGTB1 or a homologue thereof.

The genetically engineered or recombinant host cells are cultured under conditions suitable for the production of diols, preferably α,ω-diols, by the host cells. More particularly this implies “conditions sufficient to allow (over)expression” of the gene encoding the oxidizing enzyme disclosed herein, in particular the gene a1, a3 or a4, which means any condition that allows the host cell to (over)produce an oxidizing enzyme disclosed herein as described herein. The conditions suitable for the production of diols, preferably α,ω-diols, may further imply cultivating the host cells in a culture medium which comprises at least one fatty alcohol substrate, preferably at least one primary fatty alcohol substrate, which is terminally hydroxylated by the oxidizing enzyme encoded by the recombinant nucleic acid comprised in the host cell.

In further embodiments, methods are provided for producing diols, preferably α,ω-diols, which, in addition to the step described above, further comprise the step of recovering diols or α,ω-diols from the host cell or the culture medium. Suitable purification can be carried out by methods known to the person skilled in the art such as by using lysis methods, extraction, ion exchange, electrodialysis, ultrafiltration, nanofiltration, etc.

Yet a further aspect relates to diols, preferably α,ω-diols, obtainable by the methods disclosed herein. The present invention will now be further illustrated by means of the following non-limiting examples.

### EXAMPLES

### Example 1: Production of (Acetylated) (Symmetrical) α,ω-Bola Sophorosides Free from α,ω-1-Bola Sophorosides and (Acetylated) (Symmetrical) α,ω-Bola Glucosides Free from α,ω-1-Bola Glucosides

Materials and Methods

Strains, Media and Culture Conditions

As parental strain, the SL deficient Δcyp52M1 strain S. bombicola was used (Van Bogaert et al., 2013). A S. bombicola fatty alcohol oxidase fao1 knockout was obtained similarly as described by Van Renterghem et al., (2018) by integrating the ura3 gene under the regulatory control of its own promotor and tyrosine kinase (tk) terminator at the fao1 locus in the Δcyp52M1 strain. Three transformant colonies of the new strain were evaluated in terms of growth and glycolipid production, in parallel with the Δcyp52M1 parental strain. Cultivation, selection and transformation were performed as described by Lodens et al., (2018).

Production experiments using S. bombicola were performed using the production medium described by Lang et al., (2000). For shake flask experiments, 5 mL tube cultures were set up 24 h (30° C.), before transferring to shake flasks (4% inoculation). Production experiments were executed with feeding of fatty alcohols: oleyl alcohol, stearyl alcohol, lauryl alcohol, myristyl alcohol or cetyl alcohol which were added after 48 h of cultivation. The Δcyp52M1Δfao1 strain was also assessed without addition of the hydrophobic alcohol. For every production experiment, cultivation was stopped when glucose was depleted from the medium. Experiments were performed in duplicate, and average values with standard deviations are presented.

Molecular Techniques

General Techniques

General molecular techniques were employed as described by Green & Sambrook, (2012). Linear deletion cassettes were generated from vector backbones cloned and maintained in E. coli, based on the pGEM-T (Promega) and pJET (Thermo Fisher) vectors and cloning steps are described below. All primer sequences are represented in Table 1.

TABLE 1
Primers used for the creation of knockout strain
of the S. bombicola fatty alcohol oxidase gene
(fao1) in the Δcyp52M1 strain.

| Primer names | ′5-sequence-3′ |
|---|---|
| P30_FOR_checkpromIN | AAGGCGGGCTGGAATGCATATCTGAG (SEQ ID NO 1) |
| P34_REV_checkcassIN | GATGTCGAATAGCCGGGCTGCTAC (SEQ ID NO 2) |
| P276_pJET_Rev | AAGAACATCGATTTTCCATGGCAG (SEQ ID NO 3) |
| P625_pJET_For | CGACTCACTATAGGGAGAGCGGC (SEQ ID NO 4) |
| P1001_FOR_FAO1_ | CTCGAGTTTTTCAGCAAGATTGCCAAGTCGTTCAACACAG |
| extgibpJET | (SEQ ID NO 5) |
| P1002_REV_FAO1_ | AGGAGATCTTCTAGAAAGATCTGAGACAGCAGCTTGTCAC |
| extgibpJET | (SEQ ID NO 6) |
| P1003_FOR_ | GTGACAAGCTGCTGTCTCAGATCTTTCTAGAAGATCTCCTA |
| pJETextGib_FAO1 | C (SEQ ID NO 7) |
| P1004_REV_ | CTGTGTTGAACGACTTGGCAATCTTGCTGAAAAACTCGAGC |
| pJETextGib_FAO1 | CATC (SEQ ID NO 8) |
| P1020_FOR_FAO1 | TGCCAAGTCGTTCAACACAG (SEQ ID NO 9) |
| P1021_REV_FAO1 | CTGAGACAGCAGCTTGTCAC (SEQ ID NO 10) |
| P1022_REV_ | GCCTTGGCATTCAACATCTCAGGGAATC (SEQ ID NO 11) |
| checkFAO1KO | |
| P1023_FOR_ | GCACGCCCTTAGCTTCAGAG (SEQ ID NO 12) |
| checkFAO1KO | |
| P1062_FOR_Pura3_ | GACTGAGATGACGGAAGAGGCCCGAACATACCAGTTTCGC |
| extgib_upFAO1 | (SEQ ID NO 13) |
| P1063_REV_tTK_ | AAGCTTAGTGAGATCCGCGTGAACAAACGACCCAACACCC |
| extgib_downFAO1 | (SEQ ID NO 14) |
| P1064_FOR_downFAO1_ | GGGTGTTGGGTCGTTTGTTCACGCGGATCTCACTAAGCTTC |
| extgibtTK | (SEQ ID NO 15) |
| P1065_REV_upFAO1_ | GCGAAACTGGTATGTTCGGGCCTCTTCCGTCATCTCAGTC |
| extgibPura3 | (SEQ ID NO 16) |
| P1347_FOR_FAO1_ | GCCAGTGCAACAAGTATGAG (SEQ ID NO 17) |
| up_check | |
| P1348_REV_FAO1_ | GACCAGGCTAAACGCATCAC (SEQ ID NO 18) |
| down_check | |

Creation of the Δcyp52M1ΔFao1 Knockout Strain

The creation of the fao1 knockout cassette is described Van Renterghem et al., (2018) and was used to transform the S. bombicola Δura3::0Δcyp52M1::Pgapd_hph_Ttk strain, or further on called the Δura3Δcyp52M1 strain. The hph gene was isolated from Streptomyces hygroscopus, and encodes for hygromycin B phosphotransferase resistance (Gritz & Davies, 1983). After transformation, the ura3 positive colonies were selected on selective SD medium. Correct integration of the cassette was confirmed by colony PCR. For the newly-created Δura3::0 Δcyp52M1::Pgapd_hph_tTK Δfao1::Pura3_ura3_Ttk strain, further on called Δcyp52M1Δfao1, respectively, three successful colonies were chosen.

Downstream Processing and Characterization

The purification of the generated products of the Δcyp52M1Δfao1 strain when fed with oleyl was done by performing alkaline hydrolysis (pH 12, 5 M NaOH, 37° C., 1 h) to fully deacetylate the glycolipids to obtain a more uniform product for analysis. The purified and dried product was further purified by preparative liquid chromatography (PLC) for NMR analysis (see below).

Preparative Layer Chromatography (PLC)

Uniplate 20×20 cm PLC plates coated with 2 mm silica gel, impregnated with a green fluorescent indicator (F254) (Analtech) were used. First, 100 mg of sample was dissolved in MilliQ water. Subsequently, the solution was applied as a long streak at 2 cm from the bottom of the plate. The PLC plate was put in a solvent chamber containing the SL solvent mixture chloroform/methanol/water (65/15/2, v/v/v) (Asmer et al., 1988). After solvent development and respective evaporation, the plate was put under UV light at 254 nm. Subsequently, the highlighted zone of interest was scraped off using a scalpel and the scraped-off silica gel was collected. The compound was subsequently resolved by adding 20 mL MilliQ water to the falcon and centrifuged for 10 minutes at 4500 rpm. The supernatant was collected, and the process was repeated. The total supernatant, containing the bola SS product, was filtered (cut-off 0.22 μm, Millex® GV) to remove residual silica gel particles. Finally, the water was removed by using an Alpha 1-4 lyophilisator (Christ) to obtain a dry and highly-pure product, suitable for NMR analysis.

Analytical Techniques

Follow-Up of Growth

Optical density (OD) of cultures was measured at 600 nm using the Jasco V 630 Bio spectrophotometer (Jasco Europe) of 1 mL samples diluted with physiological solution (9 g/L NaCl). The viability of yeast cells in cultivation experiments was assessed by determining colony forming units (CFUs) which were expressed as the average logarithm of CFUs per culture volume as log(CFU/mL) (Saerens, Saey, et al., 2011). Alternatively, Cell Dry Weight (CDW) was determined by centrifuging 1 mL fermentation samples at 14000 rpm for 5 min in tared Eppendorf tubes, and subsequently washing the cell pellets twice with 1 mL physiological solution. The remaining cell pellet was put into a 70° C. oven for 5 days and then weighed afterwards. The CDW (g/L) was calculated after subtraction of the empty weight of the Eppendorf tube.

Follow-Up of Glucose Concentration

Glucose concentrations were determined using the 2700 Select Biochemistry Analyser (YSI Inc.) or using Ultra Performance Liquid Chromatography (Waters Acquity H-Class UPLC), coupled with an Evaporative Light Scattering Detector (Waters Acquity ELSD Detector) (UPLC-ELSD). For the UPLC analysis, an Acquity UPLC BEH Amide column (130 Å, 1.7 μm, 2.1×100 mm) (Waters) was used at 35° C. and an isocratic flow rate of 0.5 mL/min of 75% acetonitrile and 0.2% triethylamine (TEA) was applied (5 min/sample). For the ELS detection, the nebulizer was cooled to 15° C. and the drift tube was kept at a temperature of 50° C. The linear range was found to lie between 0 and 5 g/L glucose, using a gain of 100 for ELS detection (Empower software). To express glucose consumption, a linear curve was fitted through the obtained glucose concentrations by UPLC-ELSD, and the respective slope was taken and denoted as the glucose consumption rate (g/L·h).

Analysis of Glycolipids/Glycosides

Samples for glycolipid analysis were prepared by shaking a mixture of 1 mL of pure ethanol and 0.5 mL of fermentation broth vigorously for 5 minutes. Subsequently, after centrifugation for 5 minutes at 15000 rpm, the cell pellet was removed and the supernatant was filtered using a PTFE filter (cut-off 0.22 μm, Novolab) and adequately diluted in 50% ethanol (unless stated otherwise) before analyzing on (Ultra) High Pressure Liquid Chromatography-Mass Spectrometry ((U)HPLC-MS) and (U)HPLC-ELSD (Evaporative Light Scattering Detector).

HPLC-MS was performed using an LC (Shimadzu), coupled to an MS (Micromass Quattro LC) detection system. The different components were separated by polarity on a Chromolith Performance RP-18 Endcapped 100-4.6 mm column (Merck KGaA) at 30° C. The LC-MS method uses a gradient elution based on two solvents: MilliQ with 0.5% acetic acid, and pure acetonitrile (ACN). During the analysis, a flow rate of 1 mL/min was applied. The gradient starts with 5% acetonitrile and increases linearly until 95% over the course of 40 min. After this, the 95% acetonitrile is held for another 10 min, after which this is brought back to 5% acetonitrile in 5 min. The total analysis time per sample is 60 min/sample. The scanning range of the MS was set to 215-1100 g/mol. Using similar conditions as mentioned for HPLC-MS, HPLC-ELSD analysis was performed by Varian Prostar HPLC (ThermoScientific), coupled with an 2000ES ELSD (Alltech) at 40° C. All other conditions are similar as mentioned for the HPLC-MS.

UPLC-ELSD analysis was performed on a Acquity H-Class UPLC (Waters) and Acquity ELSD Detector (Waters), employing the same column and analysis method as UPLC-MS. For the ELSD detection, the nebulizer was cooled at 12° C. and the drift tube was kept at a temperature of 50° C., the gain was set to 200. To quantify the glycolipids, a dilution series of purified product was prepared. An available C18:1 acetylated bola SS purified batch (batch number SL24A) and purified acetylated C16:0 alkyl SS batch (batch number aAlkC16_2) were employed for quantification of bola SS and alkyl SS, respectively.

Alternatively, UPLC-MS was performed with an Accela (ThermoFisher Scientific) and Exactive Plus Orbitrap Mass Spectrometer (ThermoFisher Scientific). For glycolipid analysis, an Acquity UPLC CSH C18 column (130 Å, 1.7 μm, 2.1 mm×50 mm) (Waters) and a gradient elution system based on 0.5% acetic acid in milliQ (A) and 100% acetonitrile (B) at a flow rate of 0.6 mL/min was applied. The method was as follows: the initial concentration of 5% B (95% A) increases linearly until 95% B (5% A) during the first 6.8 min, and then linearly decreases again to 5% B (95% A) during 1.8 min. Subsequently, 5% B (95% A) is maintained until the end of the run (10 min/sample). Negative ion mode was used, and 2 μL samples were injected. MS detection occurred with a Heated Electrospray Ionization (HESI) source and conditions were set to detect masses ranging from 215-1300 m/z in a qualitative way.

All ¹H and ¹³C NMR spectra were recorded at 400 and 100.6 MHz, respectively, on a Bruker Avance III, equipped with ¹H/BB z-gradient probe (BBO, 5 mm). DMSO-[D6] was used as solvent, and as internal chemical shift standard (2.50 ppm for ¹H and 39.52 ppm for ¹³C). All spectra were processed using TOPSPIN 3.2 software. Attached Proton Test (APT), ¹³C, COSY, and HSQC spectra were acquired through the standard sequences available in the Bruker pulse program library. Custom settings were used for HMBC (32 scans), TOCSY (100 millisec MLEV spinlock, 0.1 sec mixing time, 1.27 sec relaxation delay, 16 scans), and H2BC (21.8 millisec mixing time, 1.5 sec relaxation delay, 16 scans), according to literature (Gheysen et al., 2008; Petersen et al., 2006).

Statistical Analysis

When two different groups were compared, the Welch's test was performed with a 95% confidence level using GraphPad Prism 7.04 software. For multiple group comparisons, analysis of variance (ANOVA) with Bonferroni's multiple comparison test correction with a 95% confidence level using GraphPad Prism 7.04 software was employed. For parameters pH, CFU, OD and glucose consumption, average values were taken when the yeast cells attained their stationary phase (after 48 h of cultivation). In general, data represented in graphs are the average and standard deviation of two experimental replicates (unless stated otherwise).

Results

Construction of Knockout Strains

The fatty alcohol oxidase fao1 knockout cassette (described in Van Renterghem et al., 2018)) was used to transform the S. bombicola Δcyp52M1Δura3 strain. After selection of the ura3⁺ colonies on selective SD plates, correct integration at both sides of the knockout cassette was controlled by performing colony PCR using two primer combinations (see Table 1). Three correct colonies of the newly created Δcyp52M1Δfao1 strain were selected for further characterization. The three selected transformants of the novel strain behaved similar to each other in terms of OD, CFUs, glucose consumption and glycolipid production. Therefore, only one colony is discussed in the next section for comparison with the parental Δcyp52M1 strain in terms of growth, pH, glucose consumption and glycolipid production.

Initial Characterization of Knockout Strain

For the wild type S. bombicola, colza oil (60-80% oleic acid) or pure oleic acid results in the best SL titers (Asmer et al., 1988; Rau et al., 2001) i.e. a hydrophobic substrate with C18 carbon chain length. Therefore, the newly created fool knockout strain was first assessed on shake flasks fed with a C18 fatty alcohol, i.e. oleyl alcohol (C18:1) as hydrophobic substrate.

Growth, pH and Glucose Consumption

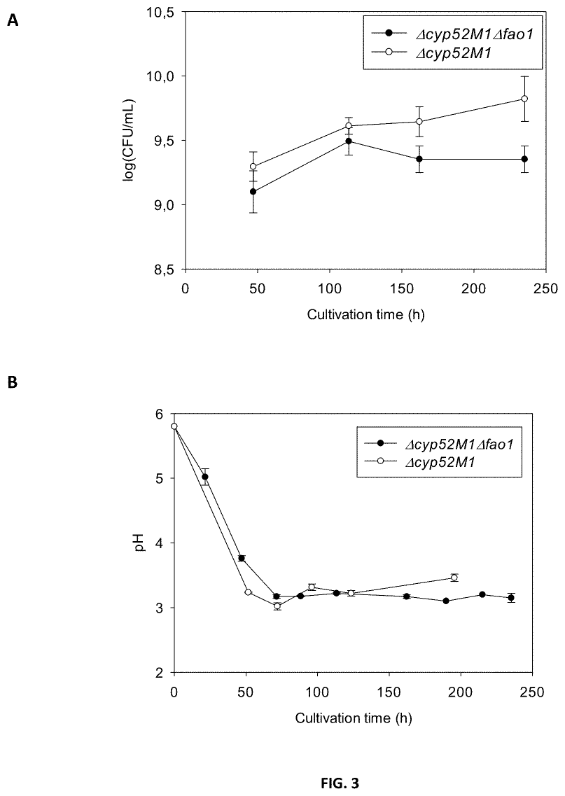

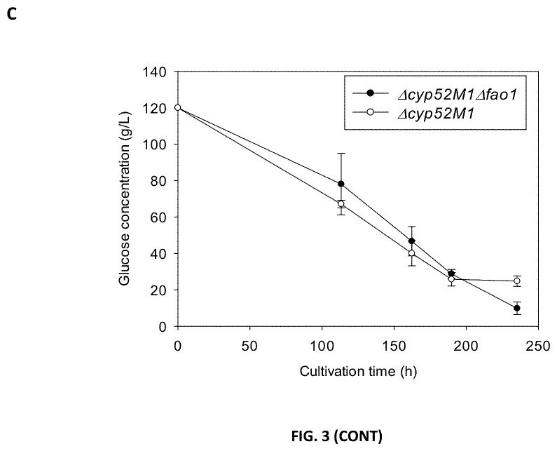

Important parameters such as log(CFU/mL), pH and glucose consumption are depicted in FIG. 3. Average values and standard deviations of assessed duplicates are presented. OD₆₀₀ evolution was not presented, due to interferences of the measurements with the oleyl alcohol substrate. In terms of log(CFU/mL) values in function of cultivation time (FIG. 3A), no significantly different CFU values were obtained between the new strain and reference strain. The observed pH drop was equal (FIG. 3B). The glucose consumption rate for the Δcyp52M1Δfao1 and parental strain are similar (0.47±0.06 and 0.47±0.01 g/L·h), as presented in FIG. 3C.

Glycolipid Production

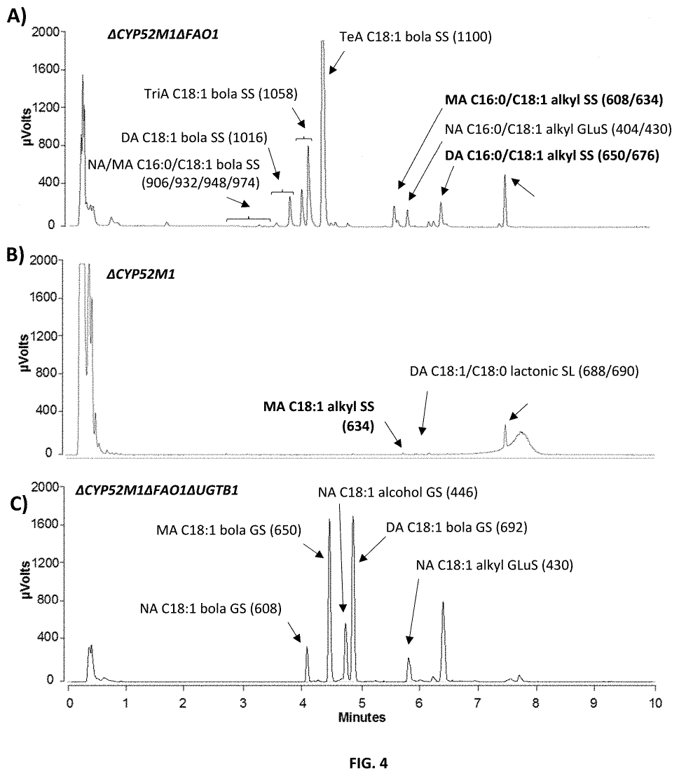

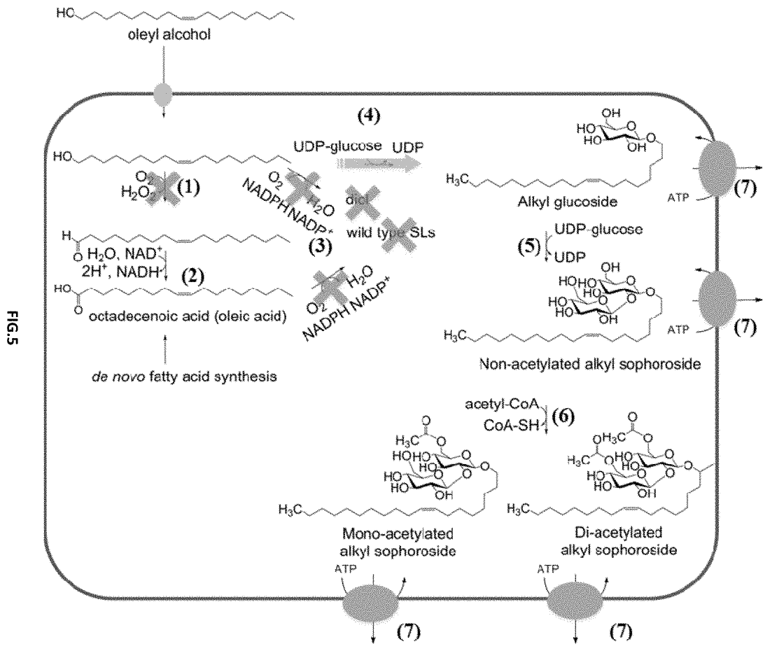

When looking at glycolipid production of the assessed strain, depicted in FIG. 4, distinctly different production profiles for the Δcyp52M1Δfao1 strain can be observed compared to the Δcyp52M1 parental strain.

The parental Δcyp52M1 strain (FIG. 4B) produced none (or very minor) amounts of glycolipids/glycosides (<7 min). A small peak corresponding to oleic acid (282 g/mol) is visible at later retention times (>7 min) (similarly as the one observed for the Δcyp52M1Δfao1 strain). The fact that almost no alkyl SS are detected, indicates that the fed oleyl alcohol is mainly oxidized to the corresponding oleic acid by the still functional ω-oxidation pathway (as FAO is not knocked out). Subsequently, as hydroxylation of oleic acid is inhibited by the cyp52M1 knockout, the glucosyltransferases cannot glycosylate the fatty acid, so the oleic acid accumulates or is broken down by the β-oxidation pathway.

In contrast to the parental strain, for the Δcyp52M1Δfao1 strain (FIG. 4A) glycoside production is clearly observed. Significant amounts of glycosides were found at retention times between 3.0-4.5 min and minor amounts between 5.5-6.5 min. After UPLC-MS analysis, surprisingly it was found that the masses in the early retention range (3-4.5 min) actually corresponded to bola SSs (900-1100 g/mol range), instead of the aimed for alkyl SS (550-700 g/mol range). Alkyl SS were indeed detected, but only in the abovementioned minor amounts between 5.5-6.5 min. This synthesis of bola SSs is theoretically (based on the art) impossible in this strain, as diol formation (see FIG. 5) (necessary for bola SS synthesis) through hydroxylation of the fed fatty alcohol should not be possible due to the deletion of the cyp52M1 gene. Indeed, no such action is observed in the parental strain (see FIG. 4B), which, as mentioned in the art indeed does not show any oxidation/hydroxylation activity towards oleyl alcohol (see FIG. 4B) nor on oleic acid and/or rapeseed oil (Van Bogaert et al., 2013).

Thus, surprisingly, the Δcyp52M1Δfao1 strain mainly produces bola SSs when fed with oleyl alcohol (FIG. 6), corresponding to the early retention times (3.0-4.5 min). As the AT gene is not knocked out in this strain (in contrast to the strain designed to produce bola SSs (Soetaert et al., 2013; Van Renterghem et al., 2018)) acetylation of the bola SSs is also observed for this new strain described above. The tetra-acetylated C18:1 bola SS (1100 g/mol) at 4.4 min is the most abundant component of the produced bola SSs, followed by the less abundant tri- and di-acetylated ones (1016 and 1058 g/mol, respectively). Non- and mono-acetylated C18:1 bola SS were also detected (respective 932 and 974 g/mol). The fact that the mono-, di- and tri-acetylated bola SS appear in two different peaks at 4.05 and 4.15 min, is probably explained by a different acetylation pattern of the molecule, similarly as shown for acidic SLs shown in literature (A. M. Davila et al., 1993; Saerens, 2012; Saerens, Saey, et al., 2011). Besides C18:1 bola SSs, also C16:0 based SSs (906 g/mol) are detected arising from the C16 amounts in the fed oleyl alcohol substrate (contaminating amounts of 2-10% cetyl alcohol or 1-hexadecanol are present in the substrate). Minor amounts of alkyl SSs were also produced and mainly C18:1 non-, mono- and di-acetylated alkyl SSs were detected (592/634/676 g/mol, respectively), but also traces of C16:0 alkyl SSs were found (566/608/650 g/mol, respectively).

It thus appears that the fed alcohol is favorably hydroxylated to the corresponding diol, and as such goes through the glycosylation cycle of UGTA1 and UGTB1 twice, to give rise to bola SSs (see FIG. 6), instead of just accumulating into alkyl SSs. As the parental strain fed with alcohols did not produce these compounds when fed on oleyl alcohol, it appears that the combination of the cyp52M1 and fao1 knockout is both crucial and unexpected.

To enable detailed characterization of the produced compounds, the bola SSs and alkyl SSs were purified from the mixture as described.

NMR Structure Analysis

NMR analysis was performed on a purified non-acetylated C18:1 bola SSs purified product derived from the broth of the Δcyp52M1Δfao1 strain.

Surprisingly, NMR analysis showed that in the produced bola SSs, both sophorose moieties are exclusively linked in a terminal (w) fashion to the C18:1 hydrophobic linker, presented in FIG. 7. A full symmetrical glycoside molecule is thus obtained, with chemical formula C₄₂H₇₆O₂₂. The term ‘symmetrical’ is used to indicate that the double bond is present in the middle of the compound.

Feeding Different Chain Lengths of Fatty Alcohols

Unexpectedly, no successful uniform production of alkyl SSs was obtained for the Δcyp52M1Δfao1 strain when feeding with oleyl alcohol. Instead, mixtures of bola SSs and alkyl SSs were found, although the majority of the novel glycosides were (acetylated) bola SSs and only minor amounts of alkyl SS were produced. The production of tetra-acetylated bola SS was unexpected, since the CYP52M1 enzyme was believed to be the only important hydroxylating/oxidizing enzyme involved in the first step of glycolipid production in S. bombicola. To assess the influence on the chain lengths of primary alcohols to the glycoside production, different substrates were fed to the Δcyp52M1Δfao1 strain. The parental strain Δcyp52M1 was assessed in parallel. Medium and long-chain alcohols were selected between a chain length of 12 and 18, similarly as reported (A.-M. Davila et al., 1994) for alkanes. Similarly as for the experiments described above, the hydrophobic substrate was added after 48 h of cultivation.

Growth, pH and Glucose Consumption

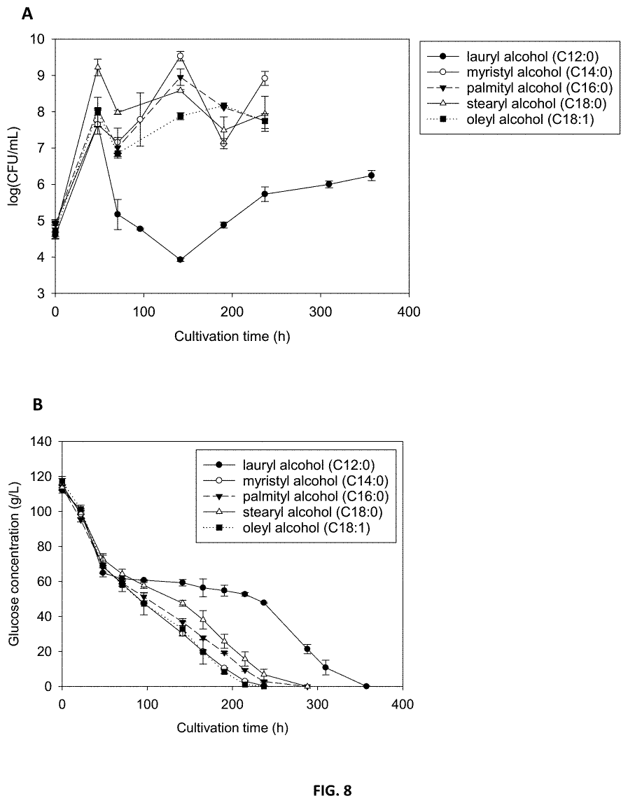

The addition of lauryl alcohol, myristyl alcohol, cetyl alcohol or oleyl alcohol did not significantly influence the CFU values for the Δcyp52M1 strain compared to the Δcyp52M1Δfao1 strain (FIG. 8). However, a significant drop in CFUs was noticeable when lauryl alcohol was added, in contrast to all other fed substrates (FIG. 8A). Significant higher glucose consumption rates were found when myristyl or oleyl alcohol were fed compared to cetyl or stearyl alcohol (0.48 g/L·h vs 0.38 g/L·h). After addition of lauryl alcohol (48 h after inoculation), glucose consumption stopped. Only for the Δcyp52M1Δfao1 strain, the glucose consumption initiated again (0.33 g/L·h) 100 h after substrate addition, until all glucose was depleted. This is in line with the CFUs evolution (FIG. 8A).

The Δcyp52M1Δfao1 strain was also studied when no hydrophobic substrate was added. The CFUs and glucose consumption were not significantly different from the cultures fed with alcohols (except lauryl alcohol).

Glycoside Production

Before addition of the primary alcohol, none of the Δcyp52M1Δfao1 and Δcyp52M1 cultures showed quantifiable glycolipid production. This was expected, as de novo fatty acids cannot be implemented into the glycolipid production pathway due to the cyp52M1 knockout, and the alcohol has to be fed in order to initiate novel glycoside production. The latter could be circumvented through further engineering which would allow the yeast strain to convert de novo synthetized fatty acids into fatty alcohols as described above. The expression of e.g. carboxylic acid reductase (Kalim Akhtara et al., 2013) enzymes converting fatty acids into fatty aldehydes, which can subsequently be converted into fatty alcohols e.g. by the simultaneous expression of an aldehyde reductase or an aldehyde decarboxylase (Fatma et al., 2016; Kang & Nielsen, 2017) would allow this.

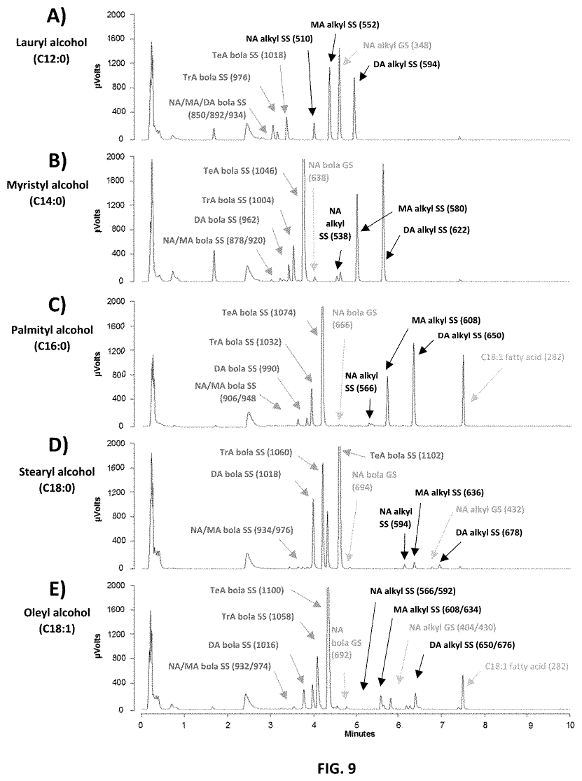

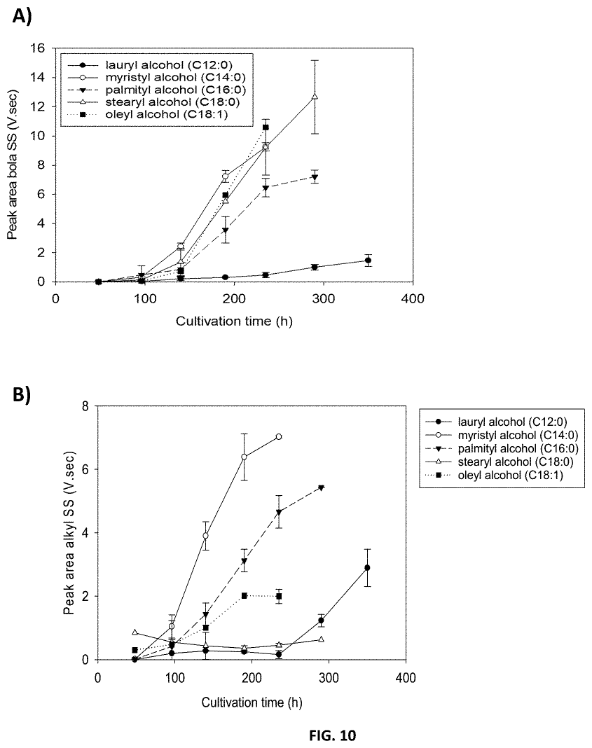

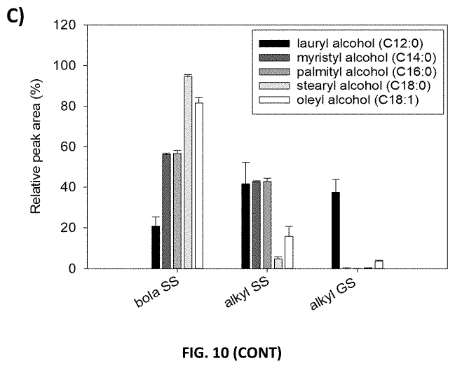

After addition of the hydrophobic substrate for the Δcyp52M1 strain, no peaks were visible after analysis on ELSD, which is in line with the expectations and with what has been described in the art (Van Bogaert et al., 2013). Glycoside end production profiles of the Δcyp52M1Δfao1 strain for the different fed primary alcohols analyzed on UPLC-ELSD are represented in FIG. 9 and their appearance throughout the experiments in FIGS. 10A and B and the respective ratio's in FIG. 10C. For each alcohol that was fed, a mixture of acetylated bola and alkyl SSs was produced. As expected, all produced glycosides originated from the fed alcohol directly. The latter is due to the fact that the cyp52M1 gene is knocked out, hence eliminating implementation of any de novo (mainly C16-C18) or generated (possible oxidation of the alcohol) fatty acids.

For lauryl alcohol, from non- up to tetra-acetylated C12:0 bola SS were detected (580/934/976/1018 g/mol). Besides C12:0 bola SS, also non- to di-acetylated C12:0 alkyl SS (510/552/594 g/mol) were detected at later retention times. However, the most abundant peak in the chromatogram corresponded to C12:0 alkyl glucosides (GLuS) (348 g/mol).

For myristyl and cetyl alcohol, very similar production profiles were observed. Clear retention times corresponding to C14:0 or C16:0 bola SSs (3.00-4.00 min and 3.00-4.3 min) and C14:0 or C16:0 alkyl SSs (4.5-5.8 min and 5.2-6.5 min) were distinguished with respective acetylations. C14:0 and C16:0 non-acetylated alkyl GLuSs (376 and 404 g/mol) were detected using MS. Minor quantities of di-acetylated C14:0/or C16:0 alcohol SS (or bola GL) (638 or 666 g/mol) were detected.

When the Δcyp52M1Δfao1 strain is fed with stearyl alcohol, again mixed production of acetylated C18:0 bola SSs (934/976/1018/1060/1102 g/mol) and C18:0 alkyl SS (594/636/678 g/mol) is observed. Additionally, minor amounts of C18:0 alkyl GLuSs (432 g/mol) were detected. Similarly as for myristyl and cetyl alcohol, di-acetylated C18:0 alcohol SSs (or bola GSs) (694 g/mol) were detected.

Addition of oleyl alcohol resulted in the glycolipid production profile described above. Also non-, mono- and di-acetylated C16:0 and C18:1 alkyl SS (566/608/650 g/mol and 592/634/676 g/mol respectively) and non-acetylated C16:0 and C18:1 alkyl GLuSs (404 and 430 g/mol) were detected. Similarly as for the other alcohols, di-acetylated C18:1 alcohol SS (or bola GL) (692 g/mol) was also detected.

Generating the triple knock out strain (ΔCYP52M1 ΔFAO1 ΔUGTB1) for the production of symmetrical bola glucosides.

For the generation of the triple knock out strain (ΔCYP52M1 ΔFAO1 ΔUGTB1), the ura3 marker was first removed from the double knock out strain (ΔCYP52M1 ΔFAO1) as described by Lodens et al., (2018). A linear UGTB1 knock-out cassette with ura3 marker was generated from plasmid “pGKO ugtB1” described by Saerens, Zhang, et al., (2011) using PfuUltra High Fidelity PCR (Stratagene) and the primer pair GTII-472F and GTII+239R (GTII-472For: 5′-GAGAGTGGGACCTGATTC-3′ (SEQ ID N° 19)/GTII+239Rev: 5′-CTGCTCTCAACACCGAGTGTAG-3′ (SEQ ID No 20)). This deletion cassette was transformed into the ura3 negative ΔCYP52M1 ΔFAO1 strain and correct transformants were selected.

The resulting strain produced (acetylated) symmetrical α,ω-bola glucosides as schematically shown in FIG. 11.

Three colonies were chosen and evaluated in terms of growth and glycolipid production and compared with the Δcyp52M1Δfao1 parental strain. The production experiment was performed with feeding of oleyl alcohol as the hydrophobic substrate. Culture conditions and analytical techniques were as mentioned above. The values for CDW, pH and glucose consumption and their production profiles were very similar for the three selected colonies. The pH drop was nearly identical for the Δcyp52M1Δfao Δugtb1 strain and the Δcyp52M1Δfao1 strain.

An UPLC-ELSD chromatogram of one of the assessed colonies of the Δcyp52M1Δfao1ΔugtB1 strain is shown in FIG. 4C together with the glycoside masses detected using UPLC-MS analysis. The mono- and di-acetylated C18:1 bola glucosides (GLuSs) were the most abundant compounds (650 g/mol at 4.45 min and 692 g/mol at 4.85 min respectively). The non-acetylated C18:1 bola GLuSs (608 g/mol) eluted at 4.05 min. Other glycosides (intermediates of bola GLuSs) were also detected: non-acetylated C18:1 alcohol GLuSs (446 g/mol at 4.72 min) and non-acetylated C18:1 alkyl GLuSs (430 g/mol at 6.38 min).

The production pathway of the alcohol- and bola glucosides based on oleyl alcohol is illustrated in FIG. 11. The alkyl glucosides were produced by direct glucosylation of the oleyl alcohol substrate at the hydroxyl group, similar as depicted in FIG. 6 for the alkyl sophorosides. The feeding of fatty alcohols with other chain lengths gave rise to bola GluSs with other chain lengths associated with the chain length of the fed substrate(s) similar as described above for bola SSs.

### Example 2: Production of ω (Acetylated) C9:0 ω-Sophoroside Aldehydes and C9:0 ω-Sophorolipids

Materials

Chemicals and Precursors

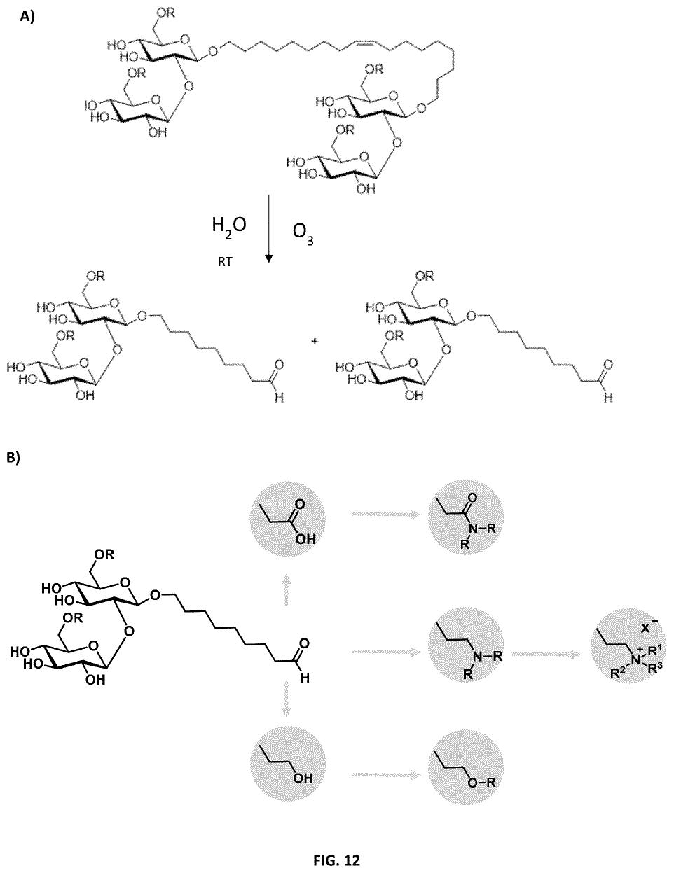

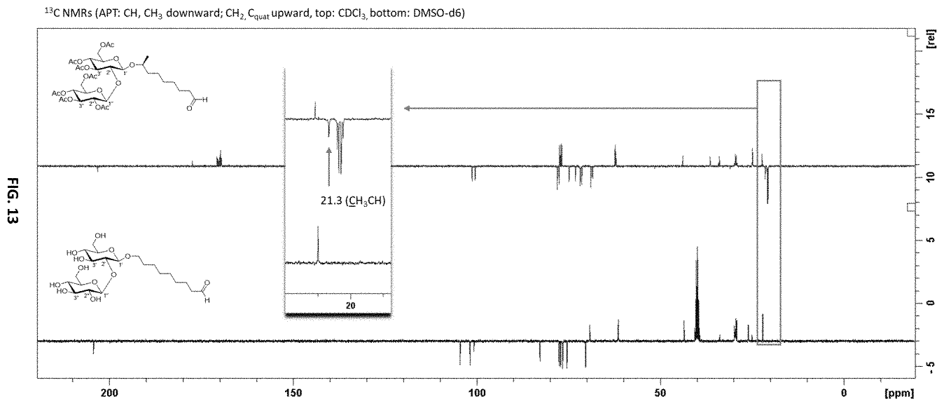

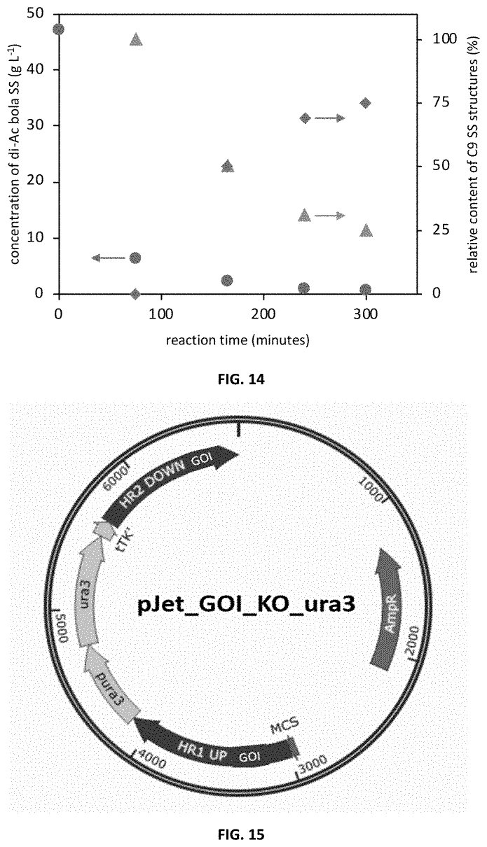

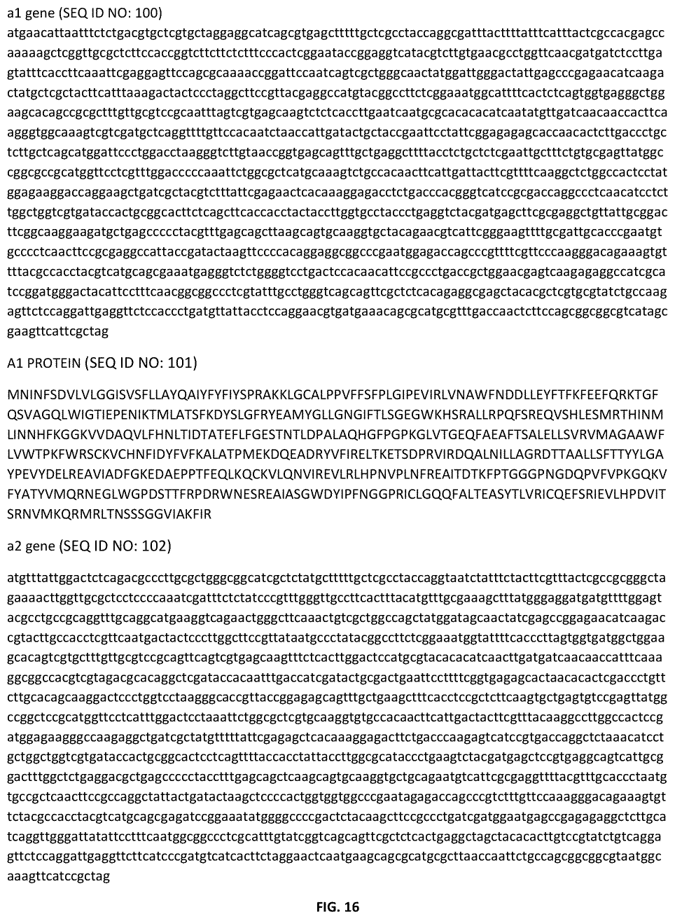

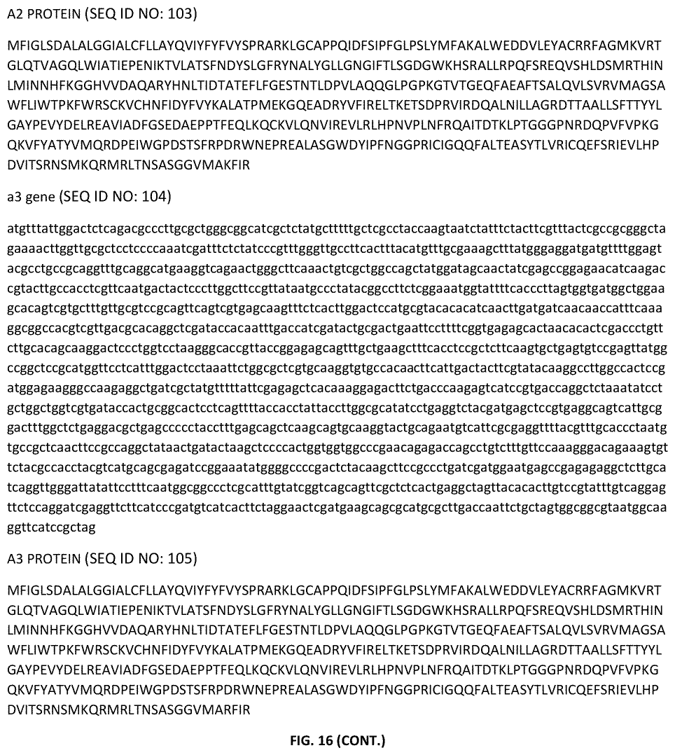

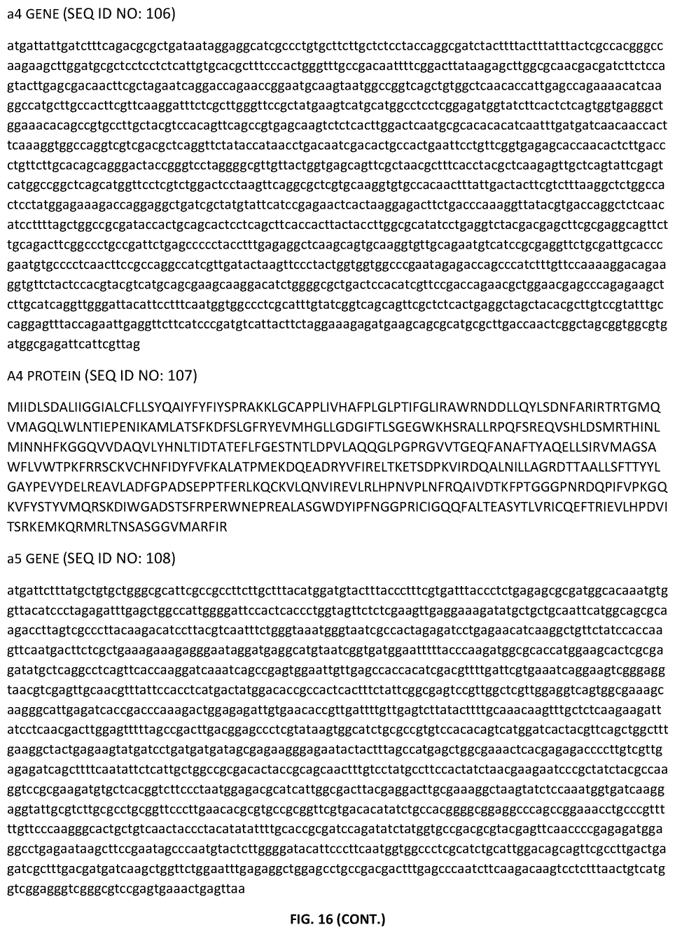

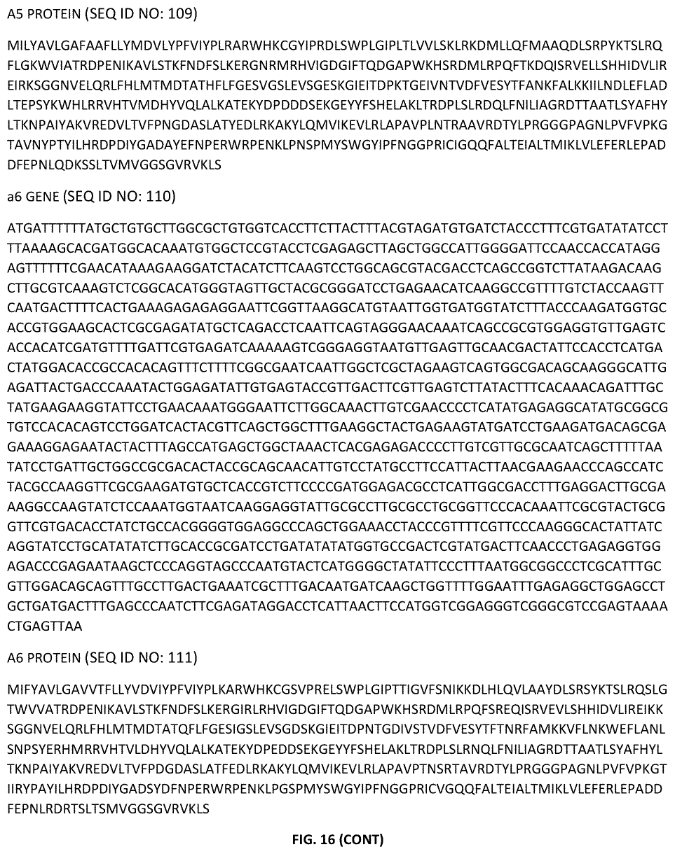

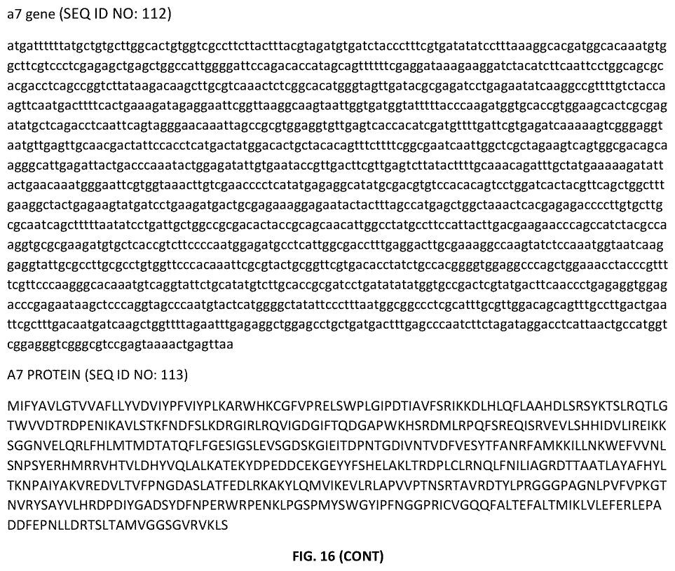

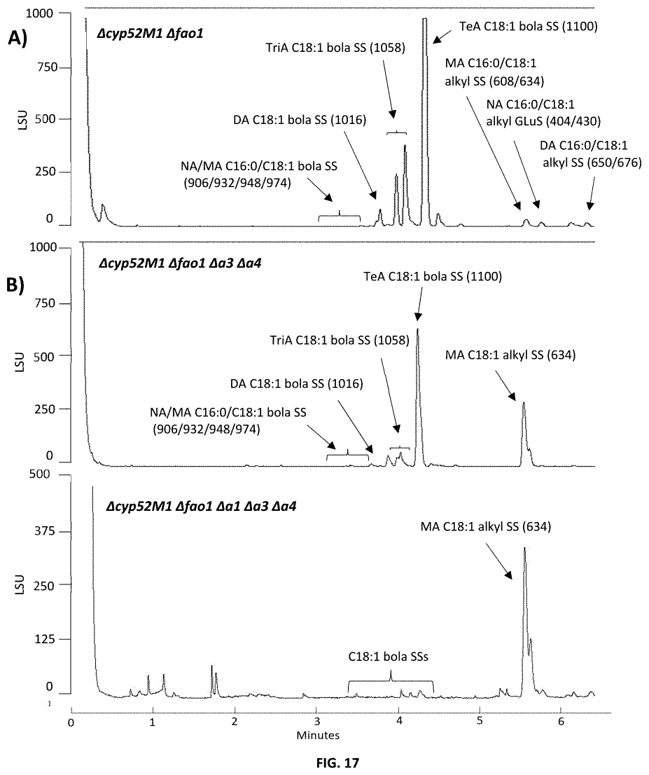

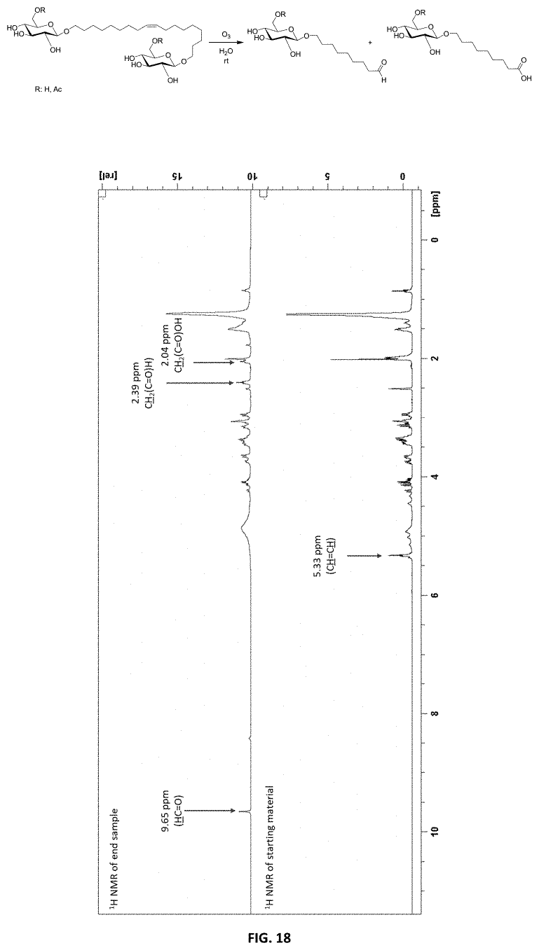

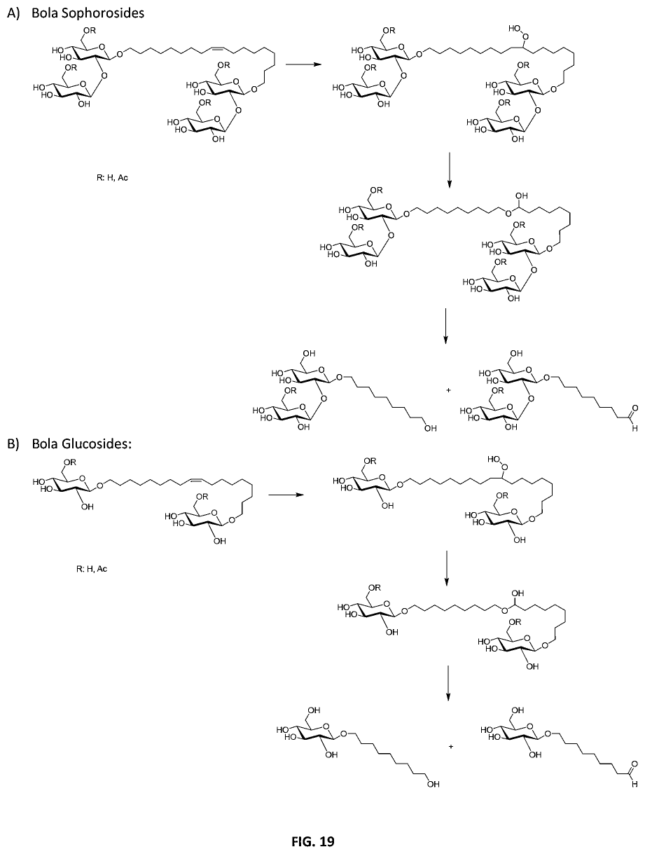

All chemical reagents (DMSO-d₆, MeOD-d₄, CDCl₃) were purchased from Sigma Aldrich and were used without further purification. Oxygen (99.9%, Air Liquide) was used as the starting material for the generation of ozone. The acetylated and non-acetylated C18:1 symmetrical α,ω-bola sophorosides (C18:1) (FIG. 12) were produced as described in example 1 and as described before (Van Renterghem et al., 2018).

Synthetic Procedures

Production of C9:0 ω-Sophoroside Aldehydes Via Ozonolysis at Laboratory Scale

The reaction was carried out in a laboratory type gas-washing bottle equipped with a fritted disc and a volumetric capacity of 300 ml. The bottle was connected to the ozone generator (Ozonia Triogen Model LAB2B). The flow of the O₃/O₂ mixture was measured using a mass-flow meter (Bronkhorst Flow-Bus E-7000). The concentration of the ozone in the off-gas stream was monitored with an ozone analyzer (Anseros Ozomat GM Non-Dispersive UV-analyzer). In a typical experiment, 10 g of acetylated (10.72 mmol) or non-acetylated (9.09 mmol) symmetrical C18:1 α,ω-bola sophoroside, dissolved in demineralized water (100 mL), was placed in the gas-washing bottle. The solution was mixed by a magnetic stirrer at a rate of 600 rpm. Ozone was produced by passing dry oxygen (99.9%, Air Liquide) as the feed gas through an ozone generator. The ozone was introduced to the reaction medium as finely dispersed gas bubbles via a glass frit located at the bottom of the gas-washing bottle. The total reaction time was calculated/estimated following the formula below.

$$t = \frac{n \times M {W}_{{O}_{3}}}{Q \times {c}_{{O}_{3}}}$$

Where:

t=reaction time (min),

n=moles of the double bond (mmol),

MW_{O}_{ₛ}=molecular weight of ozone (mg mmol⁻¹),

cₒ_{ₛ}=the concentration of ozone in the gas stream (mg l⁻¹)

Q=flow rate of the gas stream (L min⁻¹).

The reaction was followed by monitoring the off-gas ozone concentration with an ozone analyzer. As the reaction proceeded, the off-gas ozone concentration increased, eventually to the same concentration as the inlet gas, suggesting that the substrate has been completely converted. After reaching this point in the reaction, the feed gas was continued to be fed ⅕ more of the calculated reaction time to ensure the complete conversion of the starting material. At the end of the reaction, the obtained reaction mixture is freeze-dried at 0.05 mbar, yielding an off-white powder.

Production of C9:0 ω-Sophoroside Aldehydes Via Ozonolysis at Small Pilot Scale

The ozonolysis reaction at pilot scale was carried out in a stainless-steel fermenter with a 7 L reactor volume. The reactor was equipped with a temperature sensor, pH probe, air sparger to introduce the ozone/oxygen gas mixture and a 0.08 m diameter impeller able to operate between 100 to 1000 rpm. The reactor was placed in a Lexan cabinet in which two exhausters were installed to continuously remove the off-gasses. A Midas ozone detection sensor was installed to detect any possible gas leak. The ozonolysis reaction was performed using an Anseros COM-Ad-08 ozone generator (flow rate range: 0-300 L h⁻¹ and ozone concentration: max. 40 g h⁻¹ at 300 L h⁻¹). Oxygen gas was used to generate ozone. A mass flow meter was used to control the oxygen/ozone gas flow. The (acetylated) symmetrical C18:1 α,ω-bola sophoroside product was dissolved in RO water and transferred to the reactor. The progress of the reaction was followed by off-line HPLC-ELSD analysis. When the bola sophoroside concentration was reduced to zero, the ozone generation was stopped, and the solution was flushed with O₂ for a few minutes and then with N₂ for 30 minutes to remove any residual ozone. At the end, the reaction solution was collected through the outlet port into a gas bottle and kept at 4° C. As a final step, the reaction solution was freeze-dried by using a Virtis Genesis Pilot Lyophilizer.

Analytical Procedures

NMR Spectroscopy

¹H-NMR (400 MHz) and ¹³C-NMR (100 MHz) spectra were recorded on a Bruker Avance III Nanobay 400 MHz NMR spectrometer at 25° C. ¹H and ¹³C chemical shifts are reported in ppm and referenced to the residual solvent peak. The compounds were dissolved in deuterated solvents (DMSO-d₆ or MeOD-d₄). The assignment of the different peaks was performed using 2D-HSQC and 2D-COSY spectra.

Sample Treatment for HPLC-ELSD, UPLC-ELSD and UPLC-MS Analysis

Samples for glycoside analyses were prepared by shaking a mixture of 1 mL of pure ethanol and 0.5 mL of fermentation broth vigorously for 5 minutes. Subsequently, after centrifugation for 5 minutes at 15000 rpm, the cell pellet was removed and the supernatant was filtered using a PTFE filter (cut-off 0.22 μm, Novolab) and adequately diluted in 50% ethanol (unless stated otherwise) before analyzing on (Ultra) High Pressure Liquid Chromatography-Mass Spectrometry ((U)HPLC-MS) and (U)HPLC-ELSD (Evaporative Light Scattering Detector).

HPLC-ELSD Chromatography

UPLC-ELSD analysis was performed on an Acquity H-Class UPLC (Waters) and Acquity ELSD Detector (Waters), employing the same column and analysis method as UPLC-MS. For the ELSD detection, the nebulizer was cooled at 12° C. and the drift tube was kept at a temperature of 50° C., the gain was set to 200. To quantify the glycolipids, a dilution series of purified product was prepared. A purified acetylated symmetrical C18:1 α,ω-bola sophoroside batch (batch number SL24A) was employed for quantification of bola SSs.

LC-MS Chromatography

An Agilent apparatus with a Supelco Ascentis Express C18 column (L 3 cm×ID 4.6 mm) with 2.7 μm fused-core particles (90 Å pore size) was used for LC-MS analyses. This apparatus equipped with a UV detector (operating at 220.8 nm, 254.8 nm and 280.8 nm) and connected to an Agilent 1100 series LC/MSD type mass spectrometer with Electron Spray Ionization geometry (ESI 70 eV) and using a Mass Selective Detector (single quadrupole).

Alternatively, UPLC-MS was performed with an Accela (ThermoFisher Scientific) and Exactive Plus Orbitrap Mass Spectrometer (ThermoFisher Scientific). For glycolipid analysis, an Acquity UPLC CSH C18 column (130 Å, 1.7 μm, 2.1 mm×50 mm) (Waters) and a gradient elution system based on 0.5% acetic acid in milliQ (A) and 100% acetonitrile (B) at a flow rate of 0.6 mL/min was applied. The method was as follows: the initial concentration of 5% B (95% A) increases linearly until 95% B (5% A) during the first 6.8 min, and then linearly decreases again to 5% B (95% A) during 1.8 min. Subsequently, 5% B (95% A) is maintained until the end of the run (10 min/sample). Negative ion mode was used, and 2 μL samples were injected. MS detection occurred with a Heated Electrospray Ionization (HESI) source and conditions were set to detect masses ranging from 215-1300 m/z in a qualitative way.

Results

Production of C18:1 α,ω-Bola Sophorosides.

Acetylated and non-acetylated symmetrical α,ω-bola sophorosides C18:1 were prepared using the S. bombicola ΔCYP52M1 ΔFAO1 strain as described above and following the fermentation and purification methodology reported previously (Van Renterghem et al., 2018). The produced acetylated and non-acetylated symmetrical α,ω-bola sophorosides were subsequently used as starting/raw material/feedstock to produce C9:0 ω-sophorosides (SS) by cleavage of the double bond via ozonolysis (FIG. 12). The ozonolysis reaction was carried out in water to preserve the green character of this method. Moreover, ozonolysis in the presence of water offers a safer reaction environment and a means for the direct synthesis of aldehydes by limiting the production of unstable ozonide structures. The bola sophorosides are well water soluble in contrast to the lactone sophorolipids, which simplifies this method.

Ozonolysis of Symmetrical C18:1 α,ω-Bola Sophorosides.

Ozonolysis experiments were performed at lab- and pilot scale. An overview of the experimental parameters applied at lab-scale is given in Table 2. For both substrates, experiments were performed in triplicate.

TABLE 2
An overview of experimental parameters used
at lab-scale ozonolysis experiments.

| | acetylated | Non-acetylated |
|---|---|---|
| parameters | bola SS | bola SS |
| concentration of substrate | 100 | 100 |
| (aq.) (g L⁻¹) | | |
| volume of substrate solution | 0.10 | 0.10 |
| (aq.) (L) | | |
| pHt₀ | 4.5 | 9.5 |
| O₂/O₃ flow rate (L min⁻¹) | 0.14 | 0.14 |
| O₃ delivery (mg min⁻¹) | 4.32 | 4.32 |
| reaction time (min) | 122 | 144 |
| temperature (° C.) | 25 | 25 |
| mixing (rpm) | 600 | 600 |

Since the reaction mixture consists of sophorosides, which are biosurfactants, foaming was identified as an operational issue that could cause the entrainment of substrates out of the reactor during ozonolysis experiments. Therefore, an optimal substrate concentration of 100 g L⁻¹ for both substrates was selected based on preliminary tests where the foaming was limited. During the experiments, the foam stability of the non-Ac bola SS was higher (i.e. more persistent) compared to the Ac bola SS.

The solution of the non-acetylated α,ω-bola sophorosides (non-Ac bola SSs) gelled even at low concentrations of 10 g L⁻¹ making it a rather difficult substrate to work with. Therefore, proper mixing of the reaction mixtures was ensured throughout all experiments. Once the feeding of ozone was started, the gelling disappeared gradually due to the conversion of the non-Ac bola SS to C9:0 ω-sophoroside aldehydes. The slight difference in reaction time for both substrates is related with the difference in their molecular weights (see Experimental Section for the calculation of the reaction time) as both set of experiments were performed with an identical flow rate of O₃.

Although ozonolysis is typically performed at low temperature conditions due to the exothermic nature of ozonide decomposition, no significant increase in temperature was observed during the experiments. This observation suggests that the carbonyl oxide intermediate may have been effectively trapped by water, limiting the formation of ozonide.

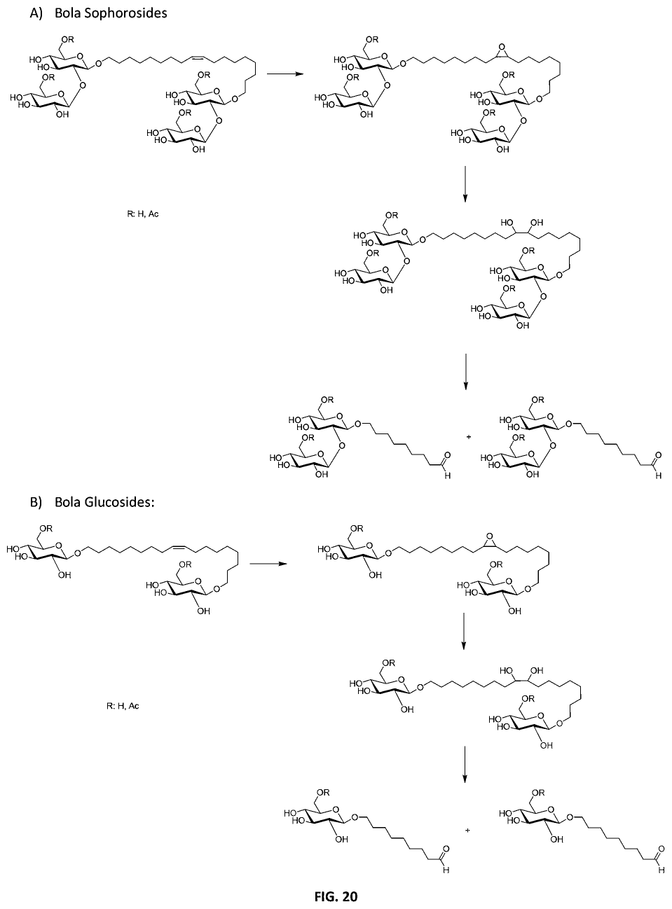

An NMR spectrum of the result is shown in FIG. 13 and compared to an NMR spectrum obtained for performing ozonolysis on wild type SLs as described by (Delbeke, Roelants, et al., 2016) The doublet peak at 21.3 ppm in the top NMR spectrum corresponds to the CH₃ group at the subterminal position (21.3 ppm, CH₃CH). There is not any peak at this chemical shift in the spectrum below. Thus only C9:0 ω-sophoroside aldehydes are formed after ozonolysis of the α,ω-bola sophorosides described in this invention.

The results of the experiments performed at lab-scale are presented in Table 3. The repeatability of the ozonolysis experiments was good. The major identified product for both substrates was the C9:0 ω-sophoroside aldehyde. Another product present in smaller quantities was identified as the C9:0 ω-sophorolipid carboxylic acid (C9:0 ω-sophorolipid). The relative contents of C9:0 ω-sophoroside aldehyde and C9:0 ω-sophorolipid was similar for both substrates. It seems that the presence or the absence of acetyl groups on the sophorose units had no notable effect on the outcome of the ozonolysis. Eliminating the need to perform reductive or oxidative workup to obtain the aldehyde and the acid products can be considered as an advantage of the employed reaction system. Using H₂O as the (co)-solvent was reported to give a similar result, which was attributed to the trapping of the carbonyl oxide intermediate by water (Schiaffo & Dussault, 2008).

TABLE 3
Relative contents of C9:0 ω-sophoroside aldehyde
and C9:0 ω-sophorolipid in the reaction mixture,
after applying ozonolysis conditions on (acetylated: R =
Ac) C18:1 α,ω-bola sophorosides, as determined by ¹H NMR.

| entry | substrate | C9:0 ω-sophoroside aldehyde | C9:0 ω-sophorolipid |
|---|---|---|---|
| 1 | R = Ac | 89.7 (±3.2) | 10.3 (±3.2) |
| 2 | R = H | 92.3 (±0.3) | 7.7 (±0.3) |

Following the initial set of experiments performed at lab-scale, ozonolysis experiments using Ac bola SS and non-Ac bola SS were conducted at pilot scale in a dedicated ozonolysis set-up. An overview of the experimental conditions employed during pilot-scale experiments are presented in Table 4. Although no temperature increase was observed during lab-scale experiments, necessary precautions were still taken at pilot scale as higher amounts of ozone and substrates were used. For instance, the concentration of substrates was limited to 50 g L⁻¹ to be able to contain the effects of any possible ozonide decomposition.

TABLE 4
An overview of experimental parameters used
at pilot-scale ozonolysis experiments.

| | Acetylated | Non-acetylated |
|---|---|---|
| parameters | bola SS | bola SS |
| concentration of substrate | 50 | 25 |
| (aq.) (g L⁻¹) | | |
| volume of substrate solution | 2.00 | 2.00 |
| (aq.) (L) | | |
| pHt₀ | 4.5 | 12 |
| O₂/O₃ flow rate (L min⁻¹) | 0.56 | 0.20 |
| O₃ delivery (mg min⁻¹) | 39.20 | 3.00-8.00 |
| reaction time (min) | 300 | 153 |
| temperature (° C.) | 15 | 22 |
| mixing (rpm) | 200 | 250 |

The concentration profile of Acetylated bola SS together with the evolution of the relative contents of C9:0 ω-sophoroside aldehyde and C9:0 ω-sophorolipid compounds are depicted in FIG. 14. The amount of Acetylated bola SS (monitored by off-line HPLC-ELSD analysis) decreased as the reaction proceeded and almost finished after 240 minutes. However, the evolution of the relative contents of C9:0 ω-sophoroside aldehyde and C9:0 ω-sophorolipid indicated that overoxidation occurred to an extent that the relative ratio of the C9:0 ω-sophorolipid to C9:0 ω-sophoroside aldehyde was 75:25 after 300 minutes.

Next, ozonolysis of non-Ac bola SS was also performed at pilot scale. The concentration of the solution (25 g L⁻¹) was lower compared to that of Ac bola SS (50 g L⁻¹) due to the gelling tendency of non-Ac bola SS at high concentrations. Upon the introduction of O₂/O₃ to the reaction mixture, significant foaming occurred. Therefore, a lower O₂/O₃ flow rate (i.e. a lower ozone concentration) was applied during non-Ac bola SS ozonolysis experiments. Increasing the reactor headspace pressure (5 bar) with N₂ helped to reduce the foaming. But the reaction was stopped after 153 min as the pH (from 12.0 to 6.5) dropped significantly hinting at the formation of C9:0 ω-sophorolipid. Despite the lower ozone concentration applied and the higher initial pH, the C9:0 ω-sophorolipid still formed. At the end of the reaction, the relative content of C9:0 ω-sophoroside aldehyde to C9:0 ω-sophorolipid was 65 to 35, as determined by ¹H NMR analysis. C9:0 ω-sophoroside aldehyde was obtained as an off-white powder, whereas C9:0 ω-sophoroside aldehyde/C9:0 ω-sophorolipid mixture was obtained as an off-white gel after freeze-drying.

The use of the (acetylated) symmetrical C18:1 α,ω bola sophorosides to generate two C9:0 ω-sophoroside aldehyde units per bola molecule by breaking the double bond through ozonolysis is an interesting route avoiding the loss of carbon described for wild type SLs and mixture of ω- and ω-1 compounds. Moreover, the high water solubility of these innovative compounds allows the use of water as a solvent without having to adapt pH etc. as is described in the art for wild type SLs (D. Develter & Fleurackers, 2008). Moreover, the use of water as the reaction solvent would have a second advantage as compared to the art i.e. no additional workup is required to derivatize to C9:0 ω-sophoroside aldehyde or C9:0 ω-sophorolipid, which is a serious advantage. Consequently, the ozonolysis of (acetylated) symmetrical C18:1 α,ω-bola sophorosides is an attractive option to produce C9:0 ω-sophorosides free of contaminating C9:0 ω-1 sophorosides.

### Example 3: Selective Production of Alkyl Sophorosides

Materials and Methods

Strains, Media and Culture Conditions

Escherichia coli Top10 strain was used for storing and replicating plasmids. E. coli strains were grown on Lysogeny Broth (LB) medium (10 g/L Tryptone, 5 g/L Yeast extract, 5 g/L NaCl (Brenntach), 15 g/L Agar) at 37° C., 200 rpm. Plasmid transformed E. coli strains were selected on LB medium supplemented with 50 mg/mL ampicillin.

Starmerella bombicola ATCC 22214 was used as the wild type (WT) strain and the thereof derived Δcyp52M1 Δfao1 strain described under example 1 was used as a parental strain. For the growth of S. bombicola, Yeast extract Peptone Dextrose (YPD) medium was used (20 g/L Glucose·H₂O, 10 g/L Yeast extract, 20 g/L Bacto peptone, 20 g/L Agar). The 3C-agar solid medium (110 g/L Glucose·H₂O, Yeast Extract 10 g/L, Urea 1 g/L, Agar 20 g/L) was used to count Colony Forming Units (CFU). The selective medium used depends: in order to select colonies that lost the ura3 marker, a medium containing 5-fluoroorotic acid (FOA), while a selective minimal medium without uracil was used to select for ura3 positive colonies (Van Bogaert et al. (2007)).

Production experiments using S. bombicola were performed using the production medium described by Lang et al., (2000). For shake flask experiments, 5 mL tube cultures were set up 24 h (30° C.), before transferring to shake flasks (4% inoculation). Production experiments were executed with feeding of hydrophobic substrates: oleic acid, oleyl alcohol, stearyl alcohol, lauryl alcohol, myristyl alcohol or cetyl alcohol which were added after 48 h of cultivation. Cultivation was stopped when glucose was depleted from the medium.

Molecular Techniques

General molecular techniques as described by Sambrook & Russell (2001) were applied. Genomic DNA (gDNA) extraction from S. bombicola was performed as described by Roelants et al. (2013) Circular Polymerase Extension Cloning (CPEC) was used in order to create circular plasmids from linear DNA fragments (insert piece and back-bone). In order to make knock-out strains, S. bombicola was transformed by electroporation with linearized DNA as described by Lodens et al. (2018) and plated onto selective medium and incubated at 30° C. until colonies appeared. When a novel S. bombicola knock out strain was generated, cryovials were generated and in a next step, the introduced ura3 marker was removed again by transforming the novel strain with a ura3 PT recovery cassette consisting of the ura3 promotor (P) fused to the ura3 terminator (T) and selecting for ura3 negative strains on a 5-FOA (5-fluoro-orotic acid) containing selection medium, toxic for ura3 positive strains, as described by Van Bogaert et al. (2007).

Correct integration of the knock out cassettes was confirmed by colony PCR.

Primers and their sequences used for the generation and check of a strain wherein the a1 gene (SEQ ID NO: 100) is deleted:

oCARBO9757:
(SEQ ID NO: 21)
ATTGTCCTCCAAGTAACTCGCGGTGGCAGTATAGTGAGTC
GTATTACAATTCACTGGC
oCARBO9758:
(SEQ ID NO: 22)
CGGCCAGTGAATTGTAATACGACTCACTATACTGCCACCG
CGAGTTACTTGGAGGAC
oCARBO9759:
(SEQ ID NO: 23)
CGAACACTGCCATCATGGTTCAACCTCACGAGGCCGTATT
GAACAGTTTGGACACTCTTG
oCARBO9760:
(SEQ ID NO: 24)
CAAGAGTGTCCAAACTGTTCAATACGGCCTCGTGAGGTTG
AACCATGATGGCAGTGTTCG
oCARBO9761:
(SEQ ID NO: 25)
ACTCATGATATGTATGCTCGAAGCACAGAACCGGGTATAC
TAGTGATTTGAACAAACG
oCARBO9762:
(SEQ ID NO: 26)
GGTCGTTTGTTCAAATCACTAGTATACCCGGTTCTGTGCT
TCGAGCATACATATCATGAG
oCARBO9763:
(SEQ ID NO: 27)
CTCCCATATGGTCGACCTGCAGGCGGCCAGTGCGCCGATC
TCACGCCGTGCTCATGC
oCARBO9764:
(SEQ ID NO: 28)
AGCATGAGCACGGCGTGAGATCGGCGCACTGGCCGCCTGC
AGGTCGACCATATGGGAGAG
oCARBO9958:
(SEQ ID NO: 29)
CTGCCACCGCGAGTTACTTGGAGGACAATA
oCARBO10025:
(SEQ ID NO: 31)
GGCATCATCATGACCACAAGAAGGAGGAGAAC
oCARBO10026:
(SEQ ID NO: 32)
GAAAGTCGGGCGAGTCCGTTTCTTCTCAGC

Primers and their sequences used for the generation and check of a strain wherein the a2 gene (SEQ ID NO: 102) is deleted.

oCARBO10076:
(SEQ ID NO: 32)
TCAGAAGCGTATTTCATCAGTTATCGGCCGCCTGCAGGTC
GACCATATG
oCARBO10077:
(SEQ ID NO: 33)
ATCTATGCTCAGACAGGGCTGACGTATTATGAAATCCCGC
GGCCATGGC
oCARBO10078:
(SEQ ID NO: 34)
CGGCCGCCATGGCCGCGGGATTTCATAATACGTCAGCCCT
GTCTGAGCATAG
oCARBO10079:
(SEQ ID NO: 35)
CAGTGTACTTGATTGATCAGAATTCGAACACTAGACAATG
TTTCAAAGGTTCTTAAG
(SEQ ID NO: 36)
oCARBO10080:
GCCTTAAGAACCTTTGAAACATTGTCTAGTGTTCGAATTC
TGATCAATG
(SEQ ID NO: 37)
oCARBO10081:
(SEQ ID NO: 38)
CTCATGGCGTGGCTTATGCGTAAAATAACCGGAAACAAAC
GACCCAACACC
oCARBO10082:
(SEQ ID NO: 39)
CGCACGGGTGTTGGGTCGTTTGTTTCCGGTTATTTTACGC
ATAAGC
oCARBO10083:
(SEQ ID NO: 40)
CTCTCCCATATGGTCGACCTGCAGGCGGCCGATAACTGAT
GAAATACGC
oCARBO10189:
(SEQ ID NO: 41)
TCATAATACGTCAGCCCTGTC
oCARBO10190:
(SEQ ID NO: 42)
ATAACTGATGAAATACGCTTCTG
P202:
(SEQ ID NO: 43)
CAGCCTCTTCTAGTCCGCTCACAATTCC
oCARBO10386:
(SEQ ID NO: 44)
GCGTTCGGTTTCAGAATC

Primers and their sequences used for the generation and check of a strain wherein the a3 gene (SEQ ID NO: 104) is deleted.

oCARBO10084:
(SEQ ID NO: 45)
TTCTTATCGGAGGTTTTATATACGTGGCCGCCTGLAGGTC
GACCATATGG
oCARBO10085:
(SEQ ID NO: 46)
ATAGTGCTTTCCCTAGCTTGAAATCCCGCGGCCATGGCGG
CCGGGAGCATG
oCARBO10086:
(SEQ ID NO: 47)
GCCGCCATGGCCGCGGGATTTCAAGCTAGGGAAAGCACTA
TC
oCARBO10087:
(SEQ ID NO: 48)
CTTCATTGATCAGAATTCGAACACTAGACAATGTTGGAGA
GGTTAGGCACTATTGG
oCarboT0088:
(SEQ ID NO: 49)
GTGCCTAACCTCTCCAACATTGTCTAGTGTTCGAATTCTG
ATCAATGAAG
oCARBO10089:
(SEQ ID NO: 50)
ATGGCGTGGTATATGCTTGAAATAACTGGAAACAAACGAC
CCAACACC
oCARBO10090:
(SEQ ID NO: 51)
AACGCACGGGTGTTGGGTCGTTTGTTTCCAGTTATTTCAA
GCATATACCACGCCATGAG
oCARBO10091:
(SEQ ID NO: 52)
TCTGGAGGATCATGTGTGTTGTCGGCCGCCTGCAGGTCGA
CCATATG
oCARBO10191:
(SEQ ID NO: 53)
TCAAGCTAGGGAAAGCACTATC
oCARBO10192:
(SEQ ID NO: 54)
ACGTATATAAAACCTCCGATAAG
oCARBO10384:
(SEQ ID NO: 55)
AGCGGTGTACAGGTTAGG
p202:
(SEQ ID NO: 43)
CAGCCTCTTCTAGTCCGCTCACAATTCC

Primers and their sequences used for the generation and check of a strain wherein the a4 gene (SEQ ID NO: 106) is deleted.

oCARBO10094:
(SEQ ID NO: 56)
TCTGGAGGATCATGTGTGTTGTCGGCCGCCTGCAGGTCGA
CCATATG
oCARBO10095:
(SEQ ID NO: 57)
AAAGTGCCGAGCACCCCTTACAGAATCCCGCGGCCATGGC
GGCCGGGAGCATG
oCARBO10096:
(SEQ ID NO: 58)
GCCGCCATGGCCGCGGGATTCTGTAAGGGGTGCTCGGCAC
TTTC
oCARBO10098_HR1E6:
(SEQ ID NO: 59)
CATTGATCAGAATTCGAACACTGGGCTGTGTTGAACAGTT
TAGACAC
oCARBO10099:
(SEQ ID NO: 60)
GAGTGTCTAAACTGTTCAACACAGCCCAGTGTTCGAATTC
TGATC
oCARBO10100:
(SEQ ID NO: 61)
GACATATGTCCAAAGCTACAGGGAACAAACGACCCAACAC
CCGTGCGTTTTATTC
oCARBO10101:
(SEQ ID NO: 62)
ACGGGTGTTGGGTCGTTTGTTCCCTGTAGCTTTGGACATA
TGTC
oCARBO10102:
(SEQ ID NO: 63)
CCATATGGTCGACCTGCAGGCGGCCGACAACACACATGAT
CCTCCAGAG
oCARBO10178:
(SEQ ID NO: 64)
CTGTAAGGGGTGCTCGGCACTTTC
oCARBO10179:
(SEQ ID NO: 65)
AAACCAACAGTTCCTCGACATATTTTG
P202:
(SEQ ID NO: 43)
CAGCCTCTTCTAGTCCGCTCACAATTCC
oCARBO10388:
(SEQ ID NO: 66)
CTCATGTTGTGGGTTCTG

Primers and their sequences used for the generation and check of a strain wherein the a5 gene (SEQ NO: 108) is deleted.

oCARBO9731:
(SEQ ID NO: 67)
GCAACGATGATATGATTGACGGGAATCCCGCGGCCATGGC
GG
oCARBO9732:
(SEQ ID NO: 68)
CAGACGTCTTGTTCGCTTCGGCCGCCTGCAGGTCGACCAT
ATG
oCARBO9733:
(SEQ ID NO: 69)
CCGCCATGGCCGCGGGATTCCCGTCAATCATATCATCGTT
GC
oCARBO9734:
(SEQ ID NO: 70)
GCCATCATGGTTCAACCTCACGTGAATATAGATTCGAATG
AAAGGAG
oCARBO9735:
(SEQ ID NO: 71)
CTCCTTTCATTCGAATCTATATTCACGTGAGGTTGAACCA
TGATGGC
oCARBO9736:
(SEQ ID NO: 72)
GGGTTGGAAGGCGAAAGAGACCCGGGTATACTAGTGATTT
G
oCARBO9737:
(SEQ ID NO: 73)
CAAATCACTAGTATACCCGGGTCTCTTTCGCCTTCCAACC
C
oCARBO9738:
(SEQ ID NO: 74)
CATATGGTCGACCTGCAGGCGGCCGAAGCGAACAAGACGT
CTG
oCARBO10015:
(SEQ ID NO: 75)
CCCGTCAATCATATCATCGTTGCC
oCARBO10016:
(SEQ ID NO: 76)
GAAGCGAACAAGACGTCTG
oCARBO10118:
(SEQ ID NO: 77)
CAAGATCTCCTCGACACAATGG
p202:
(SEQ ID NO: 43)
CAGCCTCTTCTAGTCCGCTCACAATTCC

Primers and their sequences used for the generation and check of a strain wherein the a6 gene (SEQ ID NO: 110) is deleted.

oCARBO10097:
(SEQ ID NO: 78)
CAGTTTGTAAACTCGGCTCCGAACAGGCCGCCTGCAGGTC
GACCATATGGGAGAG
oCARBO10103:
(SEQ ID NO: 79)
CTGTTGCCCTTCGAGCTGGAAATCCCGCGGCCATGGCGGC
CGGGAGCATG
oCARBO10104:
(SEQ ID NO: 80)
CCGGCCGCCATGGCCGCGGGATTTCCAGCTCGAAGGGCAA
CAGGGTAG
oCARBO10105:
(SEQ ID NO: 81)
TTGATCAGAATTCGAACACTACTTCATCCTTTCCCAACCC
ATGAAATCC
oCARBO10106:
(SEQ ID NO: 82)
GGGTTGGGAAAGGATGAAGTAGTGTTCGAATTCTGATCAA
TGAAG
oCARBO10107:
(SEQ ID NO: 83)
TCTTCCCATTAGATCTTATTTCACAAACAAACGACCCAAC
ACCCGTGCGTTTTATTC
oCARBO10108:
(SEQ ID NO: 84)
ACGCACGGGTGTTGGGTCGTTTGTTTGTGAAATAAGATCT
AATGGGAAGAG
oCARBO10109:
(SEQ ID NO: 85)
TATGGTCGACCTGCAGGCGGCCTGTTCGGAGCCGAGTTTA
CAAACTG
oCARBO10193:
(SEQ ID NO: 86)
TCCAGCTCGAAGGGCAACAG
oCARBO10194:
(SEQ ID NO: 87)
TGTTCGGAGCCGAGTTTAC
P202:
(SEQ ID NO: 43)
CAGCCTCTTCTAGTCCGCTCACAATTCC
oCARBO10904:
(SEQ ID NO: 88)
GTCTTGGTTAGAACGTGAGCAGATA

Primers and their sequences used for the generation and check of a strain wherein the a7 gene (SEQ ID NO: 112) is deleted.

oCARBO10217:
(SEQ ID NO: 89)
GGCTGCAATGTCTCTTCTCGATACTTACGGCCGCCTGCAG
GTCGACCATATG
0CARBU1U218:
(SEQ ID NO: 90)
TGGTGTCCGCGGGCACAATTTCTTAATCCCGCGGCCATGG
CGGCCGGGAG
oCARBO10219:
(SEQ ID NO: 91)
CATGCTCCCGGCCGCCATGGCCGCGGGATTAAGAAATTGT
GCCCGCGGAC
oCARBO10220:
(SEQ ID NO: 92)
GTGTACTTCATTGATCAGAATTCGAACACTACTTCATCCC
TTCCCAACCCATGAAATCC
oCARBO10221:
(SEQ ID NO: 93)
GGATTTCATGGGTTGGGAAGGGATGAAGTAGTGTTCGAAT
TCTGATCAATG
oCARBO10222:
(SEQ ID NO: 94)
GTGGCTCTTCCCATTAGATCGTATTTCACGAACAAACGAC
CCAACAC
oCARBO10223:
(SEQ ID NO: 95)
ACGCACGGGTGTTGGGTCGTTTGTTCGTGAAATACGATCT
AATGGGAAGAG
oCARBO10224:
(SEQ ID NO: 96)
CCATATGGTCGACCTGCAGGCGGCCGTAAGTATCGAGAAG
AGACATTG
oCARBO11493:
(SEQ ID NO: 97)
AAGAAATTGTGCCCGCGGAC
oCARBO11494_N3CASS_REV:
(SEQ ID NO: 98)
GTAAGTATCGAGAAGAGACATTG
oCARBO11495:
(SEQ ID NO: 99)
AGCTGTAGTATTTGACTTC
p202:
(SEQ ID NO: 43)
CAGCCTCTTCTAGTCCGCTCACAATTCC

### Analytical Techniques

Follow-Up of Growth

Optical density (OD) of cultures was measured at 600 nm using the Jasco V 630 Bio spectrophotometer (Jasco Europe) of 1 mL samples diluted with physiological solution (9 g/L NaCl). The viability of yeast cells in cultivation experiments was assessed by determining colony forming units (CFUs) which were expressed as the average logarithm of CFUs per culture volume as log(CFU/mL) (Saerens, Saey, et al., 2011). Alternatively, Cell Dry Weight (CDW) was determined by centrifuging 1 mL fermentation samples at 14000 rpm for 5 min in tared Eppendorf tubes, and subsequently washing the cell pellets twice with 1 mL physiological solution. The remaining cell pellet was put into a 70° C. oven for 5 days and then weighed afterwards. The CDW (g/L) was calculated after subtraction of the empty weight of the Eppendorf tube.

Follow-Up of Glucose Concentration

Glucose concentrations were determined using the 2700 Select Biochemistry Analyser (YSI Inc.) or using Ultra Performance Liquid Chromatography (Waters Acquity H-Class UPLC), coupled with an Evaporative Light Scattering Detector (Waters Acquity ELSD Detector) (UPLC-ELSD). For the UPLC analysis, an Acquity UPLC BEH Amide column (130 Å, 1.7 μm, 2.1×100 mm) (Waters) was used at 35° C. and an isocratic flow rate of 0.5 mL/min of 75% acetonitrile and 0.2% triethylamine (TEA) was applied (5 min/sample). For the ELS detection, the nebulizer was cooled to 15° C. and the drift tube was kept at a temperature of 50° C. The linear range was found to lie between 0 and 5 g/L glucose, using a gain of 100 for ELS detection (Empower software). To express glucose consumption, a linear curve was fitted through the obtained glucose concentrations by UPLC-ELSD, and the respective slope was taken and denoted as the glucose consumption rate (g/L·h).

Analysis of Glycolipids/Glycosides

Samples for glycolipid analysis were prepared by shaking a mixture of 1 mL of pure ethanol and 0.5 mL of fermentation broth vigorously for 5 minutes. Subsequently, after centrifugation for 5 minutes at 15000 rpm, the cell pellet was removed and the supernatant was filtered using a PTFE filter (cut-off 0.22 μm, Novolab) and adequately diluted in 50% ethanol (unless stated otherwise) before analyzing on (Ultra) High Pressure Liquid Chromatography-Mass Spectrometry ((U)HPLC-MS) and (U)HPLC-ELSD (Evaporative Light Scattering Detector).

HPLC-MS was performed using an LC (Shimadzu), coupled to an MS (Micromass Quattro LC) detection system. The different components were separated by polarity on a Chromolith Performance RP-18 Endcapped 100-4.6 mm column (Merck KGaA) at 30° C. The LC-MS method uses a gradient elution based on two solvents: MilliQ with 0.5% acetic acid, and pure acetonitrile (ACN). During the analysis, a flow rate of 1 mL/min was applied. The gradient starts with 5% acetonitrile and increases linearly until 95% over the course of 40 min. After this, the 95% acetonitrile is held for another 10 min, after which this is brought back to 5% acetonitrile in 5 min. The total analysis time per sample is 60 min/sample. The scanning range of the MS was set to 215-1100 g/mol. Using similar conditions as mentioned for HPLC-MS, HPLC-ELSD analysis was performed by Varian Prostar HPLC (ThermoScientific), coupled with an 2000ES ELSD (Alltech) at 40° C. All other conditions are similar as mentioned for the HPLC-MS.

UPLC-ELSD analysis was performed on a Acquity H-Class UPLC (Waters) and Acquity ELSD Detector (Waters), employing the same column and analysis method as UPLC-MS. For the ELSD detection, the nebulizer was cooled at 12° C. and the drift tube was kept at a temperature of 50° C., the gain was set to 200. To quantify the glycolipids, a dilution series of purified product was prepared. An available C18:1 acetylated bola SS purified batch (batch number SL24A) and purified acetylated C16:0 alkyl SS batch (batch number aAlkC16_2) were employed for quantification of bola SS and alkyl SS, respectively.

Alternatively, UPLC-MS was performed with an Accela (ThermoFisher Scientific) and Exactive Plus Orbitrap Mass Spectrometer (ThermoFisher Scientific). For glycolipid analysis, an Acquity UPLC CSH C18 column (130 Å, 1.7 μm, 2.1 mm×50 mm) (Waters) and a gradient elution system based on 0.5% acetic acid in milliQ (A) and 100% acetonitrile (B) at a flow rate of 0.6 mL/min was applied. The method was as follows: the initial concentration of 5% B (95% A) increases linearly until 95% B (5% A) during the first 6.8 min, and then linearly decreases again to 5% B (95% A) during 1.8 min. Subsequently, 5% B (95% A) is maintained until the end of the run (10 min/sample). Negative ion mode was used, and 2 μL samples were injected. MS detection occurred with a Heated Electrospray Ionization (HESI) source and conditions were set to detect masses ranging from 215-1300 m/z in a qualitative way.

All ¹H and ¹³C NMR spectra were recorded at 400 and 100.6 MHz, respectively, on a Bruker Avance III, equipped with ¹H/BB z-gradient probe (BBO, 5 mm). DMSO-[D6] was used as solvent, and as internal chemical shift standard (2.50 ppm for ¹H and 39.52 ppm for ¹³C). All spectra were processed using TOPSPIN 3.2 software. Attached Proton Test (APT), ¹³C, COSY, and HSQC spectra were acquired through the standard sequences available in the Bruker pulse program library. Custom settings were used for HMBC (32 scans), TOCSY (100 millisec MLEV spinlock, 0.1 sec mixing time, 1.27 sec relaxation delay, 16 scans), and H2BC (21.8 millisec mixing time, 1.5 sec relaxation delay, 16 scans), according to literature (Gheysen et al., 2008; Petersen et al., 2006).

Results

Generation of Deletion Strain Δcyp52M1 Δfao1 α1 Δa2 Δa3 Δa4 Δa5 Δa6 Δa7

The Δcyp52M1 Δfao1 strain described under example 1 was used as a parental strain. This strain was made ura3 negative again by selectively removing the ura3 marker again by transforming the ura3 positive Δcyp52M1Δfao1 strain with a ura3 PT recovery cassette consisting of the ura3 promotor (P) fused to the ura3 terminator (T) and selecting for ura3 negative colonies as described by Van Bogaert et al. (2007).

Subsequently the ura3 negative Δcyp52M1 Δfao1 strain was further modified to knock out the a1 to a7 genes one by one. These respective knock out cassettes were all generated using the ura3 gene as a selection marker and following a parallel workflow. When a novel S. bombicola knock out strain was generated (ura3 positive), the resulting strain was then used for a subsequent deletion round using the ura3 gene again as a selection marker. This allowed the use of a vector system as shown in FIG. 15, to generate the specific knock out cassettes of all seven oxidizing enzymes A1 to A7.

Linear knock-out cassettes were generated from the generated plasmids and the linear DNA (approximately 1000 ng) was used to transform the specific S. bombicola strain, which was subsequently plated on the appropriate selective medium as described in materials and methods. For the deletion strains transformed with the a1, a2, a3, a4, a5, a6 and/or a7 knock-out cassettes, selection for ura3 positive strains occurred as described by Van Bogaert et al. (2007). After incubation at 30° C. multiple colonies appeared on the plates. Ten colonies from each transformed strain were picked-up from the plates and Yeast Colony PCR as described by Lodens et al. (2018). The primer sets used to confirm the deletion of the genes of interest (incorporation of the knock-out cassettes) were for a1: oCARBO10025 and oCARBO10026, for a2: P202 and oCARBO10386, for a3: p202 and oCARBO10384, for a4: P202 and oCARBO10388, for a5: p202 and oCARBO10118, for a6: P202 and oCARBO10904 and for a7: p202 and oCARBO11495. Evaluation of novel S. bombicola strains

The novel S. bombicola strains generated and described above (in each transformation step, a novel S. bombicola strain was generated) were evaluated for a number of parameters as described under the materials section. The general characteristics linked to growth and overall viability of the Δcyp52M1Δfao1Δa3Δa4 and the Δcyp52M1Δfao1Δa1Δa3Δa4 strains remained similar to the parental strains PT36; Δcyp52M1 and Δcyp52M1Δfao1.

The glycoside/glycolipid production profile varied among the strains. The Δcyp52M1 strain did not produce glycolipids/glycosides detectable by UPLC-ELSD or oils, fatty acids or fatty alcohols, while the Δcyp52M1Δfao1 strain produced considerable amounts of (acetylated) α,ω-bola sophorosides when fed of fatty alcohols as described under example 1 and shown in FIG. 17A. Several of the novel generated S. bombicola strains additionally deleted in one or more of the a1 to a7 genes, in particular the strain wherein the a3 and a4 genes were deleted, were characterized by enriched production profiles of the alkyl sophorosides (see FIG. 17B for the Δcyp52M1Δfao1Δa3Δa4 strain and FIG. 17C for the Δcyp52M1Δfao1Δa1Δa3Δa4 strain). The production profile of the Δcyp52M1Δfao1Δa1Δa3Δa4 strain (UPLC-ELSD chromatogram depicted in FIG. 17C) further differed from the production profile of the Δcyp52M1Δfao1 strain in the absence of the C18:1 bola SSs which eluted between 3 and 4.5 min for the Δcyp52M1Δfao1 strain. Instead, mono-acetylated C18:1 alkyl SSs (634 g/mol at 5.75 min) formed the most dominant product peak. The presence of non- and di-acetylated C18:1 alkyl SSs (592 and 676 g/mol respectively) was confirmed by HPLC-MS as well. Acetylated and non-acetylated alkyl sophorosides with other chain lengths were also detected. The alkyl chain of the produced alkyl sophorosides in the growth experiments corresponds to the chain length of the specific fatty alcohol that was fed to the production medium.

Additional deletion of the ugtB1 gene in the resulting knock out strain(s) gave rise to the production of corresponding alkyl glucosides instead of alkyl sophorosides.

### Example 4: Optimization of Production of (Acetylated) C9:0 ω-Sophoroside Aldehydes, C9:0 ω-Sophorolipids and C9:0 ω-Sophoroside Alcohols and Production of (Acetylated) C9:0 ω-Glucosides

Materials and Methods

Chemicals and Precursors

The chemical reagents (Oxone®(2KHSO₃·KHSO₄·K₂SO₄), DMSO-d₆, MeOD-d₄) were purchased from Sigma Aldrich and the phosphate salts (Na₂HPO₄·2H₂O, NaH₂PO₄·2H₂O) were purchased from Acros Organics and VWR Chemicals, respectively. Catazyme® 25 L was purchased from Novozymes. They were used without further purification. Oxygen (99.9%, Air Liquide) was used as starting material for the generation of ozone. The acetylated and non-acetylated C18:1 symmetrical α,ω-bola sophorosides (C18:1) were produced as described in example 1.

Production of C9:0 ω-Sophoroside Aldehydes Via Ozonolysis in the Presence of Catalase at Small Pilot Scale

The ozonolysis reaction at small pilot scale was carried out in a stainless-steel 7 L reactor as described above in Example 2. To suppress the overoxidation of C9:0 ω-sophoroside aldehydes by H₂O₂ to C9:0 ω-sophorolipid acids, catalase solution (Catazyme® 25 L) (10 equivalents related to the in-situ formed H₂O₂) was added to the reaction mixture prior to the experiment. To ensure that the pH was optimal for the catalase enzyme, the ozonolysis was carried out in a 2 L buffer solution (pH=6.9-7.1). An overview of the reaction parameters is presented in Table 5.

Production of Diacetylated and Nonacetylated ω C9:0 Sophorolipid Via Prolonged Ozonolysis

Diacetylated and nonacetylated C9:0 ω-sophorolipid acids were produced via prolonged ozonolysis of the diacetylated or non-acetylated symmetrical C18:1 α,ω-bola sophorosides at lab-scale as well as at small pilot-scale. The same set of equipment was used as described above for the production of C9:0 ω-sophoroside aldehydes via ozonolysis. The reaction parameters are shown in Table 7.

Production of Diacetylated and Nonacetylated ω C9:0 Sophorolipid Via Oxidation by Oxone®

Diacetylated and nonacetylated C9:0 ω-sophoroside aldehydes were subjected to oxidation with Oxone® to obtain corresponding C9:0 ω-sophorolipids. Briefly, diacetylated or nonacetylated C9:0 ω-sophoroside aldehyde (1 eq) was dissolved in 25 mL demineralized water in a 50 mL flask. Oxone (0.5 eq) was added to the reaction mixture and it was stirred overnight (18 h) at room temperature. After that, the reaction mixture was concentrated under reduced pressure and re-dissolved in methanol to precipitate the Oxone® salts. Upon filtration of the salts, the filtrate was concentrated under reduced pressure.

Production of Diacetylated and Non-Acetylated ω C9:0 Sophoroside Alcohol

Di-acetylated and non-acetylated C9:0 ω-sophoroside aldehydes were subjected to reduction with picoline-borane (synthesized following the procedure by Kulkarni & Ramachandran (2017)) as a reducing agent to obtain the corresponding C9:0 ω-sophoroside alcohols. Briefly, di-acetylated or non-acetylated C9:0 ω-sophoroside aldehyde (1 eq) was dissolved in 15 mL demineralized water in a 50 mL flask. Picoline-borane (1 eq) was added to the reaction mixture which was acidified with acetic acid to pH=6. The reaction mixture was stirred overnight (18 h) at room temperature. Afterwards, the reaction medium was washed with toluene. The aqueous phase was concentrated under reduced pressure and the C9:0 ω-sophoroside alcohols were obtained as a white powder.

Production of ω (Acetylated) C9:0 ω-Glucoside Aldehydes and C9:0 ω-Glucolipids at Laboratory Scale

(Acetylated) C18:1 symmetrical α,ω-bola glucoside (C18:1) were produced as described in example 1 and used as the substrate to produce C9:0 α,ω-glucosides via ozonolysis. The same lab-scale ozonolysis equipment was used as described above for the production C9:0 α,ω-sophorosides. Briefly, 0.52 g of substrate dissolved in 0.06 L of demineralized water was subjected to ozonolysis (ozone delivery: 2.4 mg min-1) in a gas-washing bottle at room temperature. The solution was mixed at 600 rpm during the experiment. The pH of the reaction mixture was maintained between 8-10 throughout the experiment by adding 0.1 N aqueous NaOH solution at certain intervals. The experiment was ended after 19 min. The product solution was analyzed by 1H NMR and HPLC-MS.

Analytical Procedures

NMR spectroscopy and LC-MS chromatography were performed as described in Example 2.

Results

Production of Diacetylated and Nonacetylated ω C9:0 Sophoroside Aldehyde

To mitigate the overoxidation of C9:0 ω-sophoroside aldehyde to C9:0 ω-sophorolipid by the in-situ formed H₂O₂ during ozonolysis, the ozonolysis reaction was carried out in the presence of catalase enzyme. The reaction parameters employed in these experiments can be seen in Table 5. The results are shown in Table 6.

TABLE 5
Overview of the experimental parameters used for the small pilot-
scale ozonolysis experiments (with catalase addition).

small pilot-scale

| | diacetylated | non-acetylated |
|---|---|---|
| parameters | bola SS | bola SS |
| concentration of substrate | 12.5 | 12.5 |
| (aq.)(g L⁻¹) | | |
| volume of substrate solution (L) | 2.0 | 2.0 |
| O₂/O₃ flow rate (L min⁻¹) | 1.0 | 1.0 |
| O₃ delivery (mg min⁻¹) | 55 | 55 |
| reaction time (min) | 20 | 19 |
| temperature (° C.) | 25 | 25 |
| mixing (rpm) | 200 | 200 |

An increase in the selectivity of the C9:0 ω-sophoroside aldehyde occurred (Table 6) when catalase was present in the reaction medium.

TABLE 6
Relative contents of C9:0 ω-sophoroside aldehyde and
C9:0 ω-sophorolipid in the reaction mixture, after
applying ozonolysis conditions on (acetylated: R = Ac) C18:1
α,ω-bola sophorosides at small pilot-scale (with catalase
addition), as determined by ¹H NMR.

| | | | C9:0 ω-sophoroside | |
|---|---|---|---|---|
| entry | substrate | catalase | aldehyde | C9:0 ω-sophorolipid |
| 5 | R = Ac | + | 81.1 | 18.9 |
| 6 | R = H | + | 84.3 | 15.6 |

Production of Diacetylated and Nonacetylated ω C9:0 Sophorolipid

(i) Via Oxidation of C9:0 ω-Sophoroside Aldehyde by Oxone®

The C9:0 ω-sophoroside aldehyde obtained at the end of ozonolysis was subjected to oxidation using Oxone® as an oxidant. Advantageously, the reaction can be carried out immediately after ozonolysis without removing the water. Both diacetylated and non-acetylated ω C9:0 sophorolipids were obtained in 98% yield starting from the corresponding C9:0 ω-sophoroside aldehydes.

(ii) Via Prolonged Ozonolysis of C18:1 α,ω-Bola Sophorosides

Alternatively, by extending the ozonolysis time, the oxidation of the formed C9:0 ω-sophoroside aldehydes into C9:0 ω-sophorolipids. The reaction parameters corresponding to the prolonged ozonolysis experiments at lab-scale and small pilot-scale are presented in Table 7.

TABLE 7
Overview of the experimental parameters used during
prolonged ozonolysis experiments at lab-scale and
small pilot-scale for C9:0 ω-sophorolipid production.

| | lab-scale | | small pilot-scale | |
|---|---|---|---|---|
| | diacety- | non- | diacety- | non- |
| | lated | acetylated | lated | acetylated |
| Parameters | bola SS | bola SS | bola SS | bola SS |
| concentration of substrate | 20 | 20 | 12.5 | 7.5 |
| (aq.) (g L⁻¹) | | | | |
| volume of substrate | 0.1 | 0.1 | 2.0 | 2.0 |
| solution (L) | | | | |
| O₂/O₃ flow rate (L min⁻¹) | 0.11 | 0.08 | 1.5 | 1.0 |
| O₃ delivery (mg min⁻¹) | 6.4 | 4.6 | 82.5 | 55 |
| reaction time (hours) | 11 | 8.6 | 3.3 | 3.8 |
| temperature (° C.) | 25 | 25 | 25 | 25 |
| mixing (rpm) | 600 | 600 | 200 | 200 |

In the lab-scale experiments, the required ozone quantities for the complete conversion of diacetylated and non-acetylated C9:0 ω-sophoroside aldehydes were 48 and 22 equivalents related to the corresponding C18:1 α,ω-bola sophorosides. Deacetylation of the diacetylated C9:0 ω-sophoroside aldehyde might have occurred during the extended ozonolysis, resulting in a higher consumption of ozone. In the case of pilot-scale experiments, 15 and 16 equivalents of ozone were required for the complete conversion of diacetylated and non-acetylated C9:0 ω-sophoroside aldehydes, respectively. At the end of these prolonged ozonolysis experiments, C9:0 ω-sophoroside aldehydes were converted into the corresponding ω C9:0 sophorolipids, which appeared as a sticky off-white gel after freeze drying of the reaction mixture.

Production of Diacetylated and Nonacetylated ω C9:0 Sophoroside Alcohol

The C9:0 ω-sophoroside aldehyde obtained at the end of ozonolysis was reduced to the corresponding alcohol using picoline-borane as a reducing agent. Diacetylated and nonacetylated ω C9:0 sophoroside alcohols were obtained as a white powder after washing the reaction mixture with toluene followed by evaporating the aqueous phase under reduced pressure. The diacetylated and nonacetylated w C9:0 sophoroside alcohols were obtained at yields of 81% and 97%, respectively.

Production of C9:0 α,ω-Glucosides at Laboratory-Scale

C18:1 α,ω-bola glucoside with a varying acetylation degree was subjected to ozonolysis in demineralized water at laboratory-scale. At the end of the ozonolysis experiment, the end point sample and the starting material were compared through ¹H NMR. (see FIG. 18). This comparison shows that the double bond present in the starting material (α,ω C18:1 bola glucoside) was cleaved and C18:1 molecules thus converted with the formation of the C9:0 ω-glucoside aldehyde and C9:0 ω-glucolipid acid molecules as schematically shown in FIG. 18.

### Example 5: Enzymatic Cleavage of Unsaturated Aliphatic Bounds in Unsaturated α,ω-Bola Glycosides

As schematically shown in FIG. 19, a two-step enzymatic route is applied for cleaving the double bound in an unsaturated α,ω-bola glycoside: in a first step, a hydroperoxide (HPO) functional group is introduced on the double bond of the unsaturated alkyl chain by soybean lipoxygenase-1 (SBLO-1) (Clapp et al. (2006)). In a subsequent step, the hydroperoxide is further converted by a hydroxyperoxide lyase (Stolterfoht et al. (2019)) with the formation of a hemiacetal, which disintegrates spontaneously under acidic conditions.

### Example 6: Enzymatic Cleavage of Unsaturated Aliphatic Bounds in Unsaturated α,ω-Bola Glycosides

As schematically shown in FIG. 20, a three-step enzymatic route is applied for cleaving the double bound in an unsaturated α,ω-bola glycoside: in a first step the aliphatic alkene is converted into an epoxyalkane using a biocatalytic system containing styrene monooxygenase from Rhodococcus sp. (Toda et al. (2015)). In a next step, an epoxide hydrolase (Archelas et al. (2016)) is used for opening the oxirane rings, thereby generating a vicinal diol. In a subsequent step, the C—C bound in the vicinal diol is cleaved by the oxidizing enzyme cytochrome P450 enzyme (Ortiz (2005)).

Statements

The present invention is in particular captured by any one or any combination of one or more of the below numbered aspects and embodiments with any other aspects, statement and/or embodiments:

Aspect 1: A method to produce ω-glycosides which contain less than 10% ω-1 glycosides, ω-2 glycosides and/or ω-3 glycosides comprising the steps of:
a. conversion of (a) suitable substrate(s) with a suitable microbial strain to produce a broth comprising unsaturated α,ω-bola glycosides which contain less than 10% of α,ω-1 bola glycosides, α,ω-2 bola glycosides and/or α,ω-3 bola glycosides,
b. optionally purifying said unsaturated α,ω-bola glycosides from the broth of step a), and
c. subjecting said unsaturated α,ω-bola glycosides within said broth produced in step a) to a reaction that breaks at least one unsaturated cleavable aliphatic bond within said unsaturated α,ω-bola glycosides, or, subjecting said unsaturated α,ω-bola glycosides which are purified in step b) to a reaction that breaks at least one unsaturated cleavable aliphatic bond within said unsaturated α,ω-bola glycosides.

Aspect 2: The method according to aspect 1, wherein said reaction that breaks at least one unsaturated cleavable aliphatic bound is an ozonolysis reaction or an enzymatic reaction.

Aspect 3: The method according to aspect 2, wherein said enzymatic reaction is mediated by a lipoxygenase, a hydroxyperoxide lyase, a monooxygenase, a peroxidase/monooxygenase, an epoxide hydrolase, an alcohol dehydrogenase/monooxygenase, or any combination thereof.

Aspect 4: The method according to any one of aspects 1 to 3, wherein said ω-glycosides are ω-sophorosides, ω-glucosides, ω-mannosides, ω-rhamnosides, ω-xylosides, ω-arabinosides, ω-trehalosides, ω-cellobiosides or ω-lactosides.

Aspect 5: The method according to any one of aspects 1 to 4, wherein said ω-glycosides are ω-glycoside aldehydes, ω-glycoside alcohols and/or glycolipids, or derivatives thereof.

Aspect 6: The method according to any one of aspects 1 to 5, wherein said suitable substrate is a combination of a suitable hydrophilic substrate with a suitable hydrophobic substrate, wherein said suitable hydrophilic substrate is selected from the group comprising carbohydrates and polyols, and wherein said suitable hydrophobic substrate is selected from the group comprising alcohols, fatty acids, alkenes and/or alkanes having an aliphatic chain length of at least 6 carbons.

Aspect 7: The method according to any one of aspects 1 to 6, wherein said unsaturated α,ω-bola glycosides are symmetrical.

Aspect 8: The method according to any one of aspects 1 to 7, wherein said microbial strain is a naturally SL producing fungal strain that has been mutated to have a dysfunctional cytochrome P450 monooxygenase CYP52M1 or a homologue thereof and a dysfunctional fatty alcohol oxidase FAO1 or a homologue thereof or is a naturally SL producing fungal strain that has been mutated to have a dysfunctional cytochrome P450 monooxygenase CYP52M1 or a homologue thereof and a dysfunctional fatty alcohol oxidase FAO1 or a homologue thereof and a dysfunctional glucosyltransferase that is responsible for the second glucosylation step in the sophorolipid biosynthetic pathway UGTB1 or a homologue thereof, wherein said SL producing fungal strain is preferably a yeast selected from the group consisting of Starmerella (Candida) bombicola, Starmerella (Candida) apicola, Starmerlla (Candida) magnoliae, Candida gropengiesseri, Starmerella (Candida) batistae, Starmerella (Candida) floricola, Candida riodocensis, Candida tropicalis, Starmerella (Candida) stellata, Starmerella (Candida) kuoi, Candida sp. NRRL Y-27208, Pseudohyphozyma (Rhodotorula, Candida) bogoriensis sp., Wickerhamiella domericqiae and a sophorolipid-producing strain (of the Starmerella clade).

Aspect 9: The method according to any one of aspects 1 to 8, wherein said unsaturated α,ω-bola glycosides are acetylated.

Aspect 10: The method according to any one of aspects 7 to 9, wherein said ω-glycosides are ω-C9 sophorosides or ω-C9 glucosides.

Aspect 11: The method according to any one of aspects 3 to 10, wherein during ozonolysis a protic nucleophile is used as a solvent.

Aspect 12: The method according to aspect 11, wherein said protic nucleophile is water.

Aspect 13: The method according to any one of aspects 1 to 12, further comprising a step of subjecting the ω-glycosides obtained in step c), to a chemical derivatization route selected from the group comprising: acylation, alkylation, amidation, amination, arylation, biotinylation, carbamoylation, carbonylation, cycloaddition, coupling reaction, etherification, esterification, glycosylation, halogenation, metalation, metathesis, nitrile formation, olefination, oxidation, phosphinylation, phosphonylation, phosphorylation, quaternisation, rearrangement reaction, reduction, silylation, thiolation thionation, and combinations thereof.

Aspect 14: A method for the production of alkyl glycosides, said method comprising the conversion of (a) suitable substrate(s) with a suitable microbial strain to produce a broth comprising alkyl glycoside, wherein said microbial strain has been mutated to have a dysfunctional cytochrome P450 monooxygenase CYP52M1 or homologue thereof and a dysfunctional fatty alcohol oxidase FAO1 or a homologue thereof or wherein said microbial strain has been mutated to have a dysfunctional cytochrome P450 monooxygenase CYP52M1, a dysfunctional fatty alcohol oxidase FAO1 or a homologue thereof and a dysfunctional glucosyltransferase that is responsible for the second glucosylation step in the sophorolipid biosynthetic pathway UGTB1 or a homologue thereof, wherein said microbial strain has further been mutated to have at least one dysfunctional oxidizing enzyme responsible for ω-oxidation of long chain fatty alcohols, and wherein said microbial strain is preferably a naturally SL producing fungal strain, more preferably a yeast selected from the group consisting of Starmerella (Candida) bombicola, Starmerella (Candida) apicola, Starmerella (Candida) magnoliae, Candida gropengiesseri, Starmerella (Candida) batistae, Starmerella (Candida) floricola, Candida riodocensis, Candida tropicalis, Starmerella (Candida) stellata, Starmerella (Candida) kuoi, Candida sp. NRRL Y-27208, Pseudohyphozyma (Rhodotorula, Candida) bogoriensis sp., Wickerhamiella domericqiae and a sophorolipid-producing strain of the Starmerella clade.

Aspect 15: The method according to aspect 14, wherein said oxidizing enzyme responsible for ω-oxidation of long chain fatty alcohols selected from the group consisting of: A1 comprising the amino acid sequence set forth in SEQ ID NO:101, A2 comprising the amino acid sequence set forth in SEQ ID NO:103, A3 comprising the amino acid sequence set forth in SEQ ID NO:105, A4 comprising the amino acid sequence set forth in SEQ ID NO:107, A5 comprising the amino acid sequence set forth in SEQ ID NO:109, A6 comprising the amino acid sequence set forth in SEQ ID NO:111 and A7 comprising the amino acid sequence set forth in SEQ ID NO:113.

### REFERENCES

Adlercreutz, P., Svensson, D., Mathew, S., & Karlsson, E. M. N. (2012). Synthesis of long-chain alkyl glycosides (Patent No. EP2401389 Å1).
Anastas, P., & Eghbali, N. (2010). Green chemistry: Principles and practice. Chemical Society Reviews, 39(1), 301-312. https://doi.org/10.1039/b918763b
Archelas, A, Lacazio, G. And Kotik, M. (2016). Epoxide hydrolase and its application in organic synthesis. Green Biocatalysis, 2016, 978-1-118-82229-6.
Asmer, H.-J., Lang, S., Wagner, F., & Wray, V. (1988). Microbial production, structure elucidation and bioconversion of sophorose lipids. Journal of the American Oil Chemists'Society, 65(9), 1460-1466. https://doi.org/10.1007/BF02898308
Baccile, N., Selmane, M., Le Griel, P., Prévost, S., Perez, J., Stevens, C. V., Delbeke, E., Zibek, S., Guenther, M., Soetaert, W., Van Bogaert, I. N. A., & Roelants, S. (2016). pH-Driven Self-Assembly of Acidic Microbial Glycolipids. Langmuir, 32(25), 6343-6359. https://doi.org/10.1021/acs.langmuir.6b00488
Cavalero, D. A., & Cooper, D. G. (2003). The effect of medium composition on the structure and physical state of sophorolipids produced by Candida bombicola ATCC 22214. Journal of Biotechnology. https://doi.org/10.1016/S0168-1656(03)00067-1
Chen, J., Song, X., Zhang, H., Qu, Y., & Miao, J. (2006). Sophorolipid produced from the new yeast strain Wickerhamiella domercqiae induces apoptosis in H7402 human liver cancer cells. Applied Microbiology and Biotechnology, 72(1), 52-59. https://doi.org/10.1007/s00253-005-0243-z
Cifré, L. C., Alemany, M., de Mendoza, D., & Altabe, S. (2013). Exploring the biosynthesis of unsaturated fatty acids in Bacillus cereus ATCC 14579 and functional characterization of novel acyl-lipid desaturases. Applied and Environmental Microbiology, 79(20), 6271-6279. https://doi.org/10.1128/AEM.01761-13
Clapp, C., Strulson, M., Rodriguez, P., Novak, M. (2006). Oxygenation of Monounsaturated Fatty Acids by Soybean Lipoxygenase-1: Evidence for Transient Hydroperoxide Formation. Biochemistry, 45(51):15884-92. DOI: 10.1021/bi0619425
Davila, A.-M., Marchal, R., & Vandecasteele, J. P. (1994). Sophorose lipid production from lipidic precursors: Predictive evaluation of industrial substrates. Journal of Industrial Microbiology, 13(4), 249-257. https://doi.org/10.1007/BF01569757
Davila, A. M., Marchal, R., Monin, N., & Vandecasteele, J. P. (1993). Identification and determination of individual sophorolipids in fermentation products by gradient elution high-performance liquid chromatography with evaporative light-scattering detection. Journal of Chromatography A, 648(1), 139-149. https://doi.org/10.1016/0021-9673(93)83295-4
Delbeke, E. I. P. (2016). Innovative sophorolipid analogues with tailor-made physico-chemical and biological properties. Ghent University.
Delbeke, E. I. P., Everaert, J., Lozach, O., Le Gall, T., Berchel, M., Montier, T., Jaffrès, P. A., Rigole, P., Coenye, T., Brennich, M., Baccile, N., Roelants, S. L. K. W., Soetaert, W., Van Bogaert, I. N. A., Van Geem, K. M., & Stevens, C. V. (2018). Synthesis and Biological Evaluation of Bolaamphiphilic Sophorolipids. ACS Sustainable Chemistry and Engineering, 6(7), 8992-9005. https://doi.org/10.1021/acssuschemeng.8b01354
Delbeke, E. I. P., Lozach, O., Le Gall, T., Berchel, M., Montier, T., Jaffrès, P. A., Van Geem, K. M., & Stevens, C. V. (2016). Evaluation of the transfection efficacies of quaternary ammonium salts prepared from sophorolipids. Organic and Biomolecular Chemistry, 14(15), 3744-3751. https://doi.org/10.1039/c6ob00241b
Delbeke, E. I. P., Movsisyan, M., Van Geem, K. M., & Stevens, C. V. (2015). Chemical and enzymatic modification of sophorolipids. In Green Chemistry (Vol. 18, Issue 1, pp. 76-104). https://doi.org/10.1039/c5gc02187a
Delbeke, E. I. P., Roelants, S. L. K. W., Matthijs, N., Everaert, B., Soetaert, W., Coenye, T., Van Geem, K. M., & Stevens, C. V. (2016). Sophorolipid amine oxide production by a combination of fermentation, scale-up and chemical modification. Industrial and Engineering Chemistry Research, 55(27), 7273-7281. https://doi.org/10.1021/acs.iecr.6b00629
Delbeke, E. I. P., Roman, B. I., Marin, G. B., Van Geem, K. M., & Stevens, C. V. (2015). A new class of antimicrobial biosurfactants: Quaternary ammonium sophorolipids. Green Chemistry, 17(6), 3373-3377. https://doi.org/10.1039/c5gc00120j
Develter, D., & Fleurackers, S. (2008). A method for the production of short chained glycolipids (Patent No. EP 1953 237 A1).
Develter, D., & Fleurackers, S. (2011). Improved sophorolactone production. (Patent No. WO 2012168325 A1).
Develter, D. W. G., & Lauryssen, L. M. L. (2010). Properties and industrial applications of sophorolipids. European Journal of Lipid Science and Technology, 112(6), 628-638. https://doi.org/10.1002/ejlt.200900153
Dolman, B. M., Kaisermann, C., Martin, P. J., & Winterburn, J. B. (2017). Integrated sophorolipid production and gravity separation. Process Biochemistry, 54, 162-171. https://doi.org/10.1016/j.procbio.2016.12.021
Fabbri, P., Velasquez Ochoa, J., Agustin Martinez, G., Degli Esposti, M., Wydra, S., ISI Alexander Schwarz, F., ISI Frank Marscheider-Weidemann, F., & Isi, F. (2018). Top emerging bio-based products, their properties and industrial applications AUTHORS & EDITORS. www.ecologic.eu
Farmer, T. J., & Mascal, M. (2015). Platform Molecules. In Introduction to Chemicals from Biomass: Second Edition (pp. 89-155). https://doi.org/10.1002/9781118714478.ch4
Fatma, Z., Jawed, K., Mattam, A. J., & Yazdani, S. S. (2016). Identification of long chain specific aldehyde reductase and its use in enhanced fatty alcohol production in E. coli. Metabolic Engineering, 37, 35-45. https://doi.org/10.1016/j.ymben.2016.04.003
Frische, R., Hegwein, K., & Volkheimer, J. D. (2003). Process for the recovery of saturated dicarboxylic acids from the ozonolysis of unsaturated fatty acids (Patent No. EP1074540 Å1).
Gao, R., Falkeborg, M., Xu, X., & Guo, Z. (2013). Production of sophorolipids with enhanced volumetric productivity by means of high cell density fermentation. Applied Microbiology and Biotechnology, 97(3), 1103-1111. https://doi.org/10.1007/s00253-012-4399-z
Gheysen, K., Mihai, C., Conrath, K., & Martins, J. C. (2008). Rapid identification of common hexapyranose monosaccharide units by a simple TOCSY matching approach. Chemistry—A European Journal, 14(29), 8869-8878. https://doi.org/10.1002/chem.200801081
Goebel, C. G., Brown, A. C., Oehlschlaeger, H. F., & Rolfes, R. P. (1957). Method of making azelaic acid (U.S. Pat. No. 2,813,113A).
Gorin, P. A. J., Spencer, J. F. T., & Tulloch, A. P. (1961). Hydroxy Fatty Acid Glycosides of Sophorose from Torulopsis magnoliae. Canadian Journal of Chemistry, 39(4), 846-855. https://doi.org/10.1139/v61-104
Green, M. R., & Sambrook, J. (2012). Molecular Cloning: A laboratory manual. Fourth Edition.
Gritz, L., & Davies, J. (1983). Plasmid-encoded hygromycin B resistance: the sequence of hygromycin B phosphotransferase gene and its expression in Escherichia coli and Saccharomyces cerevisiae. Gene, 25(2-3), 179-187. https://doi.org/10.1016/0378-1119(83)90223-8
Gross, R. A., Rengathavasi, T., Koh, A., & Peng, K. (2013). Modified sophorolipids as oil solubilizing agents. (U.S. Pat. No. 9,650,405B2).
Hannen, P., Haeger, H., & Roos, M. (2013). Synthesis Of Omega-amino Carboxylic Acids And Their Esters From Unsaturated Fatty Acid Derivatives.
Huang, F.-C. C., Peter, A., & Schwab, W. (2014). Expression and Characterization of CYP52 Genes Involved in the Biosynthesis of Sophorolipid and Alkane Metabolism from Starmerella bombicola. APPLIED AND ENVIRONMENTAL MICROBIOLOGY, 80(2), 766-776. https://doi.org/10.1128/AEM.02886-13
Imura, T., Masuda, Y., Minamikawa, H., Fukuoka, T., Konishi, M., Morita, T., Sakai, H., Abe, M., & Kitamoto, D. (2010). Enzymatic Conversion of Diacetylated Sophoroselipid into Acetylated Glucoselipid: Surface-Active Properties of Novel Bolaform Biosurfactants. Journal of Oleo Science J. Oleo Sci, 59(9), 495-501. https://www.jstage.jst.go.jp/article/jos/59/9/59_9_495/_pdf
Irfan, M., Glasnov, T. N., & Kappe, C. O. (2011). Continuous flow ozonolysis in a laboratory scale reactor. Organic Letters, 13(5), 984-987. https://doi.org/10.1021/ol102984h
Kalim Akhtara, M., Turner, N. J., & Jones, P. R. (2013). Carboxylic acid reductase is a versatile enzyme for the conversion of fatty acids into fuels and chemical commodities. Proceedings of the National Academy of Sciences of the United States of America, 110(1), 87-92. https://doi.org/10.1073/pnas.1216516110
Kang, M. K., & Nielsen, J. (2017). Biobased production of alkanes and alkenes through metabolic engineering of microorganisms. In Journal of Industrial Microbiology and Biotechnology (Vol. 44, Issues 4-5, pp. 613-622). Springer Verlag. https://doi.org/10.1007/s10295-016-1814-y
Kaur, G., Wang, H., To, M. H., Roelants, S. L. K. W., Soetaert, W., & Lin, C. S. K. (2019). Efficient sophorolipids production using food waste. Journal of Cleaner Production, 232, 1-11. https://doi.org/10.1016/j.jclepro.2019.05.326
Kim, K., Yoo, D., Kim, Y., Lee, B., Shin, D., & Kim, E. K. (2002). Characteristics of sophorolipid as an antimicrobial agent. Journal of Microbiology and Biotechnology, 12(2), 235-241.
Köckritz, A., & Martin, A. (2011). Synthesis of azelaic acid from vegetable oil-based feedstocks. European Journal of Lipid Science and Technology, 113(1), 83-91. https://doi.org/10.1002/ejlt.201000117
Konishi, M., Fukuoka, T., Morita, T., Imura, T., & Kitamoto, D. (2008). Production of new types of sophorolipids by Candida batistae. Journal of Oleo Science, 57(6), 359-369. http://www.ncbi.nlm.nih.gov/pubmed/18469499
Kotik, M., Archelas, A., & Wohlgemuth, R. (2012). Epoxide Hydrolases and their Application in Organic Synthesis. Current Organic Chemistry, 16(4), 451-482. https://doi.org/10.2174/138527212799499840
Kurtzman, C. P. (2012). Candida kuoi sp. nov., an anamorphic species of the Starmerella yeast clade that synthesizes sophorolipids. International Journal of Systematic and Evolutionary Microbiology, 62(9), 2307-2311. https://doi.org/10.1099/ijs.0.039479-0
Kurtzman, C. P., Price, N. P. J., Ray, K. J., & Kuo, T. M. (2010). Production of sophorolipid biosurfactants by multiple species of the Starmerella (Candida) bombicola yeast clade. FEMS Microbiology Letters, 311(2), 140-146. https://doi.org/10.1111/j.1574-6968.2010.02082.x
Lang, S., Brakemeier, A., Heckmann, R., Spöckner, S., & Rau, U. (2000). Production of native and modified sophorose lipids. Chimica Oggi, 18(10), 76-79. http://cat.inist.fr/?aModele=afficheN&cpsidt=816695
Li, W., Li, J., & Song, X. (2016). Alkane Utilization, the Expression and Function of Cytochrome P450 in Sophorolipid Synthesis in Starmerella bombicola CGMCC 1576. Research & Reviews: Journal of Microbiology and Biotechnology, 5(2), 58-63.
Lin, S. S., Manchester, J. K., & Gordon, J. I. (2001). Enhanced Gluconeogenesis and Increased Energy Storage as Hallmarks of Aging in Saccharomyces cerevisiae. Journal of Biological Chemistry, 276(38), 36000-36007. https://doi.org/10.1074/jbc.M103509200
Lodens, S., De Graeve, M., Roelants, S. L. K. W., De Maeseneire, S. L., & Soetaert, W. (2018). Transformation of an Exotic Yeast Species into a Platform Organism: A Case Study for Engineering Glycolipid Production in the Yeast Starmerella bombicola. In Methods in molecular biology (Clifton, N.J.) (Braman, J., Vol. 1772, pp. 95-123). Springer. http://www.ncbi.nlm.nih.gov/pubmed/29754224
Lodens, S., Roelants, S. L. K. W., Ciesielska, K., Geys, R., Derynck, E., Maes, K., Pattyn, F., Van Renterghem, L., Mottet, L., Dierickx, S., Vanhaecke, L., Devreese, B., De Maeseneire, S. L., & Soetaert, W. (2020). Unraveling and resolving inefficient glucolipid biosurfactants production through quantitative multiomics analyses of Starmerella bombicola strains. Biotechnology and Bioengineering, 117, 453-465. https://doi.org/10.1002/bit.27191
Lorer, R. (2017). Chemical modification of petroselinic acid-based sophorolipids. Ghent University.
Lourith, N., & Kanlayavattanakul, M. (2009). Natural surfactants used in cosmetics: glycolipids. International Journal of Cosmetic Science, 31(4), 255-261. https://doi.org/10.1111/j.1468-2494.2009.00493.x
McCurry, P. M. J., Varvil, J. R., & Pickens, C. E. (1996). Process for making alkyl polyglycosides (U.S. Pat. No. 5,512,666A).
Mutti, F. G. (2012). Alkene cleavage catalysed by heme and nonheme enzymes: Reaction mechanisms and biocatalytic applications. In Bioinorganic Chemistry and Applications. https://doi.org/10.1155/2012/626909
Nobis, M., & Roberge, D. M. (2011). Mastering ozonolysis: Production from laboratory to ton scale in continuous flow. Chimica Oggi, 29(1), 56-58.
Oehlschlaeger, H. F., & Rodenberg, H. G. (1968). Process for the production of a purified grade of azelaic acid by treatment with ozone during purification (U.S. Pat. No. 3,402,108A).
Omonov, T. S., Kharraz, E., Foley, P., & Curtis, J. M. (2014). The production of biobased nonanal by ozonolysis of fatty acids. RSC Advances, 4(96), 53617-53627. https://doi.org/10.1039/c4ra07917e
Ortiz de Montellano, Paul R. (Ed.) (2005). Cytochrome P450: Structure, Mechanism, and Biochemistry, 3rd ed., edited by Paul R. Ortiz de Montellano. Kluwer Academic/Plenum Publishers, New York
Petersen, B. O., Vinogradov, E., Kay, W., Wurtz, P., Nyberg, N. T., Duus, J., & Sorensen, O. W. (2006). H2BC: A new technique for NMR analysis of complex carbohydrates. Carbohydrate Research, 341(4), 550-556. https://doi.org/10.1016/j.carres.2005.11.020
Philp, J. C., Ritchie, R. J., & Allan, J. E. M. (2013). Biobased chemicals: The convergence of green chemistry with industrial biotechnology. Trends in Biotechnology, 31(4), 219-222. https://doi.org/10.1016/j.tibtech.2012.12.007
Rau, U., Hammen, S., Heckmann, R., Wray, V., & Lang, S. (2001). Sophorolipids: A source for novel compounds. Industrial Crops and Products, 13(2), 85-92. https://doi.org/10.1016/S0926-6690(00)00055-8
Roelants, S. L. K. W., Ciesielska, K., De Maeseneire, S. L., Moens, H., Everaert, B., Verweire, S., Denon, Q., Vanlerberghe, B., Van Bogaert, I. N. A., Van der Meeren, P., Devreese, B., & Soetaert, W. (2016). Towards the industrialization of new biosurfactants: Biotechnological opportunities for the lactone esterase gene from Starmerella bombicola. Biotechnology and Bioengineering, 113(3), 550-559. https://doi.org/10.1002/bit.25815
Roelants, S. L. K. W., De Maeseneire, S. L., Ciesielska, K., Van Bogaert, I. N. A., & Soetaert, W. (2014). Biosurfactant gene clusters in eukaryotes: regulation and biotechnological potential. Applied Microbiology and Biotechnology, 98(8), 3449-3461. https://doi.org/10.1007/s00253-014-5547-4
Roelants, S. L. K. W., Saerens, K. M. J., Derycke, T., Li, B., Lin, Y.-C., Van de Peer, Y., De Maeseneire, S. L., Van Bogaert, I. N. A., & Soetaert, W. (2013). Candida bombicola as a platform organism for the production of tailor-made biomolecules. Biotechnology and Bioengineering, 110(9), 2494-2503. https://doi.org/10.1002/bit.24895
Roelants, S. L. K. W., Solaiman, D. K. Y., Ashby, R. D., Lodens, S., Van Renterghem, L., & Soetaert, W. (2019). Production and Applications of Sophorolipids. In Biobased Surfactants (pp. 65-119). AOCS Press. https://doi.org/10.1016/B978-0-12-812705-6.00003-4 Saerens, K. M. J. (2012). Synthesis of glycolipids by Candida bombicola.
Saerens, K. M. J., Saey, L., & Soetaert, W. (2011). One-step production of unacetylated sophorolipids by an acetyltransferase negative Candida bombicola. Biotechnology and Bioengineering, 108(12), 2923-2931. https://doi.org/10.1002/bit.23248
Saerens, K. M. J., Zhang, J., Saey, L., Van Bogaert, I. N. A., & Soetaert, W. (2011). Cloning and functional characterization of the UDP-glucosyltransferase UgtB1 involved in sophorolipid production by Candida bombicola and creation of a glucolipid-producing yeast strain. Yeast, 28(4), 279-292. https://doi.org/10.1002/yea.1838
Sambrook, J., & Russell, D. W. (David W. (2001). Molecular cloning: a laboratory manual. Cold Spring Harbor Laboratory Press.
Santos, A. R. O., Leon, M. P., Barros, K. O., Freitas, L. F. D., Hughes, A. F. S., Morais, P. B., Lachance, M. A., & Rosa, C. A. (2018). Starmerella camargoi f.A., sp. nov., Starmerella ilheusensis f.a., Sp. Nov., Starmerella litoralis f.a., sp. nov., Starmerella opuntiae f.a., sp. nov., Starmerella roubikii f.a., sp. nov. and Starmerella vitae f.a., sp. nov., isolated from flowers and bee. International Journal of Systematic and Evolutionary Microbiology. https://doi.org/10.1099/ijsem.0.002675
Schiaffo, C. E., & Dussault, P. H. (2008). Ozonolysis in solvent/water mixtures: Direct conversion of alkenes to aldehydes and ketones. Journal of Organic Chemistry, 73(12), 4688-4690. https://doi.org/10.1021/jo800323x
Sen, S., Borah, S. N., Bora, A., & Deka, S. (2017). Production, characterization, and antifungal activity of a biosurfactant produced by Rhodotorula babjevae YS3. Microbial Cell Factories, 16(1), 95. https://doi.org/10.1186/s12934-017-0711-z
Sheldon, R. A. (2014). Green and sustainable manufacture of chemicals from biomass: State of the art. In Green Chemistry (Vol. 16, Issue 3, pp. 950-963). The Royal Society of Chemistry. https://doi.org/10.1039/c3gc41935e
Siebenhaller, S., Grüninger, J., & Syldatk, C. (2018). Enzymatic Synthesis of Glycolipid Surfactants. In Lipid Modification by Enzymes and Engineered Microbes (pp. 293-313). Elsevier. https://doi.org/10.1016/B978-0-12-813167-1.00013-X
Soetaert, W., De Maeseneire, S. L., Saerens, K. M. J., Roelants, S. L. K. W., & Van Bogaert, I. N. A. (2010). Yeast strains modified in their sophorolipid production and uses thereof WO2011154523 A1 (Patent No. WO2011154523 A1). https://www.google.com/patents/WO2011154523 Å1?cl=en&hl=nl
Soetaert, W., Van Bogaert, I., & Roelants, S. (2013). Methods to produce bolaamphiphilic glycolipids. WO 2015028278 Å1. 50p.
Spencer, J. F. T., Gorin, P. A. J., & Tulloch, A. P. (1970). Torulopsis bombicola sp. n. Antonie van Leeuwenhoek, 36(1), 129-133. https://doi.org/10.1007/BF02069014
Stolterfoht, H., Rinnofner, C., Winkler, M., & Pichler, H. (2019). Recombinant Lipoxygenases and Hydroperoxide Lyases for the Synthesis of Green Leaf Volatiles. In Journal of Agricultural and Food Chemistry (Vol. 67, Issue 49, pp. 13367-13392). American Chemical Society. https://doi.org/10.1021/acs.jafc.9b02690
Takahashi, F., Igarashi, K., & Hagihara, H. (2016). Identification of the fatty alcohol oxidase FAO1 from Starmerella bombicola and improved novel glycolipids production in an FAO1 knockout mutant. Applied Microbiology and Biotechnology, 100(22), 9519-9528. https://doi.org/10.1007/s00253-016-7702-6
Takahashi, M., Morita, T., Wada, K., Hirose, N., Fukuoka, T., Imura, T., & Kitamoto, D. (2011). Production of sophorolipid glycolipid biosurfactants from sugarcane molasses using Starmerella bombicola NBRC 10243. Journal of Oleo Science, 60(5), 267-273.
Toda, H., Imae, R., & Itoh, N. (2014). Bioproduction of Chiral Epoxyalkanes using Styrene Monooxygenase from Rhodococcus sp. ST-10 (RhSMO). Advanced Synthesis & Catalysis, 356(16), 3443-3450. https://doi.org/10.1002/adsc.201400383
Toda, H., Ohuchi, T., Imae, R. and Itoh, N. (2015). Microbial Production of Aliphatic (S)-Epoxyalkanes by Using Rhodococcus sp. Strain ST-10 Styrene Monooxygenase Expressed in Organic-Solvent-Tolerant Kocuria rhizophila DC2201. Appl. Environ. Microbiol. 81(6): 1919-1925.
Tran, P., Graiver, D., & Narayan, R. (2005). Ozone-mediated polyol synthesis from soybean oil. JAOCS, Journal of the American Oil Chemists' Society, 82(9), 653-659. https://doi.org/10.1007/s11746-005-1124-z
Tuck, C. O., Perez, E., Horvath, I. T., Sheldon, R. A., & Poliakoff, M. (2012). Valorization of biomass: Deriving more value from waste. In Science (Vol. 337, Issue 6095, pp. 695-699). American Association for the Advancement of Science. https://doi.org/10.1126/science.1218930
Tulloch, A. P., Spencer, J. F. T., & Deinema, M. H. (1968). A new hydroxy fatty acid sophoroside from Candida bogoriensis. Canadian Journal of Chemistry, 46(3), 345-348. https://doi.org/10.1139/v68-057
Tulloch, A. P., Spencer, J. F. T., & Gorin, P. A. J. (1962). THE FERMENTATION OF LONG-CHAIN COMPOUNDS BY TORULOPSIS MAGNOLIAE: I. STRUCTURES OF THE HYDROXY FATTY ACIDS OBTAINED BY THE FERMENTATION OF FATTY ACIDS AND HYDROCARBONS. Canadian Journal of Chemistry, 40(7), 1326-1338. https://doi.org/10.1139/v62-203
Van Bogaert, I. N. A., Buyst, D., Martins, J. C., Roelants, S. L. K. W., & Soetaert, W. K. (2016). Synthesis of bolaform biosurfactants by an engineered Starmerella bombicola yeast. Biotechnology and Bioengineering, 113(12), 2644-2651. https://doi.org/10.1002/bit.26032
Van Bogaert, I. N. A., De Maeseneire, S. L., De Schamphelaire, W., Develter, D., Soetaert, W., & Vandamme, E. J. (2007). Cloning, characterization and functionality of the orotidine-5′-phosphate decarboxylase gene (URA3) of the glycolipid-producing yeast Candida bombicola. Yeast, 24(3), 201-208. https://doi.org/10.1002/yea.1448
Van Bogaert, I. N. A., Demey, M., Develter, D., Soetaert, W., & Vandamme, E. J. (2009). Importance of the cytochrome P450 monooxygenase CYP52 family for the sophorolipid-producing yeast Candida bombicola. FEMS Yeast Research, 9(1), 87-94. https://doi.org/10.1111/j.1567-1364.2008.00454.x
Van Bogaert, I. N. A., Holvoet, K., Roelants, S. L. K. W., Li, B., Lin, Y. C., Van de Peer, Y., & Soetaert, W. (2013). The biosynthetic gene cluster for sophorolipids: A biotechnological interesting biosurfactant produced by Starmerella bombicola. Molecular Microbiology, 88(3), 501-509. https://doi.org/10.1111/mmi.12200
Van Bogaert, I. N. A., Roelants, S., Develter, D., & Soetaert, W. (2010). Sophorolipid production by Candida bombicola on oils with a special fatty acid composition and their consequences on cell viability. Biotechnology Letters, 32(10), 1509-1514. https://doi.org/10.1007/s10529-010-0323-8
Van Bogaert, I. N. A., Zhang, J., & Soetaert, W. (2011). Microbial synthesis of sophorolipids. Process Biochemistry, 46(4), 821-833. https://doi.org/10.1016/j.procbio.2011.01.010
Van Renterghem, L., Roelants, S. L. K. W. K. W., Baccile, N., Uyttersprot, K., Taelman, M. C., Everaert, B., Mincke, S., Ledegen, S., Debrouwer, S., Scholtens, K., Stevens, C., & Soetaert, W. (2018). From lab to market: An integrated bioprocess design approach for new-to-nature biosurfactants produced by Starmerella bombicola. Biotechnology and Bioengineering, 115(5), 1195-1206. https://doi.org/10.1002/bit.26539
Yamagishi, F., Kozuka, Y., Endo, F., & Ooi, H. (1974). Process for synthesizing sucrose esters of fatty acids (U.S. Pat. No. 3,792,041A).

1. A method for the production of alkyl glycosides, said method comprising the conversion of (a) suitable substrate(s) with a suitable microbial strain to produce a broth comprising alkyl glycoside, wherein said microbial strain is a fungal strain that has been mutated to have a dysfunctional cytochrome P450 monooxygenase CYP52M1 or homologue thereof and a dysfunctional fatty alcohol oxidase FAO1 or a homologue thereof or wherein said microbial strain is a fungal strain that has been mutated to have a dysfunctional cytochrome P450 monooxygenase CYP52M1, a dysfunctional fatty alcohol oxidase FAO1 or a homologue thereof and a dysfunctional glucosyltransferase that is responsible for the second glucosylation step in the sophorolipid biosynthetic pathway UGTB1 or a homologue thereof, wherein said fungal strain has further been mutated to have at least one dysfunctional oxidizing enzyme responsible for ω-oxidation of long chain fatty alcohols.

2. The method according to claim 1, wherein said fungal strain is a naturally SL producing fungal strain.

3. The method according to claim 1 or 2, wherein said fungal strain is a yeast selected from the group consisting of Starmerella (Candida) bombicola, Starmerella (Candida) apicola, Starmerella (Candida) magnoliae, Candida gropengiesseri, Starmerella (Candida) batistae, Starmerella (Candida) floricola, Candida riodocensis, Candida tropicalis, Starmerella (Candida) stellata, Starmerella (Candida) kuoi, Candida sp. NRRL Y-27208, Pseudohyphozyma (Rhodotorula, Candida) bogoriensis sp., Wickerhamiella domericqiae and a sophorolipid-producing strain of the Starmerella clade.

4. The method according to any one of claims 1 to 3, wherein said oxidizing enzyme responsible for ω-oxidation of long chain fatty alcohols selected from the group consisting of: A1 comprising the amino acid sequence set forth in SEQ ID NO:101, A2 comprising the amino acid sequence set forth in SEQ ID NO:103, A3 comprising the amino acid sequence set forth in SEQ ID NO:105, A4 comprising the amino acid sequence set forth in SEQ ID NO:107, A5 comprising the amino acid sequence set forth in SEQ ID NO:109, A6 comprising the amino acid sequence set forth in SEQ ID NO:111 and A7 comprising the amino acid sequence set forth in SEQ ID NO:113.

5. The method according to any one of claims 1 to 4, wherein said fungal strain has been mutated to have at least a dysfunctional oxidizing enzyme A3 comprising the amino acid sequence set forth in SEQ ID NO:105 and a dysfunctional oxidizing enzyme A4 comprising the amino acid sequence set forth in SEQ ID NO:107.

6. The method according to any one of claims 1 to 5, wherein said fungal strain has been mutated to have at least a dysfunctional oxidizing enzyme A3 comprising the amino acid sequence set forth in SEQ ID NO:105, a dysfunctional oxidizing enzyme A4 comprising the amino acid sequence set forth in SEQ ID NO:107, and a dysfunctional enzyme A1 comprising the amino acid sequence set forth in SEQ ID NO:101.

7. The method according to any one of claims 1 to 6, wherein said alky glycosides are alkyl glucosides or alkyl sophorosides.

8. A method to produce ω-glycosides which contain less than 10%, preferably less than 1%, ω-1 glycosides, ω-2 glycosides and/or ω-3 glycosides, wherein said ω-glycosides comprise a carbohydrate that is bound to a primary or terminal carbon atom of an aliphatic chain of carbons via a glycosidic bond, comprising the steps of:
a. conversion of (a) suitable substrate(s) with a suitable microbial strain to produce a broth comprising unsaturated α,ω-bola glycosides which contain less than 10%, preferably less than 1%, of α,ω-1 bola glycosides, α,ω-2 bola glycosides and/or α,ω-3 bola glycosides, wherein said microbial strain is a fungal strain that has been mutated to have a dysfunctional cytochrome P450 monooxygenase CYP52M1 or homologue thereof and a dysfunctional fatty alcohol oxidase FAO1 or a homologue thereof or wherein said microbial strain is a fungal strain that has been mutated to have a dysfunctional cytochrome P450 monooxygenase CYP52M1, a dysfunctional fatty alcohol oxidase FAO1 or a homologue thereof and a dysfunctional glucosyltransferase, that is responsible for the second glucosylation step in the sophorolipid biosynthetic pathway UGTB1 or a homologue thereof,
and wherein said unsaturated α,ω-bola glycosides comprise two carbohydrates that are each bound to a primary or terminal carbon atom of an aliphatic chain of carbons via a glycosidic bond, wherein said aliphatic chain of carbons comprises at least one double bond,
b. optionally purifying said unsaturated α,ω-bola glycosides from the broth of step a), and
c. subjecting said unsaturated α,ω-bola glycosides within said broth produced in step a) to an ozonolysis reaction or an enzymatic reaction that breaks at least one unsaturated cleavable aliphatic bond within said unsaturated α,ω-bola glycosides, or, subjecting said unsaturated α,ω-bola glycosides which are purified in step b) to an ozonolysis reaction or an enzymatic reaction that breaks at least one unsaturated cleavable aliphatic bond within said unsaturated α,ω-bola glycosides.

9. The method according to claim 8, wherein said enzymatic reaction is mediated by a lipoxygenase, a hydroxyperoxide lyase, a monooxygenase, a peroxidase/monooxygenase, an epoxide hydrolase, an alcohol dehydrogenase/monooxygenase, a lipase, or any combination thereof, preferably a combination of a lipoxygenase and a hydroxyperoxide lyase or a combination of a monooxygenase, an epoxide hydrolase and an alcohol dehydrogenase/monooxygenase.

10. The method according to any one of claim 8 or 9, wherein said ω-glycosides are ω-sophorosides, or ω-glucosides.

11. The method according to claim 8 to 10, wherein said ω-glycosides are ω-glycoside aldehydes, ω-glycoside alcohols and/or ω-glycolipids, or derivatives thereof.

12. The method according to any one of claims 8 to 11, wherein said suitable substrate is a combination of a suitable hydrophilic substrate with a suitable hydrophobic substrate, wherein said suitable hydrophilic substrate is selected from the group comprising carbohydrates and polyols, and wherein said suitable hydrophobic substrate is selected from the group comprising alcohols, preferably monoalcohols, fatty acids, alkenes and/or alkanes having an aliphatic chain length of at least 6 carbons, preferably wherein said hydrophobic substrate is an unsaturated primary fatty alcohol.

13. The method according to any one of claims 8 to 12, wherein said unsaturated α,ω-bola glycosides are symmetrical.

14. The method according to any one of claims 8 to 13, wherein said fungal strain is a naturally SL producing fungal strain.

15. The method according to any one of claims 8 to 14, wherein said fungal strain is a yeast selected from the group consisting of Starmerella (Candida) bombicola, Starmerella (Candida) apicola, Starmerlla (Candida) magnoliae, Candida gropengiesseri, Starmerella (Candida) batistae, Starmerella (Candida) floricola, Candida riodocensis, Candida tropicalis, Starmerella (Candida) stellata, Starmerella (Candida) kuoi, Candida sp. NRRL Y-27208, Pseudohyphozyma (Rhodotorula, Candida) bogoriensis sp., Wickerhamiella domericqiae and a sophorolipid-producing strain of the Starmerella clade.

16. The method according to any one of claims 8 to 15, wherein said unsaturated α,ω-bola glycosides are acetylated.

17. The method according to any one of claims 13 to 16, wherein said ω-glycosides are ω-C9 sophorosides or ω-C9 glucosides.

18. The method according to any one of claims 8 to 17, wherein during ozonolysis a protic nucleophile is used as a solvent.

19. The method according to claim 18, wherein said protic nucleophile is water.

20. The method according to any one of claims 8 to 19, further comprising a step of subjecting the ω-glycosides obtained in step c), to a chemical derivatization route selected from the group comprising: acylation, alkylation, amidation, amination, arylation, biotinylation, carbamoylation, carbonylation, cycloaddition, coupling reaction, etherification, esterification, glycosylation, halogenation, metalation, metathesis, nitrile formation, olefination, oxidation, phosphinylation, phosphonylation, phosphorylation, quaternisation, rearrangement reaction, reduction, silylation, thiolation thionation, and combinations thereof.

21. Use of an enzyme A1 comprising the amino acid sequence set forth in SEQ ID NO:101 or a homologue thereof, an enzyme A3 comprising the amino acid sequence set forth in SEQ ID NO:105 or a homologue thereof or an enzyme or an enzyme A4 comprising the amino acid sequence set forth in SEQ ID NO:107 or a homologue thereof for the production of diols, preferably α,ω-diols.


SEQUENCE LISTING (113 sequences)

SEQ ID NO: 1 - DNA, 26 bp, Artificial sequence
  Primer
aaggcgggctggaatgcatatctgag

SEQ ID NO: 2 - DNA, 24 bp, Artificial sequence
  Primer
gatgtcgaatagccgggctgctac

SEQ ID NO: 3 - DNA, 24 bp, Artificial sequence
  Primer
aagaacatcgattttccatggcag

SEQ ID NO: 4 - DNA, 23 bp, Artificial sequence
  Primer
cgactcactatagggagagcggc

SEQ ID NO: 5 - DNA, 40 bp, Artificial sequence
  Primer
ctcgagtttttcagcaagattgccaagtcgttcaacacag

SEQ ID NO: 6 - DNA, 40 bp, Artificial sequence
  Primer
aggagatcttctagaaagatctgagacagcagcttgtcac

SEQ ID NO: 7 - DNA, 42 bp, Artificial sequence
  Primer
gtgacaagctgctgtctcagatctttctagaagatctcctac

SEQ ID NO: 8 - DNA, 45 bp, Artificial sequence
  Primer
ctgtgttgaacgacttggcaatcttgctgaaaaactcgagccatc

SEQ ID NO: 9 - DNA, 20 bp, Artificial sequence
  Primer
tgccaagtcgttcaacacag

SEQ ID NO: 10 - DNA, 20 bp, Artificial sequence
  Primer
ctgagacagcagcttgtcac

SEQ ID NO: 11 - DNA, 28 bp, Artificial sequence
  Primer
gccttggcattcaacatctcagggaatc

SEQ ID NO: 12 - DNA, 20 bp, Artificial sequence
  Primer
gcacgcccttagcttcagag

SEQ ID NO: 13 - DNA, 40 bp, Artificial sequence
  Primer
gactgagatgacggaagaggcccgaacataccagtttcgc

SEQ ID NO: 14 - DNA, 40 bp, Artificial sequence
  Primer
aagcttagtgagatccgcgtgaacaaacgacccaacaccc

SEQ ID NO: 15 - DNA, 41 bp, Artificial sequence
  Primer
gggtgttgggtcgtttgttcacgcggatctcactaagcttc

SEQ ID NO: 16 - DNA, 40 bp, Artificial sequence
  Primer
gcgaaactggtatgttcgggcctcttccgtcatctcagtc

SEQ ID NO: 17 - DNA, 20 bp, Artificial sequence
  Primer
gccagtgcaacaagtatgag

SEQ ID NO: 18 - DNA, 20 bp, Artificial sequence
  Primer
gaccaggctaaacgcatcac

SEQ ID NO: 19 - DNA, 18 bp, Artificial sequence
  Primer
gagagtgggacctgattc

SEQ ID NO: 20 - DNA, 22 bp, Artificial sequence
  Primer
ctgctctcaacaccgagtgtag

SEQ ID NO: 21 - DNA, 58 bp, Artificial sequence
  Primer
attgtcctccaagtaactcgcggtggcagtatagtgagtcgtattacaattcactggc

SEQ ID NO: 22 - DNA, 57 bp, Artificial sequence
  Primer
cggccagtgaattgtaatacgactcactatactgccaccgcgagttacttggaggac

SEQ ID NO: 23 - DNA, 60 bp, Artificial sequence
  Primer
cgaacactgccatcatggttcaacctcacgaggccgtattgaacagtttggacactcttg

SEQ ID NO: 24 - DNA, 60 bp, Artificial sequence
  Primer
caagagtgtccaaactgttcaatacggcctcgtgaggttgaaccatgatggcagtgttcg

SEQ ID NO: 25 - DNA, 58 bp, Artificial sequence
  Primer
actcatgatatgtatgctcgaagcacagaaccgggtatactagtgatttgaacaaacg

SEQ ID NO: 26 - DNA, 60 bp, Artificial sequence
  Primer
ggtcgtttgttcaaatcactagtatacccggttctgtgcttcgagcatacatatcatgag

SEQ ID NO: 27 - DNA, 57 bp, Artificial sequence
  Primer
ctcccatatggtcgacctgcaggcggccagtgcgccgatctcacgccgtgctcatgc

SEQ ID NO: 28 - DNA, 60 bp, Artificial sequence
  Primer
agcatgagcacggcgtgagatcggcgcactggccgcctgcaggtcgaccatatgggagag

SEQ ID NO: 29 - DNA, 30 bp, Artificial sequence
  Primer
ctgccaccgcgagttacttggaggacaata

SEQ ID NO: 30 - DNA, 31 bp, Artificial sequence
  Primer
tgcgccgatctcacgccgtgctcatgcttag

SEQ ID NO: 31 - DNA, 32 bp, Artificial sequence
  Primer
ggcatcatcatgaccacaagaaggaggagaac

SEQ ID NO: 32 - DNA, 30 bp, Artificial sequence
  Primer
gaaagtcgggcgagtccgtttcttctcagc

SEQ ID NO: 33 - DNA, 49 bp, Artificial sequence
  Primer
tcagaagcgtatttcatcagttatcggccgcctgcaggtcgaccatatg

SEQ ID NO: 34 - DNA, 49 bp, Artificial sequence
  Primer
atctatgctcagacagggctgacgtattatgaaatcccgcggccatggc

SEQ ID NO: 35 - DNA, 52 bp, Artificial sequence
  Primer
cggccgccatggccgcgggatttcataatacgtcagccctgtctgagcatag

SEQ ID NO: 36 - DNA, 57 bp, Artificial sequence
  Primer
cagtgtacttcattgatcagaattcgaacactagacaatgtttcaaaggttcttaag

SEQ ID NO: 37 - DNA, 49 bp, Artificial sequence
  Primer
gccttaagaacctttgaaacattgtctagtgttcgaattctgatcaatg

SEQ ID NO: 38 - DNA, 51 bp, Artificial sequence
  Primer
ctcatggcgtggcttatgcgtaaaataaccggaaacaaacgacccaacacc

SEQ ID NO: 39 - DNA, 46 bp, Artificial sequence
  Primer
cgcacgggtgttgggtcgtttgtttccggttattttacgcataagc

SEQ ID NO: 40 - DNA, 49 bp, Artificial sequence
  Primer
ctctcccatatggtcgacctgcaggcggccgataactgatgaaatacgc

SEQ ID NO: 41 - DNA, 21 bp, Artificial sequence
  Primer
tcataatacgtcagccctgtc

SEQ ID NO: 42 - DNA, 23 bp, Artificial sequence
  Primer
ataactgatgaaatacgcttctg

SEQ ID NO: 43 - DNA, 28 bp, Artificial sequence
  Primer
cagcctcttctagtccgctcacaattcc

SEQ ID NO: 44 - DNA, 18 bp, Artificial sequence
  Primer
gcgttcggtttcagaatc

SEQ ID NO: 45 - DNA, 50 bp, Artificial sequence
  Primer
ttcttatcggaggttttatatacgtggccgcctgcaggtcgaccatatgg

SEQ ID NO: 46 - DNA, 51 bp, Artificial sequence
  Primer
atagtgctttccctagcttgaaatcccgcggccatggcggccgggagcatg

SEQ ID NO: 47 - DNA, 42 bp, Artificial sequence
  Primer
gccgccatggccgcgggatttcaagctagggaaagcactatc

SEQ ID NO: 48 - DNA, 56 bp, Artificial sequence
  Primer
cttcattgatcagaattcgaacactagacaatgttggagaggttaggcactattgg

SEQ ID NO: 49 - DNA, 50 bp, Artificial sequence
  Primer
gtgcctaacctctccaacattgtctagtgttcgaattctgatcaatgaag

SEQ ID NO: 50 - DNA, 48 bp, Artificial sequence
  Primer
atggcgtggtatatgcttgaaataactggaaacaaacgacccaacacc

SEQ ID NO: 51 - DNA, 59 bp, Artificial sequence
  Primer
aacgcacgggtgttgggtcgtttgtttccagttatttcaagcatataccacgccatgag

SEQ ID NO: 52 - DNA, 47 bp, Artificial sequence
  Primer
tctggaggatcatgtgtgttgtcggccgcctgcaggtcgaccatatg

SEQ ID NO: 53 - DNA, 22 bp, Artificial sequence
  Primer
tcaagctagggaaagcactatc

SEQ ID NO: 54 - DNA, 23 bp, Artificial sequence
  Primer
acgtatataaaacctccgataag

SEQ ID NO: 55 - DNA, 18 bp, Artificial sequence
  Primer
agcggtgtacaggttagg

SEQ ID NO: 56 - DNA, 47 bp, Artificial sequence; identical to SEQ ID NO: 52
  Primer

SEQ ID NO: 57 - DNA, 53 bp, Artificial sequence
  Primer
aaagtgccgagcaccccttacagaatcccgcggccatggcggccgggagcatg

SEQ ID NO: 58 - DNA, 44 bp, Artificial sequence
  Primer
gccgccatggccgcgggattctgtaaggggtgctcggcactttc

SEQ ID NO: 59 - DNA, 47 bp, Artificial sequence
  Primer
cattgatcagaattcgaacactgggctgtgttgaacagtttagacac

SEQ ID NO: 60 - DNA, 45 bp, Artificial sequence
  Primer
gagtgtctaaactgttcaacacagcccagtgttcgaattctgatc

SEQ ID NO: 61 - DNA, 55 bp, Artificial sequence
  Primer
gacatatgtccaaagctacagggaacaaacgacccaacacccgtgcgttttattc

SEQ ID NO: 62 - DNA, 44 bp, Artificial sequence
  Primer
acgggtgttgggtcgtttgttccctgtagctttggacatatgtc

SEQ ID NO: 63 - DNA, 49 bp, Artificial sequence
  Primer
ccatatggtcgacctgcaggcggccgacaacacacatgatcctccagag

SEQ ID NO: 64 - DNA, 24 bp, Artificial sequence
  Primer
ctgtaaggggtgctcggcactttc

SEQ ID NO: 65 - DNA, 27 bp, Artificial sequence
  Primer
aaaccaacagttcctcgacatattttg

SEQ ID NO: 66 - DNA, 18 bp, Artificial sequence
  Primer
ctcatgttgtgggttctg

SEQ ID NO: 67 - DNA, 42 bp, Artificial sequence
  Primer
gcaacgatgatatgattgacgggaatcccgcggccatggcgg

SEQ ID NO: 68 - DNA, 43 bp, Artificial sequence
  Primer
cagacgtcttgttcgcttcggccgcctgcaggtcgaccatatg

SEQ ID NO: 69 - DNA, 42 bp, Artificial sequence
  Primer
ccgccatggccgcgggattcccgtcaatcatatcatcgttgc

SEQ ID NO: 70 - DNA, 47 bp, Artificial sequence
  Primer
gccatcatggttcaacctcacgtgaatatagattcgaatgaaaggag

SEQ ID NO: 71 - DNA, 47 bp, Artificial sequence
  Primer
ctcctttcattcgaatctatattcacgtgaggttgaaccatgatggc

SEQ ID NO: 72 - DNA, 41 bp, Artificial sequence
  Primer
gggttggaaggcgaaagagacccgggtatactagtgatttg

SEQ ID NO: 73 - DNA, 41 bp, Artificial sequence
  Primer
caaatcactagtatacccgggtctctttcgccttccaaccc

SEQ ID NO: 74 - DNA, 43 bp, Artificial sequence
  Primer
catatggtcgacctgcaggcggccgaagcgaacaagacgtctg

SEQ ID NO: 75 - DNA, 24 bp, Artificial sequence
  Primer
cccgtcaatcatatcatcgttgcc

SEQ ID NO: 76 - DNA, 19 bp, Artificial sequence
  Primer
gaagcgaacaagacgtctg

SEQ ID NO: 77 - DNA, 22 bp, Artificial sequence
  Primer
caagatctcctcgacacaatgg

SEQ ID NO: 78 - DNA, 55 bp, Artificial sequence
  Primer
cagtttgtaaactcggctccgaacaggccgcctgcaggtcgaccatatgggagag

SEQ ID NO: 79 - DNA, 50 bp, Artificial sequence
  Primer
ctgttgcccttcgagctggaaatcccgcggccatggcggccgggagcatg

SEQ ID NO: 80 - DNA, 48 bp, Artificial sequence
  Primer
ccggccgccatggccgcgggatttccagctcgaagggcaacagggtag

SEQ ID NO: 81 - DNA, 49 bp, Artificial sequence
  Primer
ttgatcagaattcgaacactacttcatcctttcccaacccatgaaatcc

SEQ ID NO: 82 - DNA, 45 bp, Artificial sequence
  Primer
gggttgggaaaggatgaagtagtgttcgaattctgatcaatgaag

SEQ ID NO: 83 - DNA, 57 bp, Artificial sequence
  Primer
tcttcccattagatcttatttcacaaacaaacgacccaacacccgtgcgttttattc

SEQ ID NO: 84 - DNA, 51 bp, Artificial sequence
  Primer
acgcacgggtgttgggtcgtttgtttgtgaaataagatctaatgggaagag

SEQ ID NO: 85 - DNA, 47 bp, Artificial sequence
  Primer
tatggtcgacctgcaggcggcctgttcggagccgagtttacaaactg

SEQ ID NO: 86 - DNA, 20 bp, Artificial sequence
  Primer
tccagctcgaagggcaacag

SEQ ID NO: 87 - DNA, 19 bp, Artificial sequence
  Primer
tgttcggagccgagtttac

SEQ ID NO: 88 - DNA, 25 bp, Artificial sequence
  Primer
gtcttggttagaacgtgagcagata

SEQ ID NO: 89 - DNA, 52 bp, Artificial seequence
  Primer
ggctgcaatgtctcttctcgatacttacggccgcctgcaggtcgaccatatg

SEQ ID NO: 90 - DNA, 50 bp, Artificial sequence
  Primer
tggtgtccgcgggcacaatttcttaatcccgcggccatggcggccgggag

SEQ ID NO: 91 - DNA, 50 bp, Artificial sequence
  Primer
catgctcccggccgccatggccgcgggattaagaaattgtgcccgcggac

SEQ ID NO: 92 - DNA, 59 bp, Artificial sequence
  Primer
gtgtacttcattgatcagaattcgaacactacttcatcccttcccaacccatgaaatcc

SEQ ID NO: 93 - DNA, 51 bp, Artificial sequence
  Primer
ggatttcatgggttgggaagggatgaagtagtgttcgaattctgatcaatg

SEQ ID NO: 94 - DNA, 47 bp, Artificial sequence
  Primer
gtggctcttcccattagatcgtatttcacgaacaaacgacccaacac

SEQ ID NO: 95 - DNA, 51 bp, Artificial sequence
  Primer
acgcacgggtgttgggtcgtttgttcgtgaaatacgatctaatgggaagag

SEQ ID NO: 96 - DNA, 48 bp, Artificial sequence
  Primer
ccatatggtcgacctgcaggcggccgtaagtatcgagaagagacattg

SEQ ID NO: 97 - DNA, 20 bp, Artificial sequence
  Primer
aagaaattgtgcccgcggac

SEQ ID NO: 98 - DNA, 23 bp, Artificial sequence
  Primer
gtaagtatcgagaagagacattg

SEQ ID NO: 99 - DNA, 19 bp, Artificial sequence
  Primer
agctgtagtatttgacttc

SEQ ID NO: 100 - DNA, 1560 bp, Starmerella bombicola
atgaacattaatttctctgacgtgctcgtgctaggaggcatcagcgtgagctttttgctcgcctaccaggcgatttacttttatttcatttactcgccacgagccaaaaagctcggttgcgctcttccaccggtcttcttctctttcccactcggaataccggaggtcatacgtcttgtgaacgcctggttcaacgatgatctccttgagtatttcaccttcaaattcgaggagttccagcgcaaaaccggattccaatcagtcgctgggcaactatggattgggactattgagcccgagaacatcaagactatgctcgctacttcatttaaagactactccctaggcttccgttacgaggccatgtacggccttctcggaaatggcattttcactctcagtggtgagggctggaagcacagccgcgctttgttgcgtccgcaatttagtcgtgagcaagtctctcaccttgaatcaatgcgcacacacatcaatatgttgatcaacaaccacttcaagggtggcaaagtcgtcgatgctcaggttttgttccacaatctaaccattgatactgctaccgaattcctattcggagagagcaccaacactcttgaccctgctcttgctcagcatggattccctggacctaagggtcttgtaaccggtgagcagtttgctgaggcttttacctctgctctcgaattgctttctgtgcgagttatggccggcgccgcatggttcctcgtttggacccccaaattctggcgctcatgcaaagtctgccacaacttcattgattacttcgttttcaaggctctggccactcctatggagaaggaccaggaagctgatcgctacgtctttattcgagaactcacaaaggagacctctgacccacgggtcatccgcgaccaggccctcaacatcctcttggctggtcgtgataccactgcggcacttctcagcttcaccacctactaccttggtgcctaccctgaggtctacgatgagcttcgcgaggctgttattgcggacttcggcaaggaagatgctgagccccctacgtttgagcagcttaagcagtgcaaggtgctacagaacgtcattcgggaagttttgcgattgcacccgaatgtgcccctcaacttccgcgaggccattaccgatactaagttccccacaggaggcggcccgaatggagaccagcccgttttcgttcccaagggacagaaagtgttttacgccacctacgtcatgcagcgaaatgagggtctctggggtcctgactccacaacattccgccctgaccgctggaacgagtcaagagaggccatcgcatccggatgggactacattcctttcaacggcggccctcgtatttgcctgggtcagcagttcgctctcacagaggcgagctacacgctcgtgcgtatctgccaagagttctccaggattgaggttctccaccctgatgttattacctccaggaacgtgatgaaacagcgcatgcgtttgaccaactcttccagcggcggcgtcatagcgaagttcattcgctag

SEQ ID NO: 101 - protein, 519 aa, Starmerella bombicola
MNINFSDVLVLGGISVSFLLAYQAIYFYFIYSPRAKKLGCALPPVFFSFPLGIPEVIRLVNAWFNDDLLEYFTFKFEEFQRKTGFQSVAGQLWIGTIEPENIKTMLATSFKDYSLGFRYEAMYGLLGNGIFTLSGEGWKHSRALLRPQFSREQVSHLESMRTHINMLINNHFKGGKVVDAQVLFHNLTIDTATEFLFGESTNTLDPALAQHGFPGPKGLVTGEQFAEAFTSALELLSVRVMAGAAWFLVWTPKFWRSCKVCHNFIDYFVFKALATPMEKDQEADRYVFIRELTKETSDPRVIRDQALNILLAGRDTTAALLSFTTYYLGAYPEVYDELREAVIADFGKEDAEPPTFEQLKQCKVLQNVIREVLRLHPNVPLNFREAITDTKFPTGGGPNGDQPVFVPKGQKVFYATYVMQRNEGLWGPDSTTFRPDRWNESREAIASGWDYIPFNGGPRICLGQQFALTEASYTLVRICQEFSRIEVLHPDVITSRNVMKQRMRLTNSSSGGVIAKFIR

SEQ ID NO: 102 - DNA, 1560 bp, Starmerella bombicola
atgtttattggactctcagacgcccttgcgctgggcggcatcgctctatgctttttgctcgcctaccaggtaatctatttctacttcgtttactcgccgcgggctagaaaacttggttgcgctcctccccaaatcgatttctctatcccgtttgggttgccttcactttacatgtttgcgaaagctttatgggaggatgatgttttggagtacgcctgccgcaggtttgcaggcatgaaggtcagaactgggcttcaaactgtcgctggccagctatggatagcaactatcgagccggagaacatcaagaccgtacttgccacctcgttcaatgactactcccttggcttccgttataatgccctatacggccttctcggaaatggtattttcacccttagtggtgatggctggaagcacagtcgtgctttgttgcgtccgcagttcagtcgtgagcaagtttctcacttggactccatgcgtacacacatcaacttgatgatcaacaaccatttcaaaggcggccacgtcgtagacgcacaggctcgataccacaatttgaccatcgatactgcgactgaattccttttcggtgagagcactaacacactcgaccctgttcttgcacagcaaggactccctggtcctaagggcaccgttaccggagagcagtttgctgaagctttcacctccgctcttcaagtgctgagtgtccgagttatggccggctccgcatggttcctcatttggactcctaaattctggcgctcgtgcaaggtgtgccacaacttcattgactacttcgtttacaaggccttggccactccgatggagaagggccaagaggctgatcgctatgtttttattcgagagctcacaaaggagacttctgacccaagagtcatccgtgaccaggctctaaacatcctgctggctggtcgtgataccactgcggcactcctcagttttaccacctattaccttggcgcataccctgaagtctacgatgagctccgtgaggcagtcattgcggactttggctctgaggacgctgagccccctacctttgagcagctcaagcagtgcaaggtgctgcagaatgtcattcgcgaggttttacgtttgcaccctaatgtgccgctcaacttccgccaggctattactgatactaagctccccactggtggtggcccgaatagagaccagcccgtctttgttccaaagggacagaaagtgttctacgccacctacgtcatgcagcgagatccggaaatatggggccccgactctacaagcttccgccctgatcgatggaatgagccgagagaggctcttgcatcaggttgggattatattcctttcaatggcggccctcgcatttgtatcggtcagcagttcgctctcactgaggctagctacacacttgtccgtatctgtcaggagttctccaggattgaggttcttcatcccgatgtcatcacttctaggaactcaatgaagcagcgcatgcgcttaaccaattctgccagcggcggcgtaatggcaaagttcatccgctag

SEQ ID NO: 103 - protein, 519 aa, Starmerella bombicola
MFIGLSDALALGGIALCFLLAYQVIYFYFVYSPRARKLGCAPPQIDFSIPFGLPSLYMFAKALWEDDVLEYACRRFAGMKVRTGLQTVAGQLWIATIEPENIKTVLATSFNDYSLGFRYNALYGLLGNGIFTLSGDGWKHSRALLRPQFSREQVSHLDSMRTHINLMINNHFKGGHVVDAQARYHNLTIDTATEFLFGESTNTLDPVLAQQGLPGPKGTVTGEQFAEAFTSALQVLSVRVMAGSAWFLIWTPKFWRSCKVCHNFIDYFVYKALATPMEKGQEADRYVFIRELTKETSDPRVIRDQALNILLAGRDTTAALLSFTTYYLGAYPEVYDELREAVIADFGSEDAEPPTFEQLKQCKVLQNVIREVLRLHPNVPLNFRQAITDTKLPTGGGPNRDQPVFVPKGQKVFYATYVMQRDPEIWGPDSTSFRPDRWNEPREALASGWDYIPFNGGPRICIGQQFALTEASYTLVRICQEFSRIEVLHPDVITSRNSMKQRMRLTNSASGGVMAKFIR

SEQ ID NO: 104 - DNA, 1560 bp, Starmerella bombicola
atgtttattggactctcagacgcccttgcgctgggcggcatcgctctatgctttttgctcgcctaccaagtaatctatttctacttcgtttactcgccgcgggctagaaaacttggttgcgctcctccccaaatcgatttctctatcccgtttgggttgccttcactttacatgtttgcgaaagctttatgggaggatgatgttttggagtacgcctgccgcaggtttgcaggcatgaaggtcagaactgggcttcaaactgtcgctggccagctatggatagcaactatcgagccggagaacatcaagaccgtacttgccacctcgttcaatgactactcccttggcttccgttataatgccctatacggccttctcggaaatggtattttcacccttagtggtgatggctggaagcacagtcgtgctttgttgcgtccgcagttcagtcgtgagcaagtttctcacttggactccatgcgtacacacatcaacttgatgatcaacaaccatttcaaaggcggccacgtcgttgacgcacaggctcgataccacaatttgaccatcgatactgcgactgaattccttttcggtgagagcactaacacactcgaccctgttcttgcacagcaaggactccctggtcctaagggcaccgttaccggagagcagtttgctgaagctttcacctccgctcttcaagtgctgagtgtccgagttatggccggctccgcatggttcctcatttggactcctaaattctggcgctcgtgcaaggtgtgccacaacttcattgactacttcgtatacaaggccttggccactccgatggagaagggccaagaggctgatcgctatgtttttattcgagagctcacaaaggagacttctgacccaagagtcatccgtgaccaggctctaaatatcctgctggctggtcgtgataccactgcggcactcctcagttttaccacctattaccttggcgcatatcctgaggtctacgatgagctccgtgaggcagtcattgcggactttggctctgaggacgctgagccccctacctttgagcagctcaagcagtgcaaggtactgcagaatgtcattcgcgaggttttacgtttgcaccctaatgtgccgctcaacttccgccaggctataactgatactaagctccccactggtggtggcccgaacagagaccagcctgtctttgttccaaagggacagaaagtgttctacgccacctacgtcatgcagcgagatccggaaatatggggccccgactctacaagcttccgccctgatcgatggaatgagccgagagaggctcttgcatcaggttgggattatattcctttcaatggcggccctcgcatttgtatcggtcagcagttcgctctcactgaggctagttacacacttgtccgtatttgtcaggagttctccaggatcgaggttcttcatcccgatgtcatcacttctaggaactcgatgaagcagcgcatgcgcttgaccaattctgctagtggcggcgtaatggcaaggttcatccgctag

SEQ ID NO: 105 - protein, 519 aa, Starmerella bombicola
MFIGLSDALALGGIALCFLLAYQVIYFYFVYSPRARKLGCAPPQIDFSIPFGLPSLYMFAKALWEDDVLEYACRRFAGMKVRTGLQTVAGQLWIATIEPENIKTVLATSFNDYSLGFRYNALYGLLGNGIFTLSGDGWKHSRALLRPQFSREQVSHLDSMRTHINLMINNHFKGGHVVDAQARYHNLTIDTATEFLFGESTNTLDPVLAQQGLPGPKGTVTGEQFAEAFTSALQVLSVRVMAGSAWFLIWTPKFWRSCKVCHNFIDYFVYKALATPMEKGQEADRYVFIRELTKETSDPRVIRDQALNILLAGRDTTAALLSFTTYYLGAYPEVYDELREAVIADFGSEDAEPPTFEQLKQCKVLQNVIREVLRLHPNVPLNFRQAITDTKLPTGGGPNRDQPVFVPKGQKVFYATYVMQRDPEIWGPDSTSFRPDRWNEPREALASGWDYIPFNGGPRICIGQQFALTEASYTLVRICQEFSRIEVLHPDVITSRNSMKQRMRLTNSASGGVMARFIR

SEQ ID NO: 106 - DNA, 1560 bp, Starmerella bombicola
atgattattgatctttcagacgcgctgataataggaggcatcgccctgtgcttcttgctctcctaccaggcgatctacttttactttatttactcgccacgggccaagaagcttggatgcgctcctcctctcattgtgcacgctttcccactgggtttgccgacaattttcggacttataagagcttggcgcaacgacgatcttctccagtacttgagcgacaacttcgctagaatcaggaccagaaccggaatgcaagtaatggccggtcagctgtggctcaacaccattgagccagaaaacatcaaggccatgcttgccacttcgttcaaggatttctcgcttgggttccgctatgaagtcatgcatggcctcctcggagatggtatcttcactctcagtggtgagggctggaaacacagccgtgccttgctacgtccacagttcagccgtgagcaagtctctcacttggactcaatgcgcacacacatcaatttgatgatcaacaaccacttcaaaggtggccaggtcgtcgacgctcaggttctataccataacctgacaatcgacactgccactgaattcctgttcggtgagagcaccaacactcttgaccctgttcttgcacagcagggactaccgggtcctaggggcgttgttactggtgagcagttcgctaacgctttcacctacgctcaagagttgctcagtattcgagtcatggccggctcagcatggttcctcgtctggactcctaagttcaggcgctcgtgcaaggtgtgccacaactttattgactacttcgtctttaaggctctggccactcctatggagaaagaccaggaggctgatcgctatgtattcatccgagaactcactaaggagacttctgacccaaaggttatacgtgaccaggctctcaacatccttttagctggccgcgataccactgcagcactcctcagcttcaccacttactaccttggcgcatatcctgaggtctacgacgagcttcgcgaggcagttcttgcagacttcggccctgccgattctgagccccctacctttgagaggctcaagcagtgcaaggtgttgcagaatgtcatccgcgaggttctgcgattgcacccgaatgtgcccctcaacttccgccaggccatcgttgatactaagttccctactggtggtggcccgaatagagaccagcccatctttgttccaaaaggacagaaggtgttctactccacgtacgtcatgcagcgaagcaaggacatctggggcgctgactccacatcgttccgaccagaacgctggaacgagcccagagaagctcttgcatcaggttgggattacattcctttcaatggtggccctcgcatttgtatcggtcagcagttcgctctcactgaggctagctacacgcttgtccgtatttgccaggagtttaccagaattgaggttcttcatcccgatgtcattacttctaggaaagagatgaagcagcgcatgcgcttgaccaactcggctagcggtggcgtgatggcgagattcattcgttag

SEQ ID NO: 107 - protein, 519 aa, Starmerella bombicola
MIIDLSDALIIGGIALCFLLSYQAIYFYFIYSPRAKKLGCAPPLIVHAFPLGLPTIFGLIRAWRNDDLLQYLSDNFARIRTRTGMQVMAGQLWLNTIEPENIKAMLATSFKDFSLGFRYEVMHGLLGDGIFTLSGEGWKHSRALLRPQFSREQVSHLDSMRTHINLMINNHFKGGQVVDAQVLYHNLTIDTATEFLFGESTNTLDPVLAQQGLPGPRGVVTGEQFANAFTYAQELLSIRVMAGSAWFLVWTPKFRRSCKVCHNFIDYFVFKALATPMEKDQEADRYVFIRELTKETSDPKVIRDQALNILLAGRDTTAALLSFTTYYLGAYPEVYDELREAVLADFGPADSEPPTFERLKQCKVLQNVIREVLRLHPNVPLNFRQAIVDTKFPTGGGPNRDQPIFVPKGQKVFYSTYVMQRSKDIWGADSTSFRPERWNEPREALASGWDYIPFNGGPRICIGQQFALTEASYTLVRICQEFTRIEVLHPDVITSRKEMKQRMRLTNSASGGVMARFIR

SEQ ID NO: 108 - DNA, 1572 bp, Starmerella bombicola
atgattctttatgctgtgctgggcgcattcgccgccttcttgctttacatggatgtactttaccctttcgtgatttaccctctgagagcgcgatggcacaaatgtggttacatccctagagatttgagctggccattggggattccactcaccctggtagttctctcgaagttgaggaaagatatgctgctgcaattcatggcagcgcaagaccttagtcgcccttacaagacatccttacgtcaatttctgggtaaatgggtaatcgccactagagatcctgagaacatcaaggctgttctatccaccaagttcaatgacttctcgctgaaagaaagagggaataggatgaggcatgtaatcggtgatggaatttttacccaagatggcgcaccatggaagcactcgcgagatatgctcaggcctcagttcaccaaggatcaaatcagccgagtggaattgttgagccaccacatcgacgttttgattcgtgaaatcaggaagtcgggaggtaacgtcgagttgcaacgtttattccacctcatgactatggacaccgccactcactttctattcggcgagtccgttggctcgttggaggtcagtggcgaaagcaagggcattgagatcaccgacccaaagactggagagattgtgaacaccgttgattttgttgagtcttatacttttgcaaacaagtttgctctcaagaagattatcctcaacgacttggagtttttagccgacttgacggagccctcgtataagtggcatctgcgccgtgtccacacagtcatggatcactacgttcagctggctttgaaggctactgagaagtatgatcctgatgatgatagcgagaagggagaatactactttagccatgagctggcgaaactcacgagagaccccttgtcgttgagagatcagcttttcaatattctcattgctggccgcgacactaccgcagcaactttgtcctatgccttccactatctaacgaagaatcccgctatctacgccaaggtccgcgaagatgtgctcacggtcttccctaatggagacgcatcattggcgacttacgaggacttgcgaaaggctaagtatctccaaatggtgatcaaggaggtattgcgtcttgcgcctgcggttcccttgaacacgcgtgccgcggttcgtgacacatatctgccacggggcggaggcccagccggaaacctgcccgtttttgttcccaagggcactgctgtcaactaccctacatatattttgcaccgcgatccagatatctatggtgccgacgcgtacgagttcaaccccgagagatggaggcctgagaataagcttccgaatagcccaatgtactcttggggatacattcccttcaatggtggccctcgcatctgcattggacagcagttcgccttgactgagatcgctttgacgatgatcaagctggttctggaatttgagaggctggagcctgccgacgactttgagcccaatcttcaagacaagtcctctttaactgtcatggtcggagggtcgggcgtccgagtgaaactgagttaa

SEQ ID NO: 109 - protein, 523 aa, Starmerella bombicola
MILYAVLGAFAAFLLYMDVLYPFVIYPLRARWHKCGYIPRDLSWPLGIPLTLVVLSKLRKDMLLQFMAAQDLSRPYKTSLRQFLGKWVIATRDPENIKAVLSTKFNDFSLKERGNRMRHVIGDGIFTQDGAPWKHSRDMLRPQFTKDQISRVELLSHHIDVLIREIRKSGGNVELQRLFHLMTMDTATHFLFGESVGSLEVSGESKGIEITDPKTGEIVNTVDFVESYTFANKFALKKIILNDLEFLADLTEPSYKWHLRRVHTVMDHYVQLALKATEKYDPDDDSEKGEYYFSHELAKLTRDPLSLRDQLFNILIAGRDTTAATLSYAFHYLTKNPAIYAKVREDVLTVFPNGDASLATYEDLRKAKYLQMVIKEVLRLAPAVPLNTRAAVRDTYLPRGGGPAGNLPVFVPKGTAVNYPTYILHRDPDIYGADAYEFNPERWRPENKLPNSPMYSWGYIPFNGGPRICIGQQFALTEIALTMIKLVLEFERLEPADDFEPNLQDKSSLTVMVGGSGVRVKLS

SEQ ID NO: 110 - DNA, 1572 bp, Starmerella bombicola
atgattttttatgctgtgcttggcgctgtggtcaccttcttactttacgtagatgtgatctaccctttcgtgatatatcctttaaaagcacgatggcacaaatgtggctccgtacctcgagagcttagctggccattggggattccaaccaccataggagttttttcgaacataaagaaggatctacatcttcaagtcctggcagcgtacgacctcagccggtcttataagacaagcttgcgtcaaagtctcggcacatgggtagttgctacgcgggatcctgagaacatcaaggccgttttgtctaccaagttcaatgacttttcactgaaagagagaggaattcggttaaggcatgtaattggtgatggtatctttacccaagatggtgcaccgtggaagcactcgcgagatatgctcagacctcaattcagtagggaacaaatcagccgcgtggaggtgttgagtcaccacatcgatgttttgattcgtgagatcaaaaagtcgggaggtaatgttgagttgcaacgactattccacctcatgactatggacaccgccacacagtttcttttcggcgaatcaattggctcgctagaagtcagtggcgacagcaagggcattgagattactgacccaaatactggagatattgtgagtaccgttgacttcgttgagtcttatactttcacaaacagatttgctatgaagaaggtattcctgaacaaatgggaattcttggcaaacttgtcgaacccctcatatgagaggcatatgcggcgtgtccacacagtcctggatcactacgttcagctggctttgaaggctactgagaagtatgatcctgaagatgacagcgagaaaggagaatactactttagccatgagctggctaaactcacgagagaccccttgtcgttgcgcaatcagctttttaatatcctgattgctggccgcgacactaccgcagcaacattgtcctatgccttccattacttaacgaagaacccagccatctacgccaaggttcgcgaagatgtgctcaccgtcttccccgatggagacgcctcattggcgacctttgaggacttgcgaaaggccaagtatctccaaatggtaatcaaggaggtattgcgccttgcgcctgcggttcccacaaattcgcgtactgcggttcgtgacacctatctgccacggggtggaggcccagctggaaacctacccgttttcgttcccaagggcactattatcaggtatcctgcatatatcttgcaccgcgatcctgatatatatggtgccgactcgtatgacttcaaccctgagaggtggagacccgagaataagctcccaggtagcccaatgtactcatggggctatattccctttaatggcggccctcgcatttgcgttggacagcagtttgccttgactgaaatcgctttgacaatgatcaagctggttttggaatttgagaggctggagcctgctgatgactttgagcccaatcttcgagataggacctcattaacttccatggtcggagggtcgggcgtccgagtaaaactgagttaa

SEQ ID NO: 111 - protein, 523 aa, Starmerella bombicola
MIFYAVLGAVVTFLLYVDVIYPFVIYPLKARWHKCGSVPRELSWPLGIPTTIGVFSNIKKDLHLQVLAAYDLSRSYKTSLRQSLGTWVVATRDPENIKAVLSTKFNDFSLKERGIRLRHVIGDGIFTQDGAPWKHSRDMLRPQFSREQISRVEVLSHHIDVLIREIKKSGGNVELQRLFHLMTMDTATQFLFGESIGSLEVSGDSKGIEITDPNTGDIVSTVDFVESYTFTNRFAMKKVFLNKWEFLANLSNPSYERHMRRVHTVLDHYVQLALKATEKYDPEDDSEKGEYYFSHELAKLTRDPLSLRNQLFNILIAGRDTTAATLSYAFHYLTKNPAIYAKVREDVLTVFPDGDASLATFEDLRKAKYLQMVIKEVLRLAPAVPTNSRTAVRDTYLPRGGGPAGNLPVFVPKGTIIRYPAYILHRDPDIYGADSYDFNPERWRPENKLPGSPMYSWGYIPFNGGPRICVGQQFALTEIALTMIKLVLEFERLEPADDFEPNLRDRTSLTSMVGGSGVRVKLS

SEQ ID NO: 112 - DNA, 1572 bp, Starmerella bombicola
atgattttttatgctgtgcttggcactgtggtcgccttcttactttacgtagatgtgatctaccctttcgtgatatatcctttaaaggcacgatggcacaaatgtggcttcgtccctcgagagctgagctggccattggggattccagacaccatagcagttttttcgaggataaagaaggatctacatcttcaattcctggcagcgcacgacctcagccggtcttataagacaagcttgcgtcaaactctcggcacatgggtagttgatacgcgagatcctgagaatatcaaggccgttttgtctaccaagttcaatgacttttcactgaaagatagaggaattcggttaaggcaagtaattggtgatggtatttttacccaagatggtgcaccgtggaagcactcgcgagatatgctcagacctcaattcagtagggaacaaattagccgcgtggaggtgttgagtcaccacatcgatgttttgattcgtgagatcaaaaagtcgggaggtaatgttgagttgcaacgactattccacctcatgactatggacactgctacacagtttcttttcggcgaatcaattggctcgctagaagtcagtggcgacagcaagggcattgagattactgacccaaatactggagatattgtgaataccgttgacttcgttgagtcttatacttttgcaaacagatttgctatgaaaaagatattactgaacaaatgggaattcgtggtaaacttgtcgaacccctcatatgagaggcatatgcgacgtgtccacacagtcctggatcactacgttcagctggctttgaaggctactgagaagtatgatcctgaagatgactgcgagaaaggagaatactactttagccatgagctggctaaactcacgagagaccccttgtgcttgcgcaatcagctttttaatatcctgattgctggccgcgacactaccgcagcaacattggcctatgccttccattacttgacgaagaacccagccatctacgccaaggtgcgcgaagatgtgctcaccgtcttccccaatggagatgcctcattggcgacctttgaggacttgcgaaaggccaagtatctccaaatggtaatcaaggaggtattgcgccttgcgcctgtggttcccacaaattcgcgtactgcggttcgtgacacctatctgccacggggtggaggcccagctggaaacctacccgttttcgttcccaagggcacaaatgtcaggtattctgcatatgtcttgcaccgcgatcctgatatatatggtgccgactcgtatgacttcaaccctgagaggtggagacccgagaataagctcccaggtagcccaatgtactcatggggctatattccctttaatggcggccctcgcatttgcgttggacagcagtttgccttgactgaattcgctttgacaatgatcaagctggttttagaatttgagaggctggagcctgctgatgactttgagcccaatcttctagataggacctcattaactgccatggtcggagggtcgggcgtccgagtaaaactgagttaa

SEQ ID NO: 113 - protein, 523 aa, Starmerella bombicola
MIFYAVLGTVVAFLLYVDVIYPFVIYPLKARWHKCGFVPRELSWPLGIPDTIAVFSRIKKDLHLQFLAAHDLSRSYKTSLRQTLGTWVVDTRDPENIKAVLSTKFNDFSLKDRGIRLRQVIGDGIFTQDGAPWKHSRDMLRPQFSREQISRVEVLSHHIDVLIREIKKSGGNVELQRLFHLMTMDTATQFLFGESIGSLEVSGDSKGIEITDPNTGDIVNTVDFVESYTFANRFAMKKILLNKWEFVVNLSNPSYERHMRRVHTVLDHYVQLALKATEKYDPEDDCEKGEYYFSHELAKLTRDPLCLRNQLFNILIAGRDTTAATLAYAFHYLTKNPAIYAKVREDVLTVFPNGDASLATFEDLRKAKYLQMVIKEVLRLAPVVPTNSRTAVRDTYLPRGGGPAGNLPVFVPKGTNVRYSAYVLHRDPDIYGADSYDFNPERWRPENKLPGSPMYSWGYIPFNGGPRICVGQQFALTEFALTMIKLVLEFERLEPADDFEPNLLDRTSLTAMVGGSGVRVKLS